(19) United States
(12) Patent Application Publication
(10) Pub. No.: US 2023/0190893 A1
(43) Pub. Date: Jun. 22, 2023

(54) COMPOSITIONS AND METHODS FOR TREATING AN INHERITED RETINAL DISEASE

(71) Applicant: THE REGENTS OF THE UNIVERSITY OF CALIFORNIA, Oakland, CA (US)

(72) Inventors: SUSIE SUH, Oakland, CA (US); KRZYSZTOF PALCZEWSKI, Oakland, CA (US); ELLIOT H. CHOI, Oakland, CA (US)

(21) Appl. No.: 18/016,428

(22) PCT Filed: Jul. 14, 2021
(86) PCT No.: PCT/US2021/041632

§ 371 Date: Jan. 16, 2023

Related U.S. Application Data
(60) Provisional application No. 63/051,684, filed on Jul. 14, 2020.

(51) Int. Cl. A61K 38/50; A61K 31/7105; C12N 9/78; C12N 15/11; A61K 9/00; A61P 27/02; C12N 9/22; A61K 38/46

(52) U.S. Cl. CPC A61K 38/50; A61K 9/0048; A61K 31/7105; A61K 38/465; A61P 27/02; C12N 9/22; C12N 9/78; C12N 15/11; C12Y 305/04004; C12N 2310/20

(57) ABSTRACT

A method of treating an inherited retinal disease (IRD) associated with a pathogenic point mutation in a mutant allele of an IRD-related gene in the retina or the retinal pigment epithelium (RPE) of a subject in need thereof includes base editing the pathogenic point mutation in the retinal cell or retinal pigment epithelium cell to correct the pathogenic mutation, generate a non-pathogenic point mutation, or modulate expression of an IRD-related gene and restore visual function of subject.

### RELATED APPLICATION

This application claims priority from U.S. Provisional Application No. 63/051,684, filed Jul. 14, 2020, the subject matter of which is incorporated herein by reference in its entirety.

### GOVERNMENT FUNDING

This invention was made with government support under Grant Nos. EY009339, EY027283, EY019312, F30EY029136, T32GM007250, T32EY024236, T32GM007250, and T32GM008803 awarded by The National Institutes of Health. The United States government has certain rights to the invention.

### BACKGROUND

Inherited retinal diseases (IRDs) are a group of binding conditions caused by mutations in more than 250 different genes. Among them, Leber congenital amaurosis (LCA) is a common cause of inherited blindness in childhood with a prevalence of 10%. Most patients with LCA have severe visual impairment throughout infancy or childhood and become legally blind by the third or fourth decade of life due to progressing retinal degeneration. This devastating form of disease had no avenue for treatment until the recent US FDA approval of the first gene augmentation therapy targeted for LCA patients with biallelic mutations in the RPE65 gene. The RPE65 is a critical enzyme in the retinal pigment epithelium (RPE) mediating isomerization of all-trans-retinyl esters into 11-cis-retinol, a key step in the visual cycle. Loss-of-function mutations in the RPE65 gene are one of the common cause of IRDs, making this gene an important target for therapy.

The therapeutic strategy of FDA-approved gene augmentation therapy relies on the subretinal delivery of a functional copy of the RPE65 gene via adeno-associated virus (AAV) to compensate for loss-of-function RPE65 mutations in patients. Although the gene therapy initially improved patients’ visual sensitivity, long-term reports showed a continuation of retinal degeneration and a decrease in visual sensitivity after one year, highlighting a limitation of current gene augmentation therapy. The possible explanation for declining therapeutic effect is attributed to insufficient or declining transgene expression level from the delivered AAV. Moreover, gene augmentation approach is not applicable for targeting other forms of IRDs caused by mutations in large-sized genes that exceed the carrying capacity of viral vectors or gain-of- function mutations.

Genome editing with CRISPR-Cas9 technology has the potential to advance the current gene therapy approach with the ability to correct mutations in the endogenous DNA. In the early stage of CRISPR-Cas9 technology, the ability to correct a point mutation was dependent upon the rate of homology-directed repair (HDR) following the delivery of wild-type (wt) Cas9, corresponding single-guide RNA (sgRNA) and homologous donor sequence.

However, the correction by HDR has shown to be highly ineffective particularly in nondividing cells, and the double-stranded DNA (dsDNA) break formation by Cas9 nuclease generates substantial amounts of undesired indel mutations that abrogates the potential benefit from corrected mutation.

### SUMMARY

This disclosure describes a treatment strategy for an inherited retinal disease (IRD). The strategy relies on a precise correction of a pathogenic point mutation in a mutant allele of an IRD-related gene in the retina or the retinal pigment epithelium (RPE) by subretinal delivery of a base editor (BE) system. The BE system includes a base editor and a guide RNA that targets the pathogenic mutation via viral vector or non-viral vector delivery to generate a point mutation or point mutations in the IRD-related gene. Administration of the base editor and guide RNA can correct the pathogenic mutation, generate a non-pathogenic point mutation, or modulate (e.g., increase) expression of an IRD-related gene.

In some embodiments, the base editing system can be tailored to target a mutant allele of an IRD-related gene that includes a point mutation or single nucleotide polymorphism (SNP) that results in a missense mutation or nonsense mutation. For example, the base editing system was used to target a nonsense mutation in an Rpe65 gene on exon 3 (c.130 C>T; p.R44X) in a mouse model of LCA, also known as an rd12 mouse. The homologous mutation was recently identified as an LCA-causing mutation in humans. Subretinal virus mediated delivery of an adenine base editor (ABE), which can convert adenine to guanine at a targeted region in the co-presence of target-specific single-guide RNA (sgRNA), was found to correct the mutation in the rd12 mouse with an efficiency effective to restore retinal and visual function at near normal levels. The ABE system, in contrast to Cas9-induced homologous recombination, enables a single base conversion without making a dsDNA breaks, thereby minimizing the formation of indel mutations and off-target effects.

In some embodiments, a method of treating an inherited retinal disease (IRD) associated with a pathogenic point mutation in a mutant allele of an IRD-related gene in the retina or the retinal pigment epithelium (RPE) of a subject in need thereof includes base editing the pathogenic point mutation in the retinal cell or retinal pigment epithelium cell to correct the pathogenic mutation, generating a non-pathogenic point mutation, or modulating expression of an IRD-related gene, and restoring visual function of subject.

In some embodiments, the pathogenic mutation is a nonsense or missense mutation and the base editing increases expression of a visual cycle protein whose expression was suppressed by mutation of an IRD-related gene in the cell by at least about 4%, 5%, 6%, 7%, 8%, 9%, 10%, 20%, 30%, 40% or more.

In other embodiments, the method causes less than 3%, less than 2%, or less than 1% indel formation.

In some embodiment, the pathogenic mutation is nonsense or missense mutation of an IRD related gene. The IRD related gene can be ABCA4, AIPL1, CABP4, CEP290, CLUAP1, CRB1, CRX, GDF6, GUCY2D, IFT140, IQCB1, KCNJ13, LCAS, LRAT, NMNAT1, PRPH2, RD3, RDH12, RHO, RPE65, RPGRIP1, SPATA7, and TULP1.

In some embodiments, the IRD can include chorioretinal atrophy or degeneration, cone or cone-rod dystrophy, congenital stationary night blindness, Leber congenital amaurosis, macular degeneration, ocular-retinal developmental disease, optic atrophy, retinitis pigmentosa, syndromic/systemic diseases with retinopathy, sorsby macular dystrophy, age-related macular degeneration, doyne honeycomb macular disease, juvenile macular degeneration, Stargardt disease, or retinitis pigmentosis.

In some embodiments, the base editing can be performed by subretinal injecting at least one vector encoding a base editor and guide RNA that hybridizes to or is complementary to a target nucleic acid sequence that includes the point mutation in the IRD-related gene.

In some embodiment, the pathogenic mutation is a nonsense or missense mutation of an RPE65 gene.

In some embodiments, the base editing can be performed by subretinal injecting at least one vector encoding a base editor and guide RNA that hybridizes to or i complementary to a target nucleic acid sequence of the mutant RPE65, which includes the point mutation.

In some embodiments, the pathogenic mutation comprises a C to T missense or nonsense mutation of a RPE65 gene. Deamination of the A complementary to the T by the base editor and the guide RNA corrects the C to T mutation.

In some embodiments, the nucleic acid sequence of the target sequence can include at least one of:

5′-CTCACTGGCAGTCTCCTCTGATGTGGGCCA-3′
(SEQ ID NO: 1);

5′-CTCACCGGCAGTCTCCTCTGATGTGGGCCA-3′
(SEQ ID NO:2);

5′-TCACTGGCAGTCTCCTCTGATGTGGGCCAG-3′
(SEQ ID NO: 3);

5′-CACTGGCAGTCTCCTCTGATGTGGGCCAGG-3′
(SEQ ID NO: 4);

5′-ACTGGCAGTCTCCTCTGATGTGGGCCAGGG-3′
(SEQ ID NO: 5);

5′-GCTCACTGGCAGTCTCCTCTGATGTGGGCC-3′
(SEQ ID NO: 6);

5′-GGCTCACTGGCAGTCTCCTCTGATGTGGGC-3′
(SEQ ID NO: 7);

5′-TGGCTCACTGGCAGTCTCCTCTGATGTGGG-3′
(SEQ ID NO: 8);

5′-TCACCGGCAGTCTCCTTTGATGTGGGCCAG-3′
(SEQ ID NO: 9);

5′-CACCGGCAGTCTCCTTTGATGTGGGCCAGG-3′
(SEQ ID NO: 10);

5′-ACCGGCAGTCTCCTTTGATGTGGGCCAGGG-3′
(SEQ ID NO: 11);

5′-GCTCACCGGCAGTCTCCTTTGATGTGGGCC-3′
(SEQ ID NO: 12);

5′-GGCTCACCGGCAGTCTCCTTTGATGTGGGC-3′
(SEQ ID NO: 13); or

5′-TGGCTCACCGGCAGTCTCCTTTGATGTGGGC-3′
(SEQ ID NO: 14)

In some embodiments, the nucleic acid sequence of DNA encoding the guide sequence can include at least one of:

5′-ATCAGAGGAGACTGCCAGTG-3′ (SEQ ID NO: 15),

5′-CATCAGAGGAGACTGCCAGT-3′ (SEQ ID NO: 16),

5′-ACATCAGAGGAGACTGCCAG-3′ (SEQ ID NO: 17),

5′-CACATCAGAGGAGACTGCCA-3′ (SEQ ID NO: 18),

5′-CCACATCAGAGGAGACTGCC-3′ (SEQ ID NO: 19),

5′-ATCAAAGGAGACTGCCGGTG-3′ (SEQ ID NO: 20),

5′-CATCAAAGGAGACTGCCGGT-3′ (SEQ ID NO: 21),

5′-ACATCAAAGGAGACTGCCGG-3′ (SEQ ID NO: 22),

5′-CACATCAAAGGAGACTGCCG-3′ (SEQ ID NO: 23), or

5′-CCACATCAAAGGAGACTGCC-3 ′ (SEQ ID NO: 24).

In other embodiments, the nucleic acid sequence of the guide sequence can include at least one of:

5′-AUCAGAGGAGACUGCCAGUG-3′ (SEQ ID NO: 25),

5′-CAUCAGAGGAGACUGCCAGU-3′ (SEQ ID NO: 26),

5′-ACAUCAGAGGAGACUGCCAG-3′ (SEQ ID NO: 27),

5′-CACAUCAGAGGAGACUGCCA-3′ (SEQ ID NO: 28),

5′-CCACAUCAGAGGAGACUGCC-3′ (SEQ ID NO: 29),

5′-AUCAAAGGAGACUGCCGGUG-3′ (SEQ ID NO: 30),

5′-CAUCAAAGGAGACUGCCGGU-3′ (SEQ ID NO: 31),

5′-ACAUCAAAGGAGACUGCCGG-3′ (SEQ ID NO: 32),

5′-CACAUCAAAGGAGACUGCCG-3′ (SEQ ID NO: 33), or

5′-CCACAUCAAAGGAGACUGCC-3′ (SEQ ID NO: 34.

Other embodiments described herein relate to a method of restoring cone function or prolonging cone survival in a subject with an IRD-related cone or cone-rod dystrophy associated with a pathogenic point mutation in a mutant allele of an IRD-related gene in the retina or the retinal pigment epithelium (RPE). The method can include base editing the pathogenic mutated gene of a retinal cell or retinal pigment epithelium (RPE) cell to restore cone function or prolong cone survival of the subject.

In some embodiments, the pathogenic mutation is a nonsense or missense mutation and the base editing increases expression of a visual cycle protein whose expression was suppressed by the missense or nonsense gene mutation in the cell by at least about 4%, 5%, 6%, 7%, 8%, 9%, 10%, 20%, 30%, 40% or more.

In other embodiments, the method causes less than 3%, less than 2%, or less than 1% indel formation.

In some embodiment, the pathogenic mutation is a nonsense or missense mutation of an RPE65 gene.

In some embodiments, the base editing can be performed by subretinal injecting at least one vector encoding a base editor and guide RNA that hybridizes to or is complementary to a target nucleic acid sequence of the mutant RPE65, which includes the point mutation.

In some embodiments, the pathogenic mutation comprises a C to T missense or nonsense mutation of the RPE65 gene. Deamination of the A complementary to the T by the base editor and the guide RNA corrects the C to T mutation.

In some embodiments, the nucleic acid sequence of the target sequence can include at least one of:

5′-CTCACTGGCAGTCTCCTCTGATGTGGGCCA -3′
(SEQ ID NO: 1);

5′-CTCACCGGCAGTCTCCTCTGATGTGGGCCA -3′
(SEQ ID NO:2);

5′-TCACTGGCAGTCTCCTCTGATGTGGGCCAG-3′
(SEQ ID NO: 3);

5′-CACTGGCAGTCTCCTCTGATGTGGGCCAGG-3′
(SEQ ID NO: 4);

5′-ACTGGCAGTCTCCTCTGATGTGGGCCAGGG-3′
(SEQ ID NO: 5);

5′-GCTCACTGGCAGTCTCCTCTGATGTGGGCC-3′
(SEQ ID NO: 6);

5′-GGCTCACTGGCAGTCTCCTCTGATGTGGGC-3′
(SEQ ID NO: 7);

5′-TGGCTCACTGGCAGTCTCCTCTGATGTGGG-3′
(SEQ ID NO: 8);

5′-TCACCGGCAGTCTCCTTTGATGTGGGCCAG-3′
(SEQ ID NO: 9);

5′-CACCGGCAGTCTCCTTTGATGTGGGCCAGG-3′
(SEQ ID NO: 10);

5′-ACCGGCAGTCTCCTTTGATGTGGGCCAGGG-3′
(SEQ ID NO: 11);

5′-GCTCACCGGCAGTCTCCTTTGATGTGGGCC-3′
(SEQ ID NO: 12);

5′-GGCTCACCGGCAGTCTCCTTTGATGTGGGC-3′
(SEQ ID NO: 13); or

5′-TGGCTCACCGGCAGTCTCCTTTGATGTGGGC-3′
(SEQ ID NO: 14)

In some embodiments, the nucleic acid sequence of DNA encoding the guide sequence can include at least one of:

5′-ATCAGAGGAGACTGCCAGTG-3 ′ (SEQ ID NO: 15),

5′-CATCAGAGGAGACTGCCAGT-3 ′ (SEQ ID NO: 16),

5′-ACATCAGAGGAGACTGCCAG-3 ′ (SEQ ID NO: 17),

5′-CACATCAGAGGAGACTGCCA-3 ′ (SEQ ID NO: 18),

5′-CCACATCAGAGGAGACTGCC-3 ′ (SEQ ID NO: 19),

5′-ATCAAAGGAGACTGCCGGTG-3 ′ (SEQ ID NO: 20),

5′-CATCAAAGGAGACTGCCGGT-3 ′ (SEQ ID NO: 21),

5′-ACATCAAAGGAGACTGCCGG-3 ′ (SEQ ID NO: 22),

5′-CACATCAAAGGAGACTGCCG-3 ′ (SEQ ID NO: 23), or

5′-CCACATCAAAGGAGACTGCC-3 ′ (SEQ ID NO: 24).

In other embodiments, the nucleic acid sequence of the guide sequence can include at least one of:

5′-AUCAGAGGAGACUGCCAGUG-3 ′ (SEQ ID NO: 25),

5′-CAUCAGAGGAGACUGCCAGU-3 ′ (SEQ ID NO: 26),

5′-ACAUCAGAGGAGACUGCCAG-3 ′ (SEQ ID NO: 27),

5′-CACAUCAGAGGAGACUGCCA-3 ′ (SEQ ID NO: 28),

5′-CCACAUCAGAGGAGACUGCC-3 ′ (SEQ ID NO: 29),

5′-AUCAAAGGAGACUGCCGGUG-3 ′ (SEQ ID NO: 30),

5′-CAUCAAAGGAGACUGCCGGU-3 ′ (SEQ ID NO: 31),

5′-ACAUCAAAGGAGACUGCCGG-3 ′ (SEQ ID NO: 32),

5′-CACAUCAAAGGAGACUGCCG-3 ′ (SEQ ID NO: 33), or

5′-CCACAUCAAAGGAGACUGCC-3 ′ (SEQ ID NO: 34).

In other embodiments, base editing the pathogenic mutated gene of a retinal cell or retinal pigment epithelium (RPE) cell can increase arrestin expression in the retina cells or retinal pigment epithelium cells of the subject being treated.

Still other embodiments relate to a complex that includes a fusion protein comprising a nucleic acid programmable DNA binding protein and an adenosine deaminase and a guide sequence comprising the nucleic sequence of at least one of:

5′-AUCAGAGGAGACUGCCAGUG-3 ′ (SEQ ID NO: 25),

5′-CAUCAGAGGAGACUGCCAGU-3 ′ (SEQ ID NO: 26),

5′-ACAUCAGAGGAGACUGCCAG-3 ′ (SEQ ID NO: 27),

5′-CACAUCAGAGGAGACUGCCA-3 ′ (SEQ ID NO: 28),

5′-CCACAUCAGAGGAGACUGCC-3 ′ (SEQ ID NO: 29),

5′-AUCAAAGGAGACUGCCGGUG-3 ′ (SEQ ID NO: 30),

5′-CAUCAAAGGAGACUGCCGGU-3 ′ (SEQ ID NO: 31),

5′-ACAUCAAAGGAGACUGCCGG-3 ′ (SEQ ID NO: 32),

5′-CACAUCAAAGGAGACUGCCG-3 ′ (SEQ ID NO: 33), or

5′-CCACAUCAAAGGAGACUGCC-3 ′ (SEQ ID NO: 34).

Other embodiments relate to a guide sequence comprising the nucleic sequence of at least one of:

5′-AUCAGAGGAGACUGCCAGUG-3 ′ (SEQ ID NO: 25),

5′-CAUCAGAGGAGACUGCCAGU-3 ′ (SEQ ID NO: 26),

5′-ACAUCAGAGGAGACUGCCAG-3 ′ (SEQ ID NO: 27),

5′-CACAUCAGAGGAGACUGCCA-3 ′ (SEQ ID NO: 28),

5′-CCACAUCAGAGGAGACUGCC-3 ′ (SEQ ID NO: 29),

5′-AUCAAAGGAGACUGCCGGUG-3 ′ (SEQ ID NO: 30),

5′-CAUCAAAGGAGACUGCCGGU-3 ′ (SEQ ID NO: 31),

5′-ACAUCAAAGGAGACUGCCGG-3 ′ (SEQ ID NO: 32),

5′-CACAUCAAAGGAGACUGCCG-3 ′ (SEQ ID NO: 33), or

5′-CCACAUCAAAGGAGACUGCC-3 ′ (SEQ ID NO: 34).

Still other embodiments relate to a vector encoding a guide sequence of comprising the nucleic sequence of at least one of:

5′-AUCAGAGGAGACUGCCAGUG-3 ′ (SEQ ID NO: 25),

5′-CAUCAGAGGAGACUGCCAGU-3 ′ (SEQ ID NO: 26),

5′-ACAUCAGAGGAGACUGCCAG-3 ′ (SEQ ID NO: 27),

5′-CACAUCAGAGGAGACUGCCA-3 ′ (SEQ ID NO: 28),

5′-CCACAUCAGAGGAGACUGCC-3 ′ (SEQ ID NO: 29),

5′-AUCAAAGGAGACUGCCGGUG-3 ′ (SEQ ID NO: 30),

5′-CAUCAAAGGAGACUGCCGGU-3 ′ (SEQ ID NO: 31),

5′-ACAUCAAAGGAGACUGCCGG-3 ′ (SEQ ID NO: 32),

5′-CACAUCAAAGGAGACUGCCG-3 ′ (SEQ ID NO: 33), or

5′-CCACAUCAAAGGAGACUGCC-3 ′ (SEQ ID NO: 34).

### BRIEF DESCRIPTION OF THE DRAWINGS

FIGS. 1(A-G) illustrate in vitro validation of rd12 mutation correction by the adenine base editor. A) The rd12 mouse model has a homozygous C·G to T·A nonsense mutation in exon 3 of the Rpe65 gene, changing arginine to a stop codon. B) The adenine base editor (ABE) efficiently edits “A” nucleotides in the genome that correspond to a window of positions ~4-8 in the guide RNA used to target it, counting the “NGG” PAM as positions 21-23. C) Validation of rd12 (left lane) and WT (right lane) reporter cell lines by Western blot analysis. The Western blot analysis shows the RPE65 band (65 kDa) in the WT cell line, but not in the rd12 cell line. α-tubulin (52 kDa) served as the loading control. D) Five single-guide RNAs (sgRNAs) (SEQ ID NOs: 15-19) were designed to place the target mutation (SEQ ID NO: 8) within the ABE activity window. E) The Western blot analysis of rd12 cells following ABE and sgRNA transfection shows the rescue of RPE65 protein with sgRNA-A5 and sgRNA-A6. The positive control (+ Ctrl) was rd12 cells transfected with a plasmid encoding mouse Rpe65 cDNA under a CMV promoter, and the negative control (-Ctrl) was non-transfected rd12 cells. ABE (200 kDa) expression confirmed the successful transfection. β-actin (42 kDa) served as the loading control. An additional band at 50 kDa has unknown identity but is irrelevant to the RPE65. F) Immunocytochemistry of rescued rd12 cells with sgRNA-A5 and sgRNA-A6. Rescued cells show positive immunofluorescence from the RPE65 antibody (red). GRP78, green; DAPI, blue. GRP78 served as a marker for endoplasmic reticulum localization. Scale bar, 50 µm. G, Deep sequencing of the mutation region in the rd12 cell line at 48 h post-transfection (n=4, each group). Frequencies of each allelic variant (SEQ ID NOs: 39-43) are shown as mean SEM. Types of allelic variants are shown as DNA and translated amino acid sequences in the table. Allelic variants occurring at less than 0.1% frequency were not included in the analysis.

FIGS. 2(A-G) illustrate restoration of RPE65 expression in the RPE of rd12 mice following subretinal delivery of the adenine base editor. A, Schematic maps of two lentiviral vector genomes (LV- ABE-A5 and LV-ABE-A6) for subretinal delivery and the outline of in vivo experiments and correction analysis. LV-ABE-A5 and LV-ABE-A6 express the adenine base editor (ABE) and sgRNA-A5 or sgRNA-A6. B, Western blot analysis of RPE65 (65 kDa) expression in rd12 mouse eye tissue extract following LV-ABE-A5 and LV-ABE-A6 injection. ABE (200 kDa) expression confirmed the successful transduction of lentivirus to the RPE. β-actin (42 kDa) served as the loading control. C, Immunofluorescence analysis showing the correct localization of RPE65 expression in cross-sections of eyes from treated rd12 mouse. DAPI, blue. Scale bar, 50 µm. D, Immunofluorescence analysis of RPE flatmounts from rd12 mice treated with LV- ABE-A5 and LV-ABE-A6 show RPE65-positive cells (green). ZO-1 (red), a marker for tight junction protein, demarcates the RPE cell membranes. DAPI, blue. Scale bar, 50 µm. E, Quantification of gene correction based on the percentage of area expressing RPE65 immunofluorescence in RPE flatmount from each mouse as presented in C. The percentages are shown as mean SEM. Ten random images were taken from each group for quantification. F, Deep sequencing analysis of rd12 locus in genomic DNA isolated from the RPE tissue of control (n=3), A5- and A6-treated mice (n=5 each) at 5-weeks post-injection. Frequencies of target base-edited and indel-bearing alleles are shown as mean SEM. G, Pie charts showing the composition of allelic variants in representative treated eye samples, LV-ABE-A5 (left) and LV-ABE-A6 (right). Fifteen nucleotides spanning the target rd12 mutation (SEQ ID NO: 44) is shown as a reference using the unedited rd12 mouse sequence.

FIGS. 3(A-I) illustrate evaluation of the functional rescue in rd12 mice after treatment with ABE. A, Schematic representation of the visual cycle demonstrating the enzymatic role of RPE65. In the dark, regenerated 11-cis-retinal binds the opsin (white rectangle) in an inactive conformation. Upon absorption of light, 11-cis-retinal photoisomerizes to all-trans-retinal, triggering the phototransduction process through a G-protein signaling cascade, ultimately leading to neuronal signaling. RDH5, retinol dehydrogenase 5. B, Retinoid profiles from eyes obtained from the 48 h dark-adapted and treated (LV-ABE-A5) rd12 mouse show the production of active chromophore 11-cis-retinal, which is absent in the untreated (PBS) rd12 mouse. Peak a, all-trans-retinyl esters; peak b, 11-cis-retinal. Each chromatogram represents the homogenate from two eyes. C, Retinoid profiles of the eyes following 0.5 s flash illumination reveals a partial photoisomerization of 11-cis-retinal into all-trans-retinal in both the WT and treated rd12 mouse. Neither all-trans-retinal nor 11-cis-retinal was detected from untreated rd12 mouse eyes. Peak a, all-trans-retinyl esters; peak b, 11-cis-retinal; peak c, all-trans-retinal. Each chromatogram represents the homogenate from two eyes. D, Schematic representation of the visual pathway in the mouse. In mice, visual signals from the retina are transmitted to the superior colliculus (SC) and primary visual cortex (V1), responsible for reflex-based optomotor response and cortical visual processing, respectively. Malfunction at any point along the visual pathway results in functional deficits. Electroretinography (ERG), optomotor response (OMR) tests and V1 recordings can assess the integrity of the visual pathway. E, Scotopic ERG waveforms of untreated and treated eyes from rd12 mice at light stimulus intensity of -0.3 log (cd·s/m²). A representative ERG response from one WT mouse is shown. The a-wave is a measure of the photoreceptor response; the b-wave is a measure of the inner retinal cell response. F, Scotopic a- and b-wave amplitudes of ERG responses from untreated rd12 (n = 6), treated rd12 (n = 6) and WT mice (n = 5) at light stimulus intensity of -0.3 log (cd·s/m²). Mean ± SEM are shown. *P < 0.05, **P < 0.01, ***P < 0.001, one-way ANOVA with Bonferroni test. G, Schematic drawing of the OMR apparatus. A mouse is placed on an elevated platform where it can freely move and track the virtual rotating pattern stimulus displayed on the screen. Evaluation of head movement synchronous to the stimulation is automated. H, OMR index (correct/incorrect head movements) of individual animals in each group are represented as thin dashed lines at various pattern contrast (%). Average response from each group is represented as thick lines. WT (n=5); treated rd12 (n=5); untreated rd12 (n=6). OMR index of 1 indicates head movements by chance, and statistically significant tracking was measured using one sample t-test comparing to hypothetical mean of 1. I, WT (n=5) and treated rd12 (n=5) mice exhibited statistically significant reflex-based tracking behaviors, whereas none of the untreated rd12 (n=6) mice showed consistent reflex-based visual behavior. The ambient luminance during the test was set at ~1 lux, corresponding to low twilight light level. Mean ± SEM are shown. *P < 0.05, **P < 0.01, ***P < 0.001, one-way ANOVA with Bonferroni test.

FIGS. 4(A-I) illustrate ABE treatment restores visual responses in primary visual cortex (V1) of rd12 mice. A-C, Flash-evoked responses were obtained in WT (A) and treated (LV-ABE-A5-injected) rd12 mice (C), but not untreated (PBS-injected) rd12 mice (B) as shown by raster plots for single neurons at the top and in the population averages at the bottom of each panel. Shading indicates SEM. The time of flash stimulus is represented as white horizontal bar from 0 s (ON) to 0.5 s (OFF), n = 34 neurons (WT); n = 22 neurons (untreated rd12); n = 30 neurons (treated rd12). D, The summary of population firing rate for each group in response to flash stimulus. E-I, Comparisons of single V1 neuron responses to different stimulus parameters between WT and treated rd12 animals are shown by tuning curves for orientation/direction (E), spatial frequency (F), temporal frequency (G), size (H) and contrast (I). HWHH, half-width at half height; SF, preferred spatial frequency; TF, optimal temporal frequency; size, optimal stimulus diameter; C50, percent contrast at half of the peak response. Horizontal dashed lines indicate background activity. The vertical dashed lines indicate optimal stimulus parameters. Mean ± SEM are shown. n.s., not significant; *P < 0.05, **P < 0.01, ***P < 0.001, two-tailed Mann-Whitney U-tests.

FIGS. 5(A-C) illustrate in vitro validation of HDR-mediated correction of the rd12 mutation. A, Schematic representation of sgRNAs and 140 nt single-stranded donor sequence targeting the mutation region of the Rpe65 gene in the rd12 cell line (top). SURVEYOR assay showing indel formation in the rd12 cells transiently transfected with vectors expressing sgRNA and Cas9 (bottom). Cleavage product bands are indicated with red arrow. The positive control (Pos Ctrl) is obtained from an equimolar mix of genomic DNA of WT and rd12 cell lines. The negative control (Neg Ctrl) is obtained from genomic DNA of non-transfected rd12 cell line. B, The Western blot analysis of the rd12 cells following nucleofection of Cas9, sgRNA and donor shows the RPE65 band (65 kDa). Cell lysates from the WT and rd12 cell lines served as the positive and negative control, respectively. β-actin (42 kDa) served as the loading control. C, Deep DNA sequencing of the mutation region in the rd12 cell line at 48 h post-nucleofection (n=2 per group). Mean percentages of total edited alleles, including indels and substitutions, and HDR alleles are shown in the table.

FIGS. 6(A-E) illustrates HDR-mediated rd12 mutation correction is inefficient in vivo. A, Dual AAV vector system for RPE-specific delivery of components required for HDR-mediated correction of rd12 mutation. B, Representative cross-section image of the AAV1-CMV-GFP- injected eye shows the RPE-specific tropism of AAV1 serotype at 4-weeks post-subretinal injection. ONL, outer nuclei layer; INL, inner nuclei layer; GCL, ganglion cells layer. Scale bar = 50 µm. C, Deep sequencing analysis of PCR amplicons generated across the target site in rd12 mice 6 months after dual AAV injection. The rd12 and donor template reference sequences are shown at top (3′ to 5′) (SEQ ID Nos: 45-46). The frequencies of top occurring allelic variants (SEQ ID NOs: 47-51) are shown as mean ± SEM (n=4). Only one biological sample contained 0.01% of HDR-edited allele (8 reads), which was not detected in three other samples. Red letters highlight inserted sequences. Horizontal dashed lines indicate deleted sequences. The red inverted triangles indicate the predicted cleavage site. D, Western blot analysis of the RPE extracts from WT, untreated rd12, and dual- AAV-treated rd12 mice (HDR#1, 2). The RPE65 (65 kDa) is not restored from the dual-AAV treatment although the Cas9 (150 kDa) expression is confirmed. β-actin (42 kDa) served as the loading control. E, Representative scotopic ERG waveforms of WT, untreated rd12 and dual- AAV-treated rd12 mice at light stimulus intensity of -0.3 log (cd·m⁻² ). The a-wave amplitude reflects the photoreceptor response; the b-wave amplitude reflects the inner retinal cell response. Dual-AAV-injected rd12 mice do not show any noticeable improvement in retinal function.

FIGS. 7(A-B) illustrate delivery of xABE with sgRNA-A5 or sgRNA-A6 results in restoration of RPE65 in the rd12 cell line. A, Schematic representation of two plasmids for transient transfection into the rd12 cell line. Plasmid 1 expresses the evolved adenine base editor (xABE) under a CMV promoter. Plasmid 2 expresses one of five sgRNA sequences (A4 to A8) under a U6 promoter. B, The Western blot analysis of rd12 cells following xABE and sgRNA transfection shows the rescue of RPE65 protein with sgRNA-A5 and sgRNA-A6. The positive control (+ Ctrl) was rd12 cells transfected with a plasmid encoding mouse Rpe65 cDNA under a CMV promoter, and the negative control (- Ctrl) was non-transfected rd12 cells. The xABE (200 kDa) expression confirmed the successful transfection. β-actin (42 kDa) served as the loading control. An additional band at 50 kDa has unknown identity but is irrelevant to the RPE65.

FIGS. 8(A-C) illustrates subretinal delivery of lentivirus results in efficient and RPE- specific transgene expression in mice. a, Immunofluorescence assessment of GFP expression in WT mouse eye cryosections 4 weeks after subretinal injection of lentivirus containing a CMV promoter-driven GFP gene (LV-CMV-GFP) or PBS (control). A representative LV-CMV-GFP-injected eye shows bright confluent GFP fluorescence across the greater part of the RPE layer, whereas the PBS-injected eye shows no GFP expression. Scale bar, 500 µm. B, At higher magnification, the RPE-specific transgene expression from the lentivirus can be observed. Scale bar, 50 µm. C, Immunofluorescence assessment of GFP expression in the retinal and RPE wholemounts from a mouse eye injected with LV-CMV-GFP. Images taken with phase-contrast (left) and FITC (right) filters are shown. GFP expression is primarily confined to the RPE tissue, except a minor contamination on the retinal layer resulting from the dissection. Scale bar, 1 mm.

FIGS. 9(A-C) illustrate comparison of visually evoked potentials recorded from WT, treated and untreated rd12 mice. A, Representative examples of single visually evoked potentials (VEPs) recorded from a WT, treated rd12 and untreated rd12 mouse. B, Mean VEPs for each studied group averaged by number (n) of recording sites. Shading indicates SEM. N, number of mice. C, Population summary of average VEP amplitudes as shown in (B). d, Average response latency for each group. Data are shown as mean ± SEM. n.s., not significant; *P < 0.05, **P < 0.01, ***P < 0.001, two-tailed Mann-Whitney U-tests.

FIGS. 10(A-F) illustrate a summary of V1 neuron population responses to various parameters of drifting sine wave gratings. The V1 neuron population responses of WT and treated rd12 mice are measured with different parameters. A, The average orientation/direction tuning width measured in degrees at the half-width at half-height (HWHH). B, preferred spatial frequency (SF). C, optimal temporal frequency (TF). D, optimal stimulus diameter (size). E, percent contrast at half of the peak response (C50). F, background activity. n=117 neurons from 5 treated rd12 mice, n=106 neurons from 6 WT mice. Data are shown as mean ± SEM. n.s., not significant; *P < 0.05, **P < 0.01, ***P < 0.001, two-tailed Mann-Whitney U-tests.

FIGS. 11(A-D) illustrate early cone dysfunction and degeneration in the rd12 mouse due to a loss-of-function mutation in Rpe65. (A), Rd12 mouse has an inherent nonsense mutation (C·G to T·A) in the exon 3 of Rpe65 gene, resulting in truncated, nonfunctional RPE65 protein. Deficiency of functional RPE65 in rd12 mouse impairs the production of 11-cis-retinal (11cRAL) by a blockade of conversion from all-trans-retinyl ester (atRE) into 11-cis-retinol (11cROL) and contributes to early cone cell death. (B), The retinal flatmounts from a 3-week-old and 6-week-old wild-type (upper) and rd12 (lower) mice, labeled with M-opsin and S-opsin antibodies. Retina is oriented with dorsal towards the top and ventral towards the bottom. Scale bar, 1 mm. (C), The retinal cryosections representing the dorsal region from a 6-week-old WT (left), 3-week-old rd12 (middle) and 6-week-old rd12 (right) mouse, labeled with M-opsin antibody. (D), The retinal cryosections representing the ventral region from the same eyes in (C), labeled with S-opsin antibody. DAPI. OS, outer segment; IS, inner segment; ONL, outer nuclear layer. Scale bar, 20 µm.

FIGS. 12(A-H) illustrate in vitro screening and in vivo validation of enhanced base editing with NG-ABE and sgRNA A6. (A), Schematic representation of the in vitro strategy to screen different adenine base editors (ABEs) and sgRNAs capable of correcting the rd12 mutation in the mutant cell line. Table on the right summarizes the choice of ABE and sgRNA for each transfection. (B), Heatmap shows the average frequency of A-to-G conversion in the genomic DNA isolated from the transfected cells. The rd12 mutation is labeled in red. n = 3, mean ± SD. (C), Percentage of RPE65 alleles that have precise correction or contain bystander editing from each transfection group. n = 3, mean ± SD. (D), Map of lentivirus (LV-NG-ABE-A6) for subretinal delivery of NG-ABE and sgRNA-A6 to the rd12 mouse, and the experimental timeline. (E), Frequency of A-to-G conversion in the RPE65 cDNA isolated from lentivirus-injected (ABE-treated) and PBS-injected (untreated) rd12 mouse eyes. The bottom sequence represents 20-nucleotide sgRNA-A6 (SEQ ID NO: 52) with the targeted mutation highlighted. ABE-treated, n = 6; Untreated, n = 3. Mean ± SD. (F), Frequency of precisely corrected, functional RPE65 transcripts from the groups in (E). (G), Average composition of RPE65 transcript variants in each eye from the untreated (n = 3), WT (n = 3) and ABE-treated (n = 6) mice. Mean ± SD. (H), Frequency of A-to-G conversion at ten potential off-target sites identified by CIRCLE-seq. n = 3, each group. Mean ± SD.

FIGS. 13(A-E) illustrate rescue of cone-mediated visual function in rd12/gnat1^{-/-} mice after ABE treatment. (A), Breeding strategy to generate a homozygous rd12/Gnat1^{-/-} mouse line. Knockout (KO) of Gnat1 abolishes the phototransduction signaling cascade from rods, leaving only cone-mediated phototransduction. (B), Progression of cone degeneration in the rd12/Gnat] ^{-/-} mouse from 2 weeks of age to 8 weeks of age. M-opsin, green. S-opsin. Scale bar, 1 mm. (C), Experimental timeline to evaluate the cone function and survival in rd12/Gnat]^{-/-} mice after ABE treatment. (D), Representative photopic ERG waveforms of M-cones (top) and the average b-wave amplitudes (bottom), evoked with the indicated green light flashes, in treated and untreated rd12/Gnat1^{-/-} mice (n = 8, each group). (E), Representative photopic ERG waveforms of S-cones (top) and the average b-wave amplitudes (bottom), evoked with the indicated UV light flashes, in treated and untreated rd12/Gnat1^{-/-}mice (n = 8, each group). Mean ± SD. ***, P < 0.001; two-tailed Mann-Whitney U-test.

FIGS. 14(A-F) illustrate ABE treatment restores the visual pathway from cones to the visual cortex. (A), Representative visually evoked potentials (VEPs) from a control Gnat1^{-/-}treated rd12/Gnat1^{-/-}, and untreated rd12/Gnat1^{-/-} mouse. (B), Population average of VEPs recorded from three groups in (A). N, total number of mice; n, total number of recording sites from each experimental group. (C), VEP amplitudes recorded from three groups. (D), Response latencies of VEPs recorded from three groups. *, P < 0.05; **, P < 0.01; ***, P < 0.001; two-tailed Mann-Whitney U-test. (E), Raster plots and corresponding histograms for single neurons in Gnat^{-/-} (n = 387 cells)⁻, treated (n = 286 cells) and untreated rd12/Gnat1^{-/-} (n = 121 cells) mice, respectively. (F), Population average histograms for the same groups as (E).

FIGS. 15(A-E) illustrate protection of cone photoreceptors and correct opsin localization in 2-month-old rd12/Gnat1^{-/-} mice following ABE treatment. (A), Representative retinal flatmounts from untreated (left) and treated (right) rrd12/Gnat1^{-/-} mouse eyes, labeled with M-opsinand S-opsin antibodies. Scale bar, 1 mm. (B), Magnified view of M-cones in dorsal retina from untreated and treated rd12/Gnat1^{-/-} mice. Scale bar, 50 µm. (C), Magnified view of S-cones in ventral retina from untreated and treated rd12/Gnat1⁻^{/-} mice. Scale bar, 50 µm. (D), Quantification of M-cones and S-cones in each quadrant as shown in (B) and (C), at dorsal and ventral retina 1 mm away from the optic nerve. Five quadrants across the dorsal or ventral retina were analyzed from each eye, with a total of 20 quadrants from 4 eyes per group. (E), Retinal cryosections from Gnat1^{-/-}and untreated and treated rd12/Gnat1^{-/-} mice, labeled with M-opsin (top) and S-opsin (bottom). DAPI; OS, outer segment; IS, inner segment; ONL, outer nuclear layer. Scale bar, 20 µm.

FIGS. 16(A-E) illustrate single-cell RNA-seq of ABE-treated retinas reveals the rescue of genes associated with phototransduction and cone survival. (A), UMAP representation of the single-cell RNA-seq dataset colored by annotated cell type. (B), UMAP plots showing the distribution of single cells of 2-month-old WT, and untreated and treated rd12 retina samples (n = 4, each group). (C), Violin plot profiles of S-opsin (Opn1sw) and M-opsin (Opn1mw) expression levels in individual cone cells. (D), Violin plot profiles of cone phototransduction-associated expression levels in individual cone cells. ***, P < 0.001; Wilcox test in Seurat. (E), Retinal cryosections showing the expression of cone-arrestin in 2-month-old WT, and untreated and treated rd12 mice. Cone cells are labeled with peanut agglutinin (PNA;). DAPI Scale bar, 20 µm.

FIGS. 17(A-B) illustrate base editing analysis of Rpe65 in mouse RPE genomic DNA following treatment. (A), Frequency of A-to-G conversion in the Rpe65 gDNA isolated from lentivirus-injected (ABE-treated) and PBS-injected (untreated) rd12 mouse eyes. Bottom sequence represents 20-nucleotide sgRNA-A6 (SEQ ID NO: 52) with the targeted mutation highlighted in red. ABE-treated, n = 6; Untreated, n = 3. Mean ± SD. (B), Frequency of precisely corrected, functional Rpe65 alleles from the same eyes in (A).

FIGS. 18(A-B) illustrate functional RPE65 rescue in rd12 mice treated with LV-NG-ABE-A6. (A) Immunoblot showing restoration of a full-length RPE65 (65 kDa) protein in ABE-treated rd12 mouse RPE tissue lysate. ABE (200 kDa), base editor; β-actin (42 kDa), loading control. (B) Scotopic a-wave and b-wave amplitudes evoked with light stimulus of -0.3 log (cd·s/m²) from age-matched WT, untreated and treated rd12 mice (n = 8 eyes each group).

FIGS. 19(A-C) illustrate use of dual-AAV vectors for split base editor delivery. (A), Schematic of split intein-AAV vectors. (B), Scotopic ERG a-wave and b-wave amplitudes recorded at three timepoints in AAV-injected rd12 mice (n = 5 eyes). (C), Frequency of A-to-G conversion in the Rpe65 gDNA isolated from AAV-injected and untreated rd12 mouse eyes. Bottom sequence represents 20-nucleotide sgRNA-A6 (SEQ ID NO. 52) with the targeted mutation. ABE-treated, n = 5; Untreated, n = 3. Mean ± SD.

FIGS. 20(A-C) illustrate representative retinal flatmount of 2-month-old Gnat1^{-/-} mouse. (A), Overall view of the retinal flatmount from 2-month-old Gnat1^{-/-} mouse, labelled with M-opsin and S-opsin antibodies. Scale bar, 1 mm. (B), Magnified view of M-cones labeled with M-opsin antibody at dorsal retina. Scale bar, 50 µm. (C), Magnified view of S-cones labeled with S-opsin antibody at ventral retina. Scale bar, 50 µm.

FIGS. 21(A-B) illustrate a comparison of photopic ERG b-wave amplitudes against Gnat1^{-/-} mice. (A), Photopic b-wave amplitudes evoked with green light flashes in untreated, treated rd12/Gnat1^{-/-} and Gnat1^{-/-}. (B), Photopic b-wave amplitudes evoked with UV light flashes in untreated, treated rd12/Gnat1^{-/-} and Gnat1^{-/-}. n = 8, each group. Mean ± SD. ***, P < 0.001; two-tailed Mann-Whitney U-test.

FIG. 22 illustrates long-term protection of cone function and structure in 6-month-old rd12/Gnat1^{-/-} mice by ABE treatment. (A), Amplitudes of photopic ERG b-waves of M-cones recorded from the same eyes at 2 months and 6 months of age (n = 4 eyes). (B), Amplitudes of photopic ERG b-waves of S-cones recorded from the same eyes at 2 months and 6 months of age (n = 4 eyes). (C), Representative retinal flatmounts from 6-month-old treated (left) and untreated (right) rd12; Gnat1^{-/-}, labeled with M-opsin and S-opsin antibodies. Scale bar, 1 mm. (D), Magnified view of M-cones (green) in dorsal, and S-cones in ventral retina from treated and untreated rd12; Gnat1^{-/-} mice. Scale bar, 50 µm. (E), Quantification of M-cones (upper) and S-cones (lower) in each quadrant, as shown in (D), at dorsal and ventral retina 1 mm away from the optic nerve. Five quadrants across the dorsal or ventral retina were analyzed from one eye, with a total of 15 quadrants from 3 eyes per group.

### DEFINITIONS

As used herein and in the claims, the singular forms “a,” “an,” and “the” include the singular and the plural unless the context clearly indicates otherwise. Thus, for example, a reference to “an agent” includes a single agent and a plurality of such agents.

The term “deaminase” or “deaminase domain” refers to a protein or enzyme that catalyzes a deamination reaction. In some embodiments, the deaminase is an adenosine deaminase, which catalyzes the hydrolytic deamination of adenine or adenosine. In some embodiments, the deaminase or deaminase domain is an adenosine deaminase, catalyzing the hydrolytic deamination of adenosine or deoxy adenosine to inosine or deoxyinosine, respectively. In some embodiments, the adenosine deaminase catalyzes the hydrolytic deamination of adenine or adenosine in deoxyribonucleic acid (DNA). The adenosine deaminases (e.g., engineered adenosine deaminases, evolved adenosine deaminases) provided herein may be from any organism, such as a bacterium. In some embodiments, the deaminase or deaminase domain is a variant of a naturally-occurring deaminase from an organism. In some embodiments, the deaminase or deaminase domain does not occur in nature.

As used herein, an “adenosine deaminase” is an enzyme that catalyzes the deamination of adenosine, converting it to the nucleoside inosine. Under standard Watson-Crick hydrogen bond pairing, an adenosine base hydrogen bonds to a thymine base (or an uracil in case of RNA). When adenine is converted to inosine, the inosine undergoes hydrogen bond pairing with cytosine. Thus, a conversion of “A” to inosine by adenosine deaminase will cause the insertion of “C” instead of a “T” during cellular repair and/or replication processes. Since the cytosine “C” pairs with guanine “G”, the adenosine deaminase in coordination with DNA replication causes the conversion of an A T pairing to a C-G pairing in the double- stranded DNA molecule.

As used herein, “base editing” is a genome editing technology that involves the conversion of a specific nucleic acid base into another at a targeted genomic locus. In certain aspects, this can be achieved without requiring double-stranded DNA breaks (DSB).

The term “base editors (BEs)” or “nucleobase editors (NBEs)” as used herein, refers to an agent comprising a polypeptide that is capable of making a modification to a base (e.g., A, T, C, G, or U) within a nucleic acid sequence (e.g., DNA or RNA), for example, any of the Cas9 fusion proteins provided herein. In some embodiments, the base editor is capable of deaminating a base within a nucleic acid. In some embodiments, the base editor is capable of deaminating a base within a DNA molecule. In some embodiments, the base editor is capable of deaminating an adenine (A) in DNA. In some embodiments, the base editor is a fusion protein comprising a nucleic acid programmable DNA binding protein (napDNAbp) fused to an adenosine deaminase. In some embodiments, the base editor is a Cas9 protein fused to an adenosine deaminase. In some embodiments, the base editor is a Cas9 nickase (nCas9) fused to an adenosine deaminase. In some embodiments, the base editor is a nuclease-inactive Cas9 (dCas9) fused to an adenosine deaminase. In some embodiments, the fusion protein comprises a nuclease-inactive Cas9 (dCas9) fused to a deaminase which still binds DNA in a guide RNA-programmed manner via the formation of an R-loop, but does not cleave the DNA backbone. In some embodiments, the fusion protein comprises a Cas9 or Cas9 nickase (nCas9) fused to an adenosine deaminase. Base editors comprising an adenosine deaminase (e.g., adenosine base editors) have been described in PCT/US2017/045381 (published as WO 2018/027078); PCT/US2018/056146 (published as WO 2019/079347); and PCT/2019/033848; the entire contents of each of which are incorporated herein by reference. Exemplary adenosine base editors include, without limitation xCas9-3.7-ABE (xABE). Other base editors include cytidine base editors, which, in some embodiments, are fusion proteins comprising a Cas9 nickase fused to a deaminase, e.g., a cytidine deaminase (rAPOBECl) which converts a DNA base cytosine to uracil. One such base editor is referred to as “BE1” in the literature. In some embodiments, the fusion protein comprises a nuclease-inactive Cas9 fused to a deaminase and further fused to a UGI domain (uracil DNA glycosylase inhibitor, which prevents the subsequent U:G mismatch from being repaired back to a C:G base pair). One such base editor is referred to as “BE2” in the literature.

The terms “nucleobase editors (NBEs)” and “base editors (BEs)” may be used interchangeably. The term “base editors” encompasses any base editor known or described in the art at the time of this filing, but also the improved base editors.

The term “Cas9” or “Cas9 nuclease” or “Cas9 moiety” refers to a CRISPR associated protein 9, or functional fragment thereof, and embraces any naturally occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. More broadly, a Cas9 is a type of “RNA-programmable nuclease” or “RNA-guided nuclease” or more broadly a type of “nucleic acid programmable DNA binding protein (napDNAbp)”. The term Cas9 is not meant to be particularly limiting and may be referred to as a “Cas9 or equivalent.” Examples of Cas9 proteins are further described herein and/or are described in the art and are incorporated herein by reference. The present disclosure is unlimited with regard to the particular Cas9 that is employed in the improved base editors of the invention.

As used herein, the term “CRISPR” refers to a family of DNA sequences (e.g., CRISPR clusters) in bacteria and archaea that represent snippets of prior infections by a virus that have invaded the prokaryote. The snippets of DNA are used by the prokaryotic cell to detect and destroy DNA from subsequent attacks by similar viruses and effectively compose, along with an array of CRISPR-associated proteins (including Cas9 and homologs thereof) and CRISPR-associated RNA, a prokaryotic immune defense system. In nature, CRISPR clusters are transcribed and processed into CRISPR RNA (crRNA). In certain types of CRISPR systems (e.g., type II CRISPR systems), correct processing of pre-crRNA requires a trans-encoded small RNA (tracrRNA), endogenous ribonuclease 3 (rnc) and a Cas9 protein. The tracrRNA serves as a guide for ribonuclease 3-aided processing of pre-crRNA.

Subsequently, Cas9/crRNA/tracrRNA endonucleolytically cleaves linear or circular dsDNA target complementary to the RNA. Specifically, the target strand not complementary to crRNA is first cut endonucleolytically, then trimmed 3′-5′ exonucleolytically. In nature, DNA-binding and cleavage typically requires protein and both RNAs. However, single guide RNAs (“sgRNA”, or simply “gNRA”) can be engineered so as to incorporate aspects of both the crRNA and tracrRNA into a single RNA species - the guide RNA. See, e.g., Jinek M., Chylinski K., Fonfara I., Hauer M., Doudna J.A., Charpentier E. Science 337:816-821(2012), the entire contents of which is hereby incorporated by reference. Cas9 recognizes a short motif in the CRISPR repeat sequences (the PAM or protospacer adjacent motif) to help distinguish self versus non-self. CRISPR biology, as well as Cas9 nuclease sequences and structures are well known to those of skill in the art (see, e.g., “Complete genome sequence of an Ml strain of Streptococcus pyogenes.” Ferretti et al, J.J., McShan W.M., Ajdic D.J., Savic D.J., Savic G., Lyon K., Primeaux C., Sezate S., Suvorov A.N., Kenton S., Lai H.S., Lin S.P., Qian Y., Jia H.G., Najar F.Z., Ren Q., Zhu H., Song L., White J., Yuan X., Clifton S.W., Roe B.A., McLaughlin R.E., Proc. Natl. Acad. Sci. U.S.A. 98:4658-4663(2001); “CRISPR RNA maturation by trans-encoded small RNA and host factor RNase III.” Deltcheva E., Chylinski K., Sharma C.M., Gonzales K., Chao Y., Pirzada Z.A., Eckert M.R., Vogel J., Charpentier E., Nature 471:602-607(2011); and “A programmable dual-RNA- guided DNA endonuclease in adaptive bacterial immunity.” Jinek M., Chylinski K., Fonfara L, Hauer M., Doudna J.A., Charpentier E. Science 337:816-821(2012), the entire contents of each of which are incorporated herein by reference). Cas9 orthologs have been described in various species, including, but not limited to, S. pyogenes and S. thermophilus. Additional suitable Cas9 nucleases and sequences will be apparent to those of skill in the art based on this disclosure, and such Cas9 nucleases and sequences include Cas9 sequences from the organisms and loci disclosed in Chylinski, Rhun, and Charpentier, “The tracrRNA and Cas9 families of type II CRISPR-Cas immunity systems” (2013) RNA Biology 10:5, 726-737; the entire contents of which are incorporated herein by reference.

As used herein, the term “deaminase” or “deaminase domain” or “deaminase moiety” refers to a protein or enzyme that catalyzes a deamination reaction. In some embodiments, the deaminase is an adenosine deaminase, which catalyzes the hydrolytic deamination of adenine or adenosine (e.g., an engineered adenosine deaminase that deaminates adenosine in DNA). In some embodiments, the deaminase or deaminase domain is a cytidine deaminase, catalyzing the hydrolytic deamination of cytidine or deoxycytidine to uridine or deoxyuridine, respectively. In some embodiments, the deaminase or deaminase domain is a cytidine deaminase domain, catalyzing the hydrolytic deamination of cytosine to uracil. In some embodiments, the deaminase or deaminase domain is a naturally-occurring deaminase from an organism, such as a human, chimpanzee, gorilla, monkey, cow, dog, rat, or mouse. In some embodiments, the deaminase or deaminase domain is a variant of a naturally-occurring deaminase from an organism that does not occur in nature. For example, in some embodiments, the deaminase or deaminase domain is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring deaminase from an organism. The term deaminase also embraces any genetically engineered deaminase that may comprise genetic modifications (e.g., one or more mutations) that results in a variant deaminase having an amino acid sequence comprising one or more changes relative to a wildtype counterpart deaminase. Examples of deaminases (e.g., adenosine deaminases) are provided herein, and the term is not meant to be limiting.

The term “effective amount,” as used herein, refers to an amount of a biologically active agent that is sufficient to elicit a desired biological response. For example, in some embodiments, an effective amount of a base editor may refer to the amount of the base editor that is sufficient to edit a target site nucleotide sequence, e.g., a genome. In some embodiments, an effective amount of a base editor provided herein, e.g., of a fusion protein comprising a Cas9 and a nucleic acid editing domain (e.g., an adenosine deaminase domain) may refer to the amount of the fusion protein that is sufficient to induce editing of a target site specifically bound and edited by the fusion protein. As will be appreciated by the skilled artisan, the effective amount of an agent, e.g., a fusion protein, a nuclease, a deaminase, a hybrid protein, a protein dimer, a complex of a protein (or protein dimer) and a polynucleotide, or a polynucleotide, may vary depending on various factors as, for example, on the desired biological response, e.g., on the specific allele, genome, or target site to be edited, on the cell or tissue being targeted, and on the agent being used.

As used herein, the term “isolated protein” or “isolated nucleic acid” refers to a protein or nucleic acid that by virtue of its origin or source of derivation is not associated with naturally associated components that accompany it in its native state; is substantially free of other proteins or nucleic acids from the same species; is expressed by a cell from a different species; or does not occur in nature. Thus, a polypeptide or nucleic acid that is chemically synthesized or synthesized in a cellular system different from the cell from which it naturally originates will be “isolated” from its naturally associated components. A protein or nucleic acid may also be rendered substantially free of naturally associated components by isolation, using protein purification techniques well known in the art. In some embodiments, a protein is isolated if it makes up at least 70%, 75%, 80%, 85%, 90%, 95%, 98%, or 99% of the proteins in an isolate. In some embodiments, a nucleic acid is isolated if it makes up at least 70%, 75%, 80%, 85%, 90%, 95%, 98%, or 99% of the nucleic acids in an isolate.

The term “linker,” as used herein, refers to a chemical group or a molecule linking two molecules or moieties, e.g., a binding domain and a cleavage domain of a nuclease. In some embodiments, a linker joins a gRNA binding domain of an RNA-programmable nuclease and the catalytic domain of a deaminase. In some embodiments, a linker joins a Cas9 and base editor moiety (e.g., an adenosine deaminase). Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety.

The term “mutation,” as used herein, refers to a substitution of a residue within a sequence, e.g., a nucleic acid or amino acid sequence, with another residue, or a deletion or insertion of one or more residues within a sequence. Mutations are typically described herein by identifying the original residue followed by the position of the residue within the sequence and by the identity of the newly substituted residue. Various methods for making the amino acid substitutions (mutations) provided herein are well known in the art, and are provided by, for example, Green and Sambrook, Molecular Cloning: A Laboratory Manual (4^{th} ed., Cold Spring Harbor Laboratory Press, Cold Spring Harbor, N.Y. (2012)).

Mutations can include a variety of categories, such as single base polymorphisms, microduplication regions, indel, and inversions, and is not meant to be limiting in any way. One such example of a mutation is a nonsense mutation in the RPE65 gene on exon 3 (c. 130 C>T; p.R44X), which occurs in the an rd12 mouse model that abolishes the expression of RPE65, a key isomerase in the classical visual cycle that generates active visual chromophore, 11-cis-retinal and is used as model of Leber congenital amaurosis (LCA).

Mutations also embrace “gain-of-function” mutations, which is one which confers an abnormal activity on a protein or cell that is otherwise not present in a normal condition. Many gain-of-function mutations are in regulatory sequences rather than in coding regions, and can therefore have a number of consequences. For example, a mutation might lead to one or more genes being expressed in the wrong tissues, these tissues gaining functions that they normally lack. Alternatively, the mutation could lead to overexpression of one or more genes involved in control of the cell cycle, thus leading to uncontrolled cell division and hence to cancer. Because of their nature, gain-of-function mutations are usually dominant.

The terms “non-naturally occurring” or “engineered” are used interchangeably and indicate the involvement of the hand of man. The terms, when referring to nucleic acid molecules or polypeptides (e.g., Cas9 or deaminases) mean that the nucleic acid molecule or the polypeptide is at least substantially free from at least one other component with which they are naturally associated in nature and/or as found in nature (e.g., an amino acid sequence not found in nature). These terms also embrace nucleic acid molecules and polypeptides that have been altered (e.g., mutated), such that they are different from nucleic acid molecules or polypeptides that occur in nature.

The terms “nucleic acid” and “nucleic acid molecule,” as used herein, refer to a compound comprising a nucleobase and an acidic moiety, e.g., a nucleoside, a nucleotide, or a polymer of nucleotides. Typically, polymeric nucleic acids, e.g., nucleic acid molecules comprising three or more nucleotides are linear molecules, in which adjacent nucleotides are linked to each other via a phosphodiester linkage. In some embodiments, “nucleic acid” refers to individual nucleic acid residues (e.g., nucleotides and/or nucleosides). In some embodiments, “nucleic acid” refers to an oligonucleotide chain comprising three or more individual nucleotide residues.

The term “nucleic acid programmable DNA/RNA binding protein (napDNA/RNAbp)” refers to any protein that may associate (e.g., form a complex) with one or more nucleic acid molecules (i.e., which may broadly be referred to as a “napDNA/RNAbp -programming nucleic acid molecule” and includes, for example, a guide RNA in the case of Cas systems) which direct or otherwise program the protein to localize to a specific target nucleotide sequence (e.g., a gene locus of a genome) that is complementary to the one or more nucleic acid molecules (or a portion or region thereof) associated with the protein, thereby causing the protein to bind to the nucleotide sequence at the specific target site. This term napDNA/RNAbp embraces CRISPR Cas 9 proteins, as well as Cas9 equivalents, homologs, orthologs, or paralogs, whether naturally occurring or non-naturally occurring (e.g., engineered or recombinant), and may include a Cas9 equivalent from any type of CRISPR system. However, the nucleic acid programmable DNA binding protein (napDNAbp) that may be used in connection with this invention are not limited to CRISPR-Cas systems. The invention embraces any such programmable protein, such as the Argonaute protein from Natronobacterium gregoryi (NgAgo) which may also be used for DNA-guided genome editing. NgAgo-guide DNA system does not require a PAM sequence or guide RNA molecules, which means genome editing can be performed simply by the expression of generic NgAgo protein and introduction of synthetic oligonucleotides on any genomic sequence. See Gao F, Shen XZ, Jiang F, Wu Y, Han C. DNA-guided genome editing using the Natronobacterium gregoryi Argonaute. Nat Biotechnol 2016; 34(7):768-73, which is incorporated herein by reference.

The term “napDNA/RNAbp-programming nucleic acid molecule” or equivalently “guide sequence” refers the one or more nucleic acid molecules which associate with and direct or otherwise program a napDNA/RNAbp to localize to a specific target nucleotide sequence (e.g., a gene locus of a genome) that is complementary to the one or more nucleic acid molecules (or a portion or region thereof) associated with the protein, thereby causing the napR/DNAbp protein to bind to the nucleotide sequence at the specific target site. A non-limiting example is a guide RNA of a Cas protein of a CRISPR-Cas genome editing system.

As used herein, the term “nuclear localization signal or sequence” or “NLS” is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. Thus, a single nuclear localization signal can direct the entity with which it is associated to the nucleus of a cell. Such sequences can be of any size and composition, for example more than 25, 25, 15, 12, 10, 8, 7, 6, 5 or 4 amino acids, but will preferably comprise at least a four to eight amino acid sequence known to function as a nuclear localization signal (NLS).

The term, as used herein, “nucleobase modification moiety” or equivalently a “nucleic acid effector domain” embraces any protein, enzyme, or polypeptide (or functional fragment thereof) which is capable of modifying a DNA or RNA molecule. Nucleobase modification moieties can be naturally occurring, or can be recombinant. For example, a nucleobase modification moiety can include one or more DNA repair enzymes, for example, and an enzyme or protein involved in base excision repair (BER), nucleotide excision repair (NER), homology-dependent recombinational repair (HR), non-homologous end-joining repair (NHEJ), microhomology end-joining repair (MMEJ), mismatch repair (MMR), direct reversal repair, or other known DNA repair pathway. A nucleobase modification moiety can have one or more types of enzymatic activities, including, but not limited to endonuclease activity, polymerase activity, ligase activity, replication activity, proofreading activity.

Nucleobase modification moieties can also include DNA or RNA-modifying enzymes and/or mutagenic enzymes, such as DNA methylases and deaminating enzymes (i.e., deaminases, including cytidine deaminases and adenosine deaminases, all defined above), which deaminate nucleobases leading in some cases to mutagenic corrections by way of normal cellular DNA repair and replication processes. The “nucleic acid effector domain” (e.g., a DNA effector domain or an RNA effector domain) as used herein may also refer to a protein or enzyme capable of making one or more modifications (e.g., deamination of a cytidine residue) to a nucleic acid (e.g., DNA or RNA). Exemplary nucleic acid editing domains include, but are not limited to a deaminase, a nuclease, a nickase, a recombinase, a methyltransferase, a methylase, an acetylase, an acetyltransferase, a transcriptional activator, or a transcriptional repressor domain.

As used herein, the terms “oligonucleotide” and “polynucleotide” can be used interchangeably to refer to a polymer of nucleotides (e.g., a string of at least three nucleotides). In some embodiments, “nucleic acid” encompasses RNA as well as single and/or double-stranded DNA. Nucleic acids may be naturally occurring, for example, in the context of a genome, a transcript, an mRNA, tRNA, rRNA, siRNA, snRNA, a plasmid, cosmid, chromosome, chromatid, or other naturally occurring nucleic acid molecule. On the other hand, a nucleic acid molecule may be a non-naturally occurring molecule, e.g., a recombinant DNA or RNA, an artificial chromosome, an engineered genome, or fragment thereof, or a synthetic DNA, RNA, DNA/RNA hybrid, or including non-naturally occurring nucleotides or nucleosides. Furthermore, the terms “nucleic acid,” “DNA,” “RNA,” and/or similar terms include nucleic acid analogs, e.g., analogs having other than a phosphodiester backbone. Nucleic acids can be purified from natural sources, produced using recombinant expression systems and optionally purified, chemically synthesized, etc. Where appropriate, e.g., in the case of chemically synthesized molecules, nucleic acids can comprise nucleoside analogs such as analogs having chemically modified bases or sugars, and backbone modifications. A nucleic acid sequence is presented in the 5′ to 3′ direction unless otherwise indicated. In some embodiments, a nucleic acid is or comprises natural nucleosides (e.g., adenosine, thymidine, guanosine, cytidine, uridine, deoxyadenosine, deoxythymidine, deoxyguanosine, and deoxycytidine); nucleoside analogs (e.g., 2-aminoadenosine, 2- thiothymidine, inosine, pyrrolo-pyrimidine, 3-methyl adenosine, 5-methylcytidine, 2- aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7- deazaguanosine, 8-oxoadenosine, 8-oxoguanosine, 0(6)-methylguanine, and 2-thiocytidine); chemically modified bases; biologically modified bases (e.g., methylated bases); intercalated bases; modified sugars (e.g., 2′-fluororibose, ribose, 2′-deoxyribose, arabinose, and hexose); and/or modified phosphate groups (e.g., phosphorothioates and 5′-N-phosphoramidite linkages).

The terms “protein,” “peptide,” and “polypeptide,” are used interchangeably herein, and refer to a polymer of amino acid residues linked together by peptide (amide) bonds. The terms refer to a protein, peptide, or polypeptide of any size, structure, or function. Typically, a protein, peptide, or polypeptide will be at least three amino acids long. A protein, peptide, or polypeptide may refer to an individual protein or a collection of proteins. One or more of the amino acids in a protein, peptide, or polypeptide may be modified, for example, by the addition of a chemical entity such as a carbohydrate group, a hydroxyl group, a phosphate group, a famesyl group, an isofamesyl group, a fatty acid group, a linker for conjugation, functionalization, or other modification, etc. A protein, peptide, or polypeptide may also be a single molecule or may be a multi-molecular complex. A protein, peptide, or polypeptide may be just a fragment of a naturally occurring protein or peptide. A protein, peptide, or polypeptide may be naturally occurring, recombinant, or synthetic, or any combination thereof. The term “fusion protein” as used herein refers to a hybrid polypeptide which comprises protein domains from at least two different proteins. One protein may be located at the amino-terminal (N-terminal) portion of the fusion protein or at the carboxy-terminal (C-terminal) protein thus forming an “amino-terminal fusion protein” or a “carboxy-terminal fusion protein,” respectively. A protein may comprise different domains, for example, a nucleic acid binding domain (e.g., the gRNA binding domain of Cas9 that directs the binding of the protein to a target site) and a nucleic acid cleavage domain or a catalytic domain of a recombinase. In some embodiments, a protein comprises a proteinaceous part, e.g., an amino acid sequence constituting a nucleic acid binding domain, and an organic compound, e.g., a compound that can act as a nucleic acid cleavage agent. In some embodiments, a protein is in a complex with, or is in association with, a nucleic acid, e.g., RNA. Any of the proteins provided herein may be produced by any method known in the art. For example, the proteins provided herein may be produced via recombinant protein expression and purification, which is especially suited for fusion proteins comprising a peptide linker. Methods for recombinant protein expression and purification are well known, and include those described by Green and Sambrook, Molecular Cloning: A Laboratory Manual (4th ed., Cold Spring Harbor Laboratory Press, Cold Spring Harbor, N.Y. (2012)), the entire contents of which are incorporated herein by reference. It should be appreciated that the disclosure provides any of the polypeptide sequences provided herein without an N-terminal methionine (M) residue.

The term “recombinant” as used herein in the context of proteins or nucleic acids refers to proteins or nucleic acids that do not occur in nature, but are the product of human engineering. For example, in some embodiments, a recombinant protein or nucleic acid molecule comprises an amino acid or nucleotide sequence that comprises at least one, at least two, at least three, at least four, at least five, at least six, or at least seven mutations as compared to any naturally occurring sequence.

The term “RNA-programmable nuclease,” and “RNA-guided nuclease” are used interchangeably herein and refer to a nuclease that forms a complex with (e.g., binds or associates with) one or more RNA that is not a target for cleavage (e.g., a Cas9 or homolog or variant thereof). In some embodiments, an RNA-programmable nuclease, when in a complex with an RNA, may be referred to as a nuclease:RNA complex. Typically, the bound RNA(s) is referred to as a guide RNA (gRNA). gRNAs can exist as a complex of two or more RNAs, or as a single RNA molecule. gRNAs that exist as a single RNA molecule may be referred to as single-guide RNAs (sgRNAs), though “gRNA” is used interchangeabley to refer to guide RNAs that exist as either single molecules or as a complex of two or more molecules.

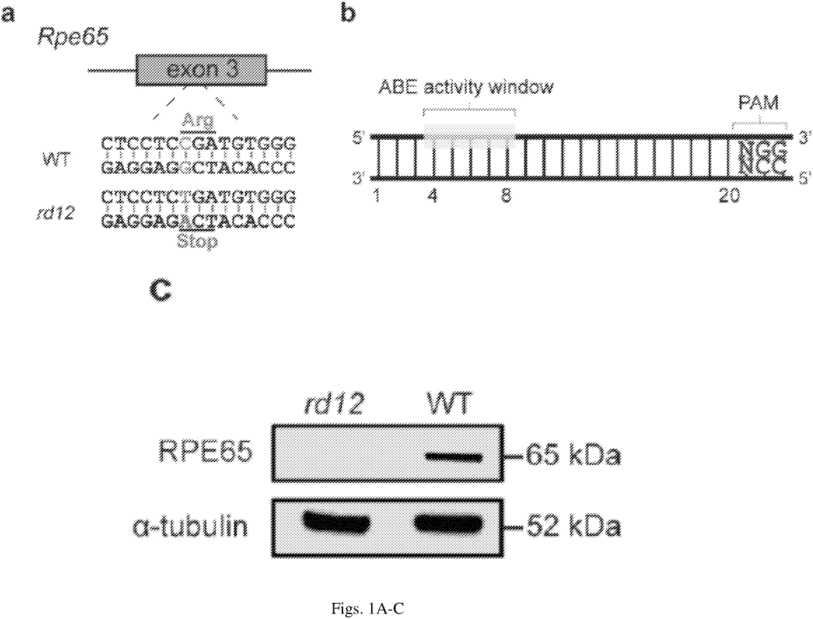

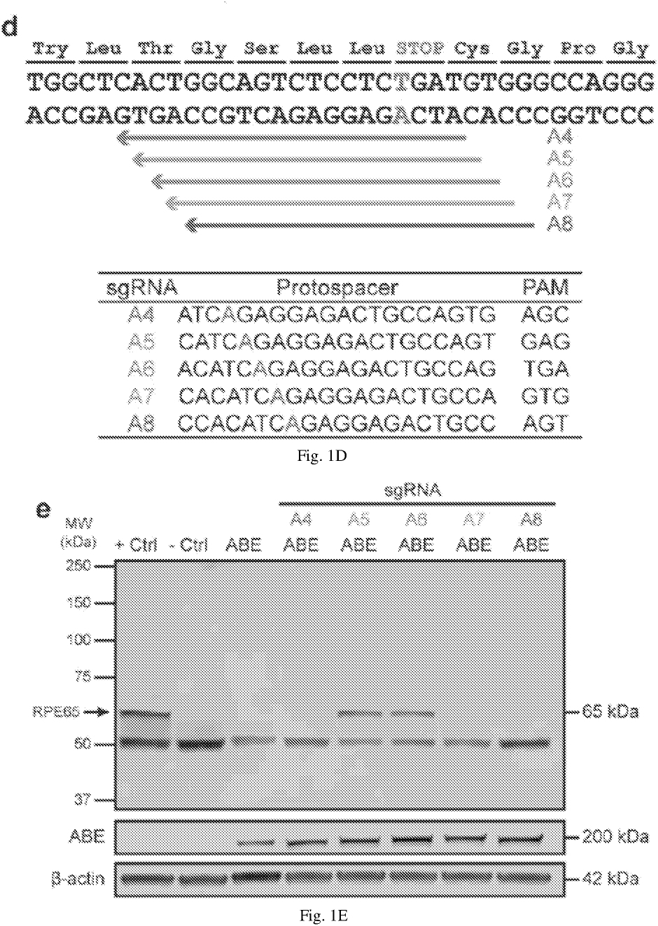

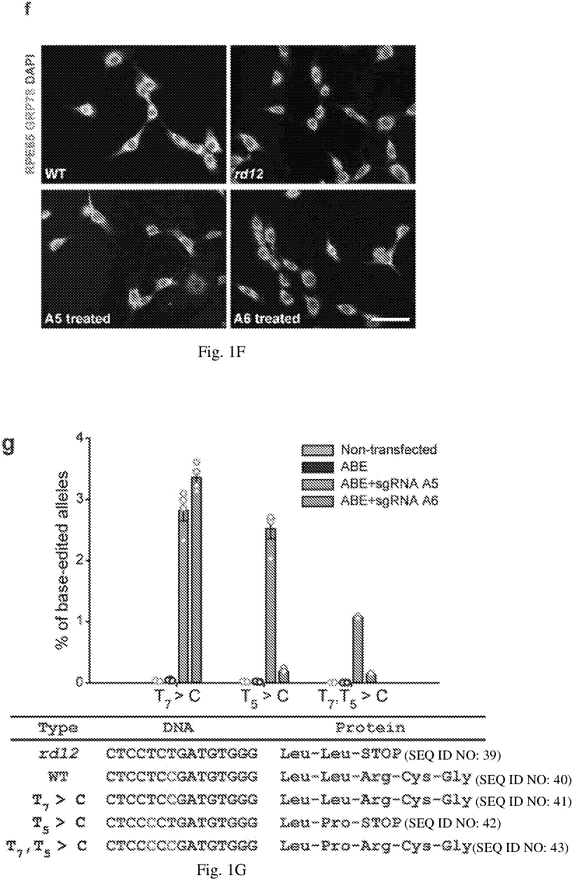

Typically, gRNAs that exist as single RNA species comprise two domains: (1) a domain that shares homology to a target nucleic acid (e.g., and directs binding of a Cas9 (or equivalent) complex to the target); and (2) a domain that binds a Cas9 protein. In some embodiments, domain (2) corresponds to a sequence known as a tracrRNA, and comprises a stem-loop structure. For example, in some embodiments, domain (2) is homologous to a tracrRNA as depicted in FIG. 1E of Jinek et al, Science 337:816-821(2012), the entire contents of which is incorporated herein by reference. Other examples of gRNAs (e.g., those including domain 2) can be found in U.S. Provisional Pat. Application, U.S.S.N. 61/874,682, filed Sep. 6, 2013, entitled “Switchable Cas9 Nucleases And Uses Thereof,” and U.S. Provisional Pat. Application, U.S.S.N. 61/874,746, filed Sep. 6, 2013, entitled “Delivery System For Functional Nucleases,” the entire contents of each are hereby incorporated by reference in their entirety. In some embodiments, a gRNA comprises two or more of domains (1) and (2), and may be referred to as an “extended gRNA.” For example, an extended gRNA will, e.g., bind two or more Cas9 proteins and bind a target nucleic acid at two or more distinct regions, as described herein. The gRNA comprises a nucleotide sequence that complements a target site, which mediates binding of the nuclease/RNA complex to said target site, providing the sequence specificity of the nuclease:RNA complex. In some embodiments, the RNA-programmable nuclease is the (CRISPR-associated system) Cas9 endonuclease, for example Cas9 (Csnl) from Streptococcus pyogenes (see, e.g., “Complete genome sequence of an Ml strain of Streptococcus pyogenes.” Ferretti J.J., McShan W.M., Ajdic D.J., Savic D.J., Savic G., Lyon K., Primeaux C., Sezate S., Suvorov A.N., Kenton S., Lai H.S., Lin S.P., Qian Y., Jia H.G., Najar F.Z., Ren Q., Zhu H., Song L., White L, Yuan X., Clifton S.W., Roe B.A., McLaughlin R.E., Proc. Natl. Acad. Sci. U.S.A. 98:4658-4663(2001);“CRISPR RNA maturation by trans-encoded small RNA and host factor RNase III.” Deltcheva E., Chylinski K., Sharma C.M., Gonzales K., Chao Y., Pirzada Z.A., Eckert M.R., Vogel L, Charpentier E., Nature 471:602-607(2011); and”A programmable dual-RNA-guided DNA endonuclease in adaptive bacterial immunity.” Jinek M., Chylinski K., Fonfara I., Hauer M., Doudna J.A., Charpentier E. Science 337:816-821(2012), the entire contents of each of which are incorporated herein by reference.

Because RNA-programmable nucleases (e.g., Cas9) use RNA:DNA hybridization to target DNA cleavage sites, these proteins are able to be targeted, in principle, to any sequence specified by the guide RNA. Methods of using RNA-programmable nucleases, such as Cas9, for site-specific cleavage (e.g., to modify a genome) are known in the art (see e.g., Cong, L. et al. Multiplex genome engineering using CRISPR/Cas systems. Science 339, 819-823 (2013); Mali, P. et al. RNA-guided human genome engineering via Cas9. Science 339, 823- 826 (2013); Hwang, W.Y. et al. Efficient genome editing in zebrafish using a CRISPR-Cas system. Nature biotechnology 31, 227-229 (2013); Jinek, M. et al. RNA-programmed genome editing in human cells. eLife 2, e00471 (2013); Dicarlo, J.E. et al. Genome engineering in Saccharomyces cerevisiae using CRISPR-Cas systems. Nucleic acids research (2013); Jiang, W. et al. RNA-guided editing of bacterial genomes using CRISPR-Cas systems. Nature biotechnology 31, 233-239 (2013); the entire contents of each of which are incorporated herein by reference).

The term “subject,” as used herein, refers to an individual organism, for example, an individual mammal. In some embodiments, the subject is a human. In some embodiments, the subject is a non-human mammal. In some embodiments, the subject is a non-human primate. In some embodiments, the subject is a rodent. In some embodiments, the subject is a sheep, a goat, a cattle, a cat, or a dog. In some embodiments, the subject is a vertebrate, an amphibian, a reptile, a fish, an insect, a fly, or a nematode. In some embodiments, the subject is a research animal. In some embodiments, the subject is genetically engineered, e.g., a genetically engineered non-human subject. The subject may be of either sex and at any stage of development. In some embodiments, the subject is an unborn subject that is in utero. In some embodiments, the subject is a zygote. In some embodiments, the subject is a blastocyst. In some embodiments, the subject is an embryo. In some embodiments, the subject is a fetus. In some embodiments, the subject has a mutation in an RPE65 gene as compared to a wild- type version. In some embodiments, the subject has a point mutation in position 130 of the RPE65 gene, which replaces a cytosine with thymine.

The term “target site” refers to a sequence within a nucleic acid molecule that is deaminated by a deaminase or a fusion protein comprising a deaminase (e.g., a Cas9-deaminase fusion protein provided herein). In some embodiments, the target site includes a mutant thymine at position 130 of exon 3 of an RPE65 gene, which can be targeted and mutated to a cytosine to correct the mutant thymine.

The terms “treatment,” “treat,” and “treating,” refer to a clinical intervention aimed to reverse, alleviate, delay the onset of, or inhibit the progress of a disease or disorder, or one or more symptoms thereof, as described herein. As used herein, the terms “treatment,” “treat,” and “treating” refer to a clinical intervention aimed to reverse, alleviate, delay the onset of, or inhibit the progress of a disease or disorder, or one or more symptoms thereof, as described herein. In some embodiments, treatment may be administered after one or more symptoms have developed and/or after a disease has been diagnosed. In other embodiments, treatment may be administered in the absence of symptoms, e.g., to prevent or delay onset of a symptom or inhibit onset or progression of a disease. For example, treatment may be administered to a susceptible individual prior to the onset of symptoms (e.g., in light of a history of symptoms and/or in light of genetic or other susceptibility factors). Treatment may also be continued after symptoms have resolved, for example, to prevent or delay their recurrence.

As used herein the term “variant” should be taken to mean the exhibition of qualities that have a pattern that deviates from what occurs in nature, e.g., a variant Cas9 is a Cas9 comprising one or more changes in amino acid residues as compared to a wild type Cas9 amino acid sequence.

As used herein the term “wild-type” is a term of the art understood by skilled persons and means the typical form of an organism, strain, gene or characteristic as it occurs in nature as distinguished from mutant or variant forms.

### DETAILED DESCRIPTION

This disclosure describes a treatment strategy for an inherited retinal disease (IRD). The strategy relies on a precise correction of a pathogenic point mutation in a mutant allele of an IRD-related gene in the retina or the retinal pigment epithelium (RPE) by subretinal delivery of a base editor (BE) system. The BE system includes a base editor and a guide RNA that targets the pathogenic mutation via viral vector or non-viral vector delivery to generate a point mutation or point mutations in the IRD-related gene. Administration of the base editor and guide RNA to a retina cell or retinal pigment epithelium can correct the pathogenic mutation, generate a non-pathogenic point mutation, or modulate (e.g., increase) expression of an IRD-related gene.

The use of a BE system, such as an adenine base editor (ABE) system, for the treatment of an IRD has unique advantages compared to prior developments of gene augmentation and CRISPR-Cas9-mediated homology-directed repair (HDR). Gene augmentation can compensate for loss-of-function RPE65 mutations by delivering a functional copy of the RPE65 gene. However, patients receiving the gene augmentation therapy continue to experience a decrease in visual sensitivity and retinal degeneration 1 to 3 years after the treatment. Although there is no clear explanation for these results, it is hypothesized that a decline in transgene expression from adeno-associated virus might be a contributing factor. Therefore, targeting the mutation with genome-editing tool can introduce permanent genomic changes.

In particular, genome editing with a BE system, such as an ABE system, can achieve a sufficient rate of precise mutation correction while minimizing undesired indel mutations and off-target effects. Base editors are comprised of either cytosine or adenosine deaminase coupled to a catalytically impaired Cas9 (dCas9), that can convert C·G to T·A base pairs or vice versa without double-stranded DNA break formation.

As described herein, base editing can provide an alternative to gene augmentation therapy to permanently rescue the function of a key vision-related protein disabled by mutations, or to correct dominant alleles for which gene augmentation may not be effective.

In comparison to the CRISPR-Cas9-mediated homology-directed repair (HDR) strategy, the base editing system provides a more accurate, precise and safer genome editing strategy. Accuracy refers to the ratio of on- versus off-target genetic changes, whereas precision relates to the fraction of on-target edits among other DNA modifications including indels. Since base editor does not induce dsDNA cleavages, there is a low likelihood of non-homologous end-joining, which is primarily responsible for indel formations.

Some aspects of this disclosure relate to methods and compositions useful for treating inherited retinal diseases (IRD), such chorioretinal atrophy or degeneration, cone or cone-rod dystrophy, congenital stationary night blindness, Leber congenital amaurosis, macular degeneration, ocular-retinal developmental disease, optic atrophy, retinitis pigmentosa, syndromic/systemic diseases with retinopathy, sorsby macular dystrophy, age-related macular degeneration, doyne honeycomb macular disease, juvenile macular degeneration, Stargardt disease, or retinitis pigmentosis. In some embodiments, the disclosure provides guide sequences capable of directing base editors (e.g., adenosine base editors) to a mutant allele of a gene to treat the IRD. In some aspects, the disclosure provides proteins that deaminate the nucleobase adenine, for example in a RPE65 gene to treat LCA.

In some embodiments, adenosine deaminase proteins are capable of deaminating (i.e., removing an amine group) adenine of a deoxy adenosine residue in deoxyribonucleic acid (DNA). For example, the adenosine deaminases provided herein are capable of deaminating adenine of a deoxy adenosine residue of DNA.

Other embodiments described herein provide fusion proteins that comprise an adenosine deaminase (e.g., an adenosine deaminase that deaminates deoxy adenosine in DNA as described herein) and a domain (e.g., a Cas9) capable of binding to a specific nucleotide sequence. The deamination of an adenosine by an adenosine deaminase can lead to a point mutation, this process is referred to herein as nucleic acid or base editing. For example, the adenosine may be converted to an inosine residue, which typically base pairs with a cytosine residue. Such fusion proteins are useful inter alia for targeted editing of nucleic acid sequences. Such fusion proteins may be used for targeted editing of DNA in vitro, e.g., for the generation of mutant cells or animals; for the introduction of targeted mutations, e.g., for the correction of genetic defects in cells ex vivo, e.g.,, in cells obtained from a subject that are subsequently re-introduced into the same or another subject; and for the introduction of targeted mutations in vivo, e.g., the correction of genetic defects or the introduction of deactivating mutations in disease-associated genes in a subject. As an example, IRDs that can be treated by making an A to G, or a T to C mutation, may be treated using the nucleobase editors described herein. The adenosine base editors described herein may be utilized for the targeted editing of such G to A mutations (e.g., targeted genome editing), for example a C130T mutation in LCA. The invention provides deaminases, fusion proteins, nucleic acids, vectors, cells, compositions, methods, kits, systems, etc. that utilize the deaminases and nucleobase editors.

In some embodiments, the nucleobase editors provided herein can be made by fusing together one or more protein domains, thereby generating a fusion protein. In certain embodiments, the fusion proteins provided herein comprise one or more features that improve the base editing activity (e.g., efficiency, selectivity, and specificity) of the fusion proteins. For example, the fusion proteins provided herein may comprise a Cas9 domain that has reduced nuclease activity. In some embodiments, the fusion proteins provided herein may have a Cas9 domain that does not have nuclease activity (dCas9), or a Cas9 domain that cuts one strand of a duplexed DNA molecule, referred to as a Cas9 nickase (nCas9).

### Base Editor

In various aspects, the instant specification provides base editors and methods of using the same to treat IRDs, such as LCA, Stargardt disease, or retinitis pigementosa. In particular, it was surprisingly found that adenosine base editors could be used to efficiently correct a C130T point mutation in the RPE65 gene both in vitro and in vivo, which is useful for the treatment of LCA with an efficiency effective to restore retinal and visual function at near normal levels.

In certain aspects, methods provided herein utilize base editors (e.g., adenosine base editors) known in the art in order to make one or more desired nucleic acid modifications. The state of the art has described numerous base editors as of this filing. The methods and approaches herein described may be applied to any previously known base editor, or to base editors that may be developed in the future. Examples of base editors that may be used in accordance with the present disclosure include those described in the following references and/or patent publications, each of which are incorporated by reference in their entireties: (a) PCT/US2014/070038 (published as WO2015/089406, Jun. 18, 2015) and its equivalents in the US or around the world; (b) PCT/US2016/058344 (published as W02017/070632, Apr. 27, 2017) and its equivalents in the US or around the world; (c) PCT/US2016/058345 (published as W02017/070633, April 27. 2017) and its equivalent in the US or around the world; (d) PCT/US2017/045381 (published as WO2018/027078, Feb. 8, 2018) and its equivalents in the US or around the world; (e) PCT/US2017/056671 (published as WO2018/071868, Apr. 19, 2018) and its equivalents in the US or around the world; PCT/2017/048390 (W02017/048390, Mar. 23, 2017) and its equivalents in the US or around the world; (f) PCT/US2017/068114 (not published) and its equivalents in the US or around the world; (g) PCT/US2017/068105 (not published)and its equivalents in the US or around the world; (h) PCT/US2017/046144 (WO2018/031683, Feb. 15, 2018) and its equivalents in the US or around the world; (i) PCT/US2018/024208 (not published) and its equivalents in the US or around the world; (j) PCT/2018/021878 (WO2018/021878, Feb. 1, 2018) and its equivalents in the US and around the world; (k) Komor, A.C., Kim, Y.B., Packer, M.S., Zuris, J.A. & Liu, D.R. Programmable editing of a target base in genomic DNA without double-stranded DNA cleavage. Nature 533, 420- (2016); (1) Gaudelli, N.M. et al. Programmable base editing of A.T to G.C in genomic DNA without DNA cleavage. Nature 551, 464- (2017); (m) any of the references listed in this specification entitled “References” and which reports or describes a base editor known in the art.

In various aspects, the base editors described herein can include a Cas moiety or napDNA/RNAbp, a nucleic acid effector domain (e.g., an adenosine deaminase), and optionally one or more nuclear localization signals (NLS). In addition, the base editors can include an optional linker that covalently joins foregoing constituents. The linkers can be any suitable type (e.g., amino acid sequences or other biopolymers, or synthetic chemical linkages in the case where the moieties are bioconjugated to one another) or length. In addition, a functional base editor would also include one or more guide sequences (e.g., guide RNA in the case of a Cas9 or Cas9 equivalent) in order to carry out the DNA/RNA-programmable functionality of base editors for targeting specific sites to be corrected.

The order of linkage of the moieties is not meant to be particularly limiting so long as the particular arrangement of the elements of moieties produces a functional base editor.

In some embodiments, the base editors provided herein can be made as a recombinant fusion protein comprising one or more protein domains, thereby generating a base editor. In certain embodiments, the base editors provided herein comprise one or more features that improve the base editing activity (e.g., efficiency, selectivity, and/or specificity) of the base editor proteins. For example, the base editor proteins provided herein may comprise a Cas9 domain that has reduced nuclease activity. In some embodiments, the base editor proteins provided herein may have a Cas9 domain that does not have nuclease activity (dCas9), or a Cas9 domain that cuts one strand of a duplexed DNA molecule, referred to as a Cas9 nickase (nCas9).

In particular, the disclosure provides adenosine base editors that can be used to correct a C130T point mutation in an RPE65 gene to treat LCA. Such adenosine base editors have been described. Exemplary domains used in base editing fusion proteins, including adenosine deaminases, napDNA/RNAbp (e.g., Cas9), and nuclear localization sequences (NLSs) are described in further detail below.

### Adenosine Deaminases

Some aspects of the disclosure provide adenosine deaminases, which are used as effector domains of base editors described herein. In some embodiments, the adenosine deaminases provided herein are capable of deaminating adenine. In some embodiments, the adenosine deaminases provided herein are capable of deaminating adenine in a deoxy adenosine residue of DNA. The adenosine deaminase may be derived from any suitable organism (e.g., E. coli). In some embodiments, the adenine deaminase is a naturally-occurring adenosine deaminase that includes one or more mutations corresponding to any of the mutations provided herein. One of skill in the art will be able to identify the corresponding residue in any homologous protein and in the respective encoding nucleic acid by methods well known in the art, e.g., by sequence alignment and determination of homologous residues. Accordingly, one of skill in the art would be able to generate mutations in any naturally-occurring adenosine deaminase that corresponds to any of the mutations described herein. In some embodiments, the adenosine deaminase is from a prokaryote. In some embodiments, the adenosine deaminase is from a bacterium. In some embodiments, the adenosine deaminase is from Escherichia coli, Staphylococcus aureus, Salmonella typhi, Shewanella putrefaciens, Haemophilus influenzae, Caulobacter crescentus, or Bacillus subtilis. In some embodiments, the adenosine deaminase is from E. coli.

In some embodiments, the adenosine deaminase is at least 50%, at least 55%, at least 60%, at least 65%, at least 70%, at least 75% at least 80%, at least 85%, at least 90%, at least 95%, at least 96%, at least 97%, at least 98%, at least 99%, or at least 99.5% identical to a naturally-occurring adenosine deaminase. In some embodiments, the adenosine deaminase is from a bacterium, such as, E.coli, S. aureus, S. typhi, S. putrefaciens, H. influenzae, or C. crescentus.

### The Cas 9 Domain of Base Editors, or Equivalent

The base editors described herein can include any suitable Cas9 moiety or equivalent protein, such as a CRISPR associated protein 9, or functional fragment thereof, and embraces any naturally-occurring Cas9 from any organism, any naturally-occurring Cas9 equivalent or functional fragment thereof, any Cas9 homolog, ortholog, or paralog from any organism, and any mutant or variant of a Cas9, naturally-occurring or engineered. More broadly, a Cas9 is a type of “RNA-programmable nuclease” or “RNA- guided nuclease” or “nucleic acid programmable DNA-binding protein.” The terms napDNA/RNAbp or Cas9 are not meant to be particularly limiting. The present disclosure is unlimited with regard to the particular napDNA/RNAbp, Cas9 or Cas9 equivalent that is employed.

In some embodiments, the napDNA/RNAbp is a Cas moiety. In various embodiments, the Cas moiety is a S. pyogenes Cas9, which has been widely used as a tool for genome engineering. This Cas9 protein is a large, multi-domain protein containing two distinct nuclease domains. Mutations, (e.g., point mutations) can be introduced into Cas9 to abolish nuclease activity of one or both of the nuclease domains, resulting in a dead Cas9 (dCas9), or a Cas9 nickase (nCas9) that still retains its ability to bind DNA in a sgRNA-programmed manner. In principle, when fused to another protein or domain, dCas9 or nCas9 can target that protein to virtually any DNA sequence simply by co expression with an appropriate sgRNA.

In other embodiments, the Cas moiety is a Cas9 from: Corynebacterium ulcerans (NCBI Refs: NC_015683.1, NC_017317.1); Corynebacterium diphtheria (NCBI Refs: NC 016782.1, NC_016786.1); Spiroplasma syrphidicola (NCBI Ref: NC_021284.1); Prevotella intermedia (NCBI Ref: NC_017861.1); Spiroplasma taiwanense (NCBI Ref: NC_021846.1); Streptococcus iniae (NCBI Ref: NC_021314.1); Belliella baltica (NCBI Ref: NC_018010.1); Psychroflexus torquisl (NCBI Ref: NC_018721.1); Streptococcus thermophilus (NCBI Ref: YP_820832.1); Listeria innocua (NCBI Ref: NP_472073.1); Campylobacter jejuni (NCBI Ref: YP_002344900.1); ox Neisseria meningitidis (NCBI Ref: YP_002342100.1 ).

In still other embodiments, the Cas moiety may include any CRISPR associated protein, including but not limited to, Casl, CaslB, Cas2, Cas3, Cas4, Cas5, Cas6, Cas7, Cas8, Cas9 (also known as Csnl and Csxl2), CaslO, xCas9, Csyl, Csy2, Csy3, Csel, Cse2, Cscl, Csc2, Csa5, Csn2. Csm2, Csm3, Csm4, Csm5, Csm6, Cmrl, Cmr3, Cmr4, Cmr5, Cmr6, Csbl, Csb2, Csb3, Csxl7, Csxl4, CsxlO, Csxl6, CsaX, Csx3, Csxl, Csxl5, Csfl, Csf2, Csf3, Csf4, homologs thereof, or modified versions thereof. These enzymes are known; for example, the amino acid sequence of S. pyogenes Cas9 protein may be found in the SwissProt database under accession number Q99ZW2.

Cas9 and equivalents can recognize a short motif in the CRISPR repeat sequences (the PAM or protospacer adjacent motif) to help distinguish self versus non-self. As noted herein, Cas9 nuclease sequences and structures are well known to those of skill in the art (see, e.g., “Complete genome sequence of an Ml strain of Streptococcus pyogenes.” Ferretti el al, J.J., McShan W.M., Ajdic D.J., Savic D.J., Savic G., Lyon K., Primeaux C., Sezate S., Suvorov A.N., Kenton S., Lai H.S., Lin S.P., Qian Y., Jia H.G., Najar F.Z., Ren Q., Zhu H., Song L., White L, Yuan X., Clifton S.W., Roe B.A., McLaughlin R.E., Proc. Natl. Acad. Sci. U.S.A. 98:4658-4663(2001);“CRISPR RNA maturation by trans-encoded small RNA and host factor RNase III.” Deltcheva E., Chylinski K., Sharma C.M., Gonzales K., Chao Y., Pirzada Z.A., Eckert M.R., Vogel L, Charpentier E., Nature 471:602-607(2011); and “A programmable dual-RNA-guided DNA endonuclease in adaptive bacterial immunity.” Jinek M., Chylinski K., Fonfara I., Hauer M., Doudna J.A., Charpentier E. Science 337:816- 821(2012), the entire contents of each of which are incorporated herein by reference).

In some embodiments, proteins comprising Cas9 or fragments thereof are referred to as “Cas9 variants.” A Cas9 variant shares homology to Cas9, or a fragment thereof. For example, a Cas9 variant is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to wild type Cas9. In some embodiments, the Cas9 variant may have 1, 2, 3, 4, 5, 6, 7, 8, 9, 10, 11, 12, 13, 14, 15, 16, 17, 18, 19, 20, 21, 22, 21, 24, 25, 26, 27, 28, 29, 30, 31, 32, 33, 34, 35, 36, 37, 38, 39, 40, 41, 42, 43, 44, 45, 46, 47, 48, 49, 50 or more amino acid changes compared to a wild type Cas9.

In some embodiments, the Cas9 variant comprises a fragment of Cas9 (e.g., a gRNA binding domain or a DNA-cleavage domain), such that the fragment is at least about 70% identical, at least about 80% identical, at least about 90% identical, at least about 95% identical, at least about 96% identical, at least about 97% identical, at least about 98% identical, at least about 99% identical, at least about 99.5% identical, or at least about 99.9% identical to the corresponding fragment of wild type Cas9. In some embodiments, the fragment is at least 30%, at least 35%, at least 40%, at least 45%, at least 50%, at least 55%, at least 60%, at least 65%, at least 70%, at least 75%, at least 80%, at least 85%, at least 90%, at least 95% identical, at least 96%, at least 97%, at least 98%, at least 99%, or at least 99.5% of the amino acid length of a corresponding wild-type Cas9.

In some embodiments, the Cas9 fragment is at least 100 amino acids in length. In some embodiments, the fragment is at least 100, 150, 200, 250, 300, 350, 400, 450, 500, 550, 600, 650, 700, 750, 800, 850, 900, 950, 1000, 1050, 1100, 1150, 1200, 1250, or at least 1300 amino acids in length. In some embodiments, wild-type Cas9 corresponds to Cas9 from Streptococcus pyogenes (NCBI Reference Sequence: NC_017053.1). In other embodiments, wild type Cas9 corresponds to Cas9 from Streptococcus pyogenes (NCBI Reference Sequence: NC_002737.2). In still other embodiments, Cas9 corresponds to, or comprises in part or in whole, a Cas9 amino acid sequence having one or more mutations that inactivate the Cas9 nuclease activity.

In some embodiments, a Cas moiety refers to a Cas9 or Cas9 homolog from archaea (e.g., nanoarchaea), which constitute a domain and kingdom of single-celled prokaryotic microbes. In some embodiments, Cas9 refers to CasX or CasY, which have been described in, for example, Burstein et al., “New CRISPR-Cas systems from uncultivated microbes.” Cell Res. 2017 Feb 21. doi: 10.1038/cr.2017.21, the entire contents of which is hereby incorporated by reference. Using genome-resolved metagenomics, a number of CRISPR-Cas systems were identified, including the first reported Cas9 in the archaeal domain of life. This divergent Cas9 protein was found in little- studied nanoarchaea as part of an active CRISPR- Cas system. In bacteria, two previously unknown systems were discovered, CRISPR-CasX and CRISPR-CasY, which are among the most compact systems yet discovered. In some embodiments, Cas9 refers to CasX, or a variant of CasX. In some embodiments, Cas9 refers to a CasY, or a variant of CasY. It should be appreciated that other RNA-guided DNA binding proteins may be used as a nucleic acid programmable DNA binding protein (napDNAbp), and are within the scope of this disclosure.

In some embodiments, the nucleic acid programmable DNA binding protein (napDNAbp) is a nucleic acid programmable DNA binding protein that does not require a canonical (NGG) PAM sequence and/or that have different PAM specificities. Typically, Cas9 proteins, such as Cas9 from S. pyogenes (spCas9), require a canonical NGG PAM sequence to bind a particular nucleic acid region. This may limit the ability to edit desired bases within a genome. In some embodiments, the base editing fusion proteins provided herein may need to be placed at a precise location, for example where a target base is placed within a 4 base region (e.g., a “deamination window”), which is approximately 15 bases upstream of the PAM. See Komor, A.C., et al, “Programmable editing of a target base in genomic DNA without double- stranded DNA cleavage” Nature 533, 420-424 (2016), the entire contents of which are hereby incorporated by reference.

Accordingly, in some embodiments, any of the fusion proteins provided herein may contain a Cas9 domain that is capable of binding a nucleotide sequence that does not contain a canonical (e.g., NGG) PAM sequence. Cas9 domains that bind to non-canonical PAM sequences have been described in the art and would be apparent to the skilled artisan. For example, Cas9 domains that bind non-canonical PAM sequences have been described in Kleinstiver, B. P., et al., “Engineered CRISPR-Cas9 nucleases with altered PAM specificities” Nature 523, 481-485 (2015); and Kleinstiver, B. P., et al., “Broadening the targeting range of Staphylococcus aureus CRISPR-Cas9 by modifying PAM recognition” Nature Biotechnology 33, 1293-1298 (2015); the entire contents of each are hereby incorporated by reference.

### Nuclear Localization Signals (NLSs)

In various embodiments, the base editors disclosed herein further comprise one or more, preferably at least two nuclear localization signals. In some embodiments, the base editors comprise at least two NLSs. In embodiments with at least two NLSs, the NLSs can be the same NLSs or they can be different NLSs. In addition, the NLSs may be expressed as part of a fusion protein with the remaining portions of the base editors. The location of the NLS fusion can be at the N-terminus, the C-terminus, or within a sequence of a base editor (e.g., inserted between the encoded napDNA/RNAbp component (e.g., Cas9) and a DNA effector moiety (e.g., a deaminase)).

The NLSs may be any known NLS sequence in the art. The NLSs may also be any future-discovered NLSs for nuclear localization. The NLSs also may be any naturally-occurring NLS, or any non-naturally occurring NLS (e.g., an NLS with one or more desired mutations).

A nuclear localization signal or sequence (NLS) is an amino acid sequence that tags, designates, or otherwise marks a protein for import into the cell nucleus by nuclear transport. Typically, this signal consists of one or more short sequences of positively charged lysines or arginines exposed on the protein surface. Different nuclear localized proteins may share the same NLS. An NLS has the opposite function of a nuclear export signal (NES), which targets proteins out of the nucleus. A nuclear localization signal can also target the exterior surface of a cell. Thus, a single nuclear localization signal can direct the entity with which it is associated to the exterior of a cell and to the nucleus of a cell. Such sequences can be of any size and composition, for example more than 25, 25, 15, 12, 10, 8, 7, 6, 5 or 4 amino acids, but will preferably comprise at least a four to eight amino acid sequence known to function as a nuclear localization signal (NLS).

The term “nuclear localization sequence” or “NLS” refers to an amino acid sequence that promotes import of a protein into the cell nucleus, for example, by nuclear transport. Nuclear localization sequences are known in the art and would be apparent to the skilled artisan. For example, NLS sequences are described in Plank et al, international PCT application, PCT/EP2000/011690, filed Nov. 23, 2000, published as WO/2001/038547 on May 31, 2001, the contents of which are incorporated herein by reference for their disclosure of exemplary nuclear localization sequences.

In some embodiments, a base editor (e.g., a known base editor, such as ABE) may be modified with one or more nuclear localization signals (NLS), preferably at least two NLSs. It will be appreciated that any nuclear localization signal known in the art at the time of the invention, or any nuclear localization signal that is identified or otherwise made available in the state of the art after the time of the instant filing can be used.

The present disclosure contemplates any suitable means by which to modify a base editor to include one or more NLSs. In one aspect, the base editors can be engineered to express a base editor protein that is translationally fused at its N-terminus or its C-terminus (or both) to one or more NLSs, i.e., to form a base editor-NLS fusion construct. In other embodiments, the base editor-encoding nucleotide sequence can be genetically modified to incorporate a reading frame that encodes one or more NLSs in an internal region of the encoded base editor. In addition, the NLSs may include various amino acid linkers or spacer regions encoded between the base editor and the N-terminally, C-terminally, or internally-attached NLS amino acid sequence, e.g., and in the central region of proteins. Thus, the present disclosure also provides for nucleotide constructs, vectors, and host cells for expressing fusion proteins that comprise a base editor and one or more NLSs.

The base editors described herein may also comprise nuclear localization signals which are linked to a base editor through one or more linkers, e.g., and polymeric, amino acid, nucleic acid, polysaccharide, chemical, or nucleic acid linker element. The linkers within the contemplated scope of the disclosure are not intended to have any limitations and can be any suitable type of molecule (e.g., polymer, amino acid, polysaccharide, nucleic acid, lipid, or any synthetic chemical linker moiety) and be joined to the base editor by any suitable strategy that effectuates forming a bond (e.g., covalent linkage, hydrogen bonding) between the base editor and the one or more NLSs.

### Linkers

In certain embodiments, linkers may be used to link any of the protein or protein domains described herein. The linker may be as simple as a covalent bond, or it may be a polymeric linker many atoms in length. In certain embodiments, the linker is a polypeptide or based on amino acids. In other embodiments, the linker is not peptide-like. In certain embodiments, the linker is a covalent bond (e.g., a carbon-carbon bond, disulfide bond, carbon-heteroatom bond, etc.). In certain embodiments, the linker is a carbon-nitrogen bond of an amide linkage. In certain embodiments, the linker is a cyclic or acyclic, substituted or unsubstituted, branched or unbranched aliphatic or hetero aliphatic linker. In certain embodiments, the linker is polymeric (e.g., polyethylene, polyethylene glycol, polyamide, polyester, etc.). In certain embodiments, the linker comprises a monomer, dimer, or polymer of aminoalkanoic acid. In certain embodiments, the linker comprises an aminoalkanoic acid (e.g., glycine, ethanoic acid, alanine, beta-alanine, 3-aminopropanoic acid, 4-aminobutanoic acid, 5-pentanoic acid, etc.). In certain embodiments, the linker comprises a monomer, dimer, or polymer of aminohexanoic acid (Ahx). In certain embodiments, the linker is based on a carbocyclic moiety (e.g., cyclopentane, cyclohexane). In other embodiments, the linker comprises a polyethylene glycol moiety (PEG). In other embodiments, the linker comprises amino acids. In certain embodiments, the linker comprises a peptide. In certain embodiments, the linker comprises an aryl or heteroaryl moiety. In certain embodiments, the linker is based on a phenyl ring. The linker may include functionalized moieties to facilitate attachment of a nucleophile (e.g., thiol, amino) from the peptide to the linker. Any electrophile may be used as part of the linker. Exemplary electrophiles include, but are not limited to, activated esters, activated amides, Michael acceptors, alkyl halides, aryl halides, acyl halides, and isothiocyanates.

In some embodiments, the linker is an amino acid or a plurality of amino acids (e.g., a peptide or protein). In some embodiments, the linker is a bond e.g., a covalent bond), an organic molecule, group, polymer, or chemical moiety.

In some embodiments, any of the fusion proteins provided herein, comprise an adenosine deaminase and a napDNAbp that are fused to each other via a linker. In some embodiments, any of the fusion proteins provided herein, comprise a first adenosine deaminase and a second adenosine deaminase that are fused to each other via a linker. In some embodiments, any of the fusion proteins provided herein, comprise an NLS, which may be fused to an adenosine deaminase (e.g., a first and/or a second adenosine deaminase), a nucleic acid programmable DNA binding protein (napDNAbp.

In some embodiments, the fusion proteins comprising an adenosine deaminase and a napDNAbp (e.g., Cas9 domain) do not include a linker sequence. In some embodiments, a linker is present between the adenosine deaminase domain and the napDNAbp. In some embodiments, the used in the general architecture above indicates the presence of an optional linker.

Some aspects of the disclosure provide fusion proteins that comprise a nucleic acid programmable DNA binding protein (napDNAbp) and at least two adenosine deaminase domains. Without wishing to be bound by any particular theory, dimerization of adenosine deaminases (e.g., in cis or in trans) may improve the ability (e.g., efficiency) of the fusion protein to modify a nucleic acid base, for example to deaminate adenine. In some embodiments, any of the fusion proteins may comprise 2, 3, 4 or 5 adenosine deaminase domains. In some embodiments, any of the fusion proteins provided herein comprise two adenosine deaminases. In some embodiments, any of the fusion proteins provided herein contain only two adenosine deaminases. In some embodiments, the adenosine deaminases are the same. In some embodiments, the adenosine deaminases are any of the adenosine deaminases provided herein. In some embodiments, the adenosine deaminases are different. In some embodiments, the first adenosine deaminase is any of the adenosine deaminases provided herein, and the second adenosine is any of the adenosine deaminases provided herein, but is not identical to the first adenosine deaminase.

It should be appreciated that the fusion proteins of the present disclosure may comprise one or more additional features. For example, in some embodiments, the fusion protein may comprise cytoplasmic localization sequences, export sequences, such as nuclear export sequences, or other localization sequences, as well as sequence tags that are useful for solubilization, purification, or detection of the fusion proteins. Suitable protein tags provided herein include, but are not limited to, biotin carboxylase carrier protein (BCCP) tags, myc-tags, calmodulin-tags, FLAG-tags, hemagglutinin (HA)-tags, polyhistidine tags, also referred to as histidine tags or His-tags, maltose binding protein (MBP)-tags, nus-tags, glutathione-S-transferase (GST)-tags, green fluorescent protein (GFP)-tags, thioredoxin-tags, S-tags, Softags (e.g.,, Softag 1, Softag 3), strep-tags , biotin ligase tags, FlAsH tags, V5 tags, and SBP-tags. Additional suitable sequences will be apparent to those of skill in the art. In some embodiments, the fusion protein comprises one or more His tags.

### Complexes of Nucleic Acid Programmable DNA Binding Proteins (napDNAbp) With Guide Nucleic Acids

Some aspects of this disclosure provide complexes comprising any of the fusion proteins (e.g., base editor) provided herein, for example any of the adenosine base editors provided herein, and a guide nucleic acid bound to napDNAbp of the fusion protein. In some embodiments, the guide nucleic acid is any one of the guide RNAs provided herein. In some embodiments, the disclosure provides any of the fusion proteins (e.g., adenosine base editors) provided herein bound to any of the guide RNAs provided herein. In some embodiments, the napDNAbp of the fusion protein (e.g., adenosine base editor) is a Cas9 domain (e.g., a Cas9, a nuclease active Cas9, or a Cas9 nickase), which is bound to a guide RNA. In some embodiments, the complexes provided herein are configured to generate a mutation in a nucleic acid, for example to correct a point mutation in a gene (e.g., RPE65) that is associated with an IRD to modulate expression of one or more proteins (e.g., RPE65) and treat the IRD, e.g., LCA.

In some embodiments, the guide RNA comprises a guide sequence that comprises at least 8, 9, 10, 11, 12, 13, 14, 15, 16, 17, 18, 19, 20, 21, 22, 23, 24, 25, 26, 27, 28, 29, or 30 contiguous nucleic acids that are 100% complementary to or hybridize with a target sequence, for example a target DNA sequence, that includes the point mutation of the IRD-related gene. In some embodiments, the guide RNA comprises a guide sequence that comprises at least 8, 9, 10, 11, 12, 13, 14, 15, 16, 17, 18, 19, 20, 21, 22, 23, 24, 25, 26, 27, 28, 29, or 30 contiguous nucleic acids that are 100% complementary to a DNA sequence in a RPE65 gene that includes the point mutation of the RPE65 gene (e.g., a target DNA sequence of any one of SEQ ID NOs: 1 or 2), for example a region of a human RPE65 gene that includes the point mutation of the IRD-related gene.

In some embodiments, any of the complexes provided herein comprise a gRNA having a guide sequence that comprises at least 8, 9, 10, 11, 12, 13, 14, 15, 16, 17, 18, 19, 20, 21, 22, 23, 24, 25, 26, 27, 28, 29, or 30 contiguous nucleic acids that are 100% complementary to any one of the nucleic acid sequences provided herein. It should be appreciated that the guide sequence of the gRNA may comprise one or more nucleotides that are not complementary to a target sequence. In some embodiments, the guide sequence of the gRNA is at the 5′ end of the gRNA. In some embodiments, the G at the 5′ end of the gRNA is not complementary with the target sequence. In some embodiments, the guide sequence of the gRNA comprises 1, 2, 3, 4, 5, 6, 7, or 8 nucleotides that are not complementary to a target sequence.

In some embodiments, the guide RNA comprises a guide sequence that comprises at least 8, 9, 10, 11, 12, 13, 14, 15, 16, 17, 18, 19, 20, 21, 22, 23, 24, 25, 26, 27, 28, 29, or 30 contiguous nucleic acids that are 100% complementary to a target sequence that includes the point mutation of the IRD-related gene, for example a target DNA sequence in a RPE65 gene. In some embodiments, the guide RNA comprises a guide sequence that comprises at least 8, 9, 10, 11, 12, 13, 14, 15, 16, 17, 18, 19, 20, 21, 22, 23, 24, 25, 26, 27, 28, 29, or 30 contiguous nucleic acids that are 100% complementary to a DNA sequence in a human RPE65 gene. In some embodiments, the RPE gene is a human, chimpanzee, ape, monkey, dog, mouse, or rat RPE65 gene. In some embodiments, the RPE65 gene is a human RPE65 gene.

In some embodiments, the guide sequences are capable of guiding a base editor to correct a mutation in RPE65 (e.g., a C130T point mutation in RPE65). In various embodiments base editors (e.g., base editors provided herein) can be complexed, bound, or otherwise associated with (e.g., via any type of covalent or non-covalent bond) one or more guide sequences, i.e., the sequence which becomes associated or bound to the base editor and directs its localization to a specific target sequence having complementarity to the guide sequence or a portion thereof. The particular design aspects of a guide sequence will depend upon the nucleotide sequence of a genomic target site of interest (e.g., the mutant T130 residue of human RPE65) and the type of napDNA/RNAbp (e.g., type of Cas protein) present in the base editor, among other factors, such as PAM sequence locations, percent G/C content in the target sequence, the degree of microhomology regions, secondary structures, etc.

In general, a guide sequence can include any polynucleotide sequence having sufficient complementarity with a target polynucleotide sequence to hybridize with the target sequence and direct sequence-specific binding of a napDNARNAbp (e.g., a Cas9, Cas9 homolog, or Cas9 variant) to the target sequence, such as a sequence within an RPE65 gene that comprises a C130T point mutation. In some embodiments, the degree of complementarity between a guide sequence and its corresponding target sequence (e.g., RPE65), when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith- Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies, ELAND (Illumina, San Diego, Calif.), SOAP (available at soap.genomics.org.cn), and Maq (available at maq.sourceforge.net).

In some embodiments, a guide sequence is about or more than about 5, 10, 11, 12, 13, 14, 15, 16, 17, 18, 19, 20, 21, 22, 23, 24, 25, 26, 27, 28, 29, 30, 31, 32, 33, 34, 35, 40, 45, 50, 75, or more nucleotides in length.

In some embodiments, a guide sequence is less than about 75, 50, 45, 40, 35, 30, 25, 20, 15, 12, or fewer nucleotides in length.

The ability of a guide sequence to direct sequence- specific binding of a base editor to a target sequence may be assessed by any suitable assay. For example, the components of a base editor, including the guide sequence to be tested, may be provided to a host cell having the corresponding target sequence (e.g., a NIH3T3 cell line), such as by transfection with vectors encoding the components of a base editor disclosed herein, followed by an assessment of preferential cleavage within the target sequence. Similarly, cleavage of a target polynucleotide sequence may be evaluated in a test tube by providing the target sequence, components of a base editor, including the guide sequence to be tested and a control guide sequence different from the test guide sequence, and comparing binding or rate of cleavage at the target sequence between the test and control guide sequence reactions. Other assays are possible, and will occur to those skilled in the art.

In some embodiments, a guide sequence is provided that is designed to target a C130T point mutation in RPE65. In some embodiments, the target sequence is a RPE65 sequence within a genome of a cell. An exemplary sequence within the human RPE65 gene that contains a C130T point mutation is provided below. It should be appreciated, however that additional exemplary RPE65 gene sequences are within the scope of this disclosure and guide RNAs can be designed to accommodate any differences between RPE65 sequences provided herein and any RPE65 sequences, or variants thereof (e.g., mutants), found in nature.

In some embodiments, portions of a mouse RPE65 gene and homo sapien RPE65 gene, on exon 3, that include the C130T residue, which when mutated, leads to the development of LCA, can have, respectively, the following nucleotide sequences. The C130T residue is indicated in bold.

5′-CTCACTG5GCAGTCTCCTCTGATGTGGGCCA -3′
(SEQ ID NO: 1)

5′-CTCACCGGCAGTCTCCTCTGATGTGGGCCA -3′
(SEQ ID NO:2)

Additional examples of portions of the RPE65 gene that include the C130T point mutation can have the following nucleotide sequences:

5′-TCACTGGCAGTCTCCTCTGATGTGGGCCAG-3′
(SEQ ID NO: 3)

5′-CACTGGCAGTCTCCTCTGATGTGGGCCAGG-3′
(SEQ ID NO: 4)

5′-ACTGGCAGTCTCCTCTGATGTGGGCCAGGG-3′
(SEQ ID NO: 5)

5′-GCTCACTGGCAGTCTCCTCTGATGTGGGCC-3′
(SEQ ID NO: 6)

5′-GGCTCACTGGCAGTCTCCTCTGATGTGGGC-3′
(SEQ ID NO: 7)

5′-TGGCTCACTGGCAGTCTCCTCTGATGTGGG-3′
(SEQ ID NO: 8)

5′-TCACCGGCAGTCTCCTTTGATGTGGGCCAG-3′
(SEQ ID NO: 9)

5′-CACCGGCAGTCTCCTTTGATGTGGGCCAGG-3′
(SEQ ID NO: 10)

5′-ACCGGCAGTCTCCTTTGATGTGGGCCAGGG-3′
(SEQ ID NO: 11)

5′-GCTCACCGGCAGTCTCCTTTGATGTGGGCC-3′
(SEQ ID NO: 12)

5′-GGCTCACCGGCAGTCTCCTTTGATGTGGGC-3′
(SEQ ID NO: 13)

5′-TGGCTCACCGGCAGTCTCCTTTGATGTGGGC-3′
(SEQ ID NO: 14)

The disclosure also contemplates portions of the RPE65 gene that are shorter or longer than any one of the exemplary portions of the RPE65 gene provided in any one of SEQ ID NOs: 1-14. It should be appreciated that guide sequences may be engineered that are complementary (e.g., 100% complementary) to any of the exemplary portions of the RPE65 gene provided herein (e.g., SEQ ID NOs: 1-14).

In some embodiments, a guide sequence is complementary (e.g., 100% complementary) to any one of SEQ ID NOs: 1-14. In some embodiments, a guide sequence is complementary (e.g., 100% complementary) to a sequence of any one of SEQ ID NOs: 1-14 absent the first 1, 2, 3, 4, 5, 7, or 8 nucleic acid residues at the 5′ end.

In some embodiments, a guide sequence is complementary (e.g., 100% complementary) to a sequence of any one of SEQ ID NOs: 1-4 absent the first 1, 2, 3, 4, 5, 7, or 8 nucleic acid residues at the 3′ end.

The guide sequence is typically about 20 nucleotides long. Exemplary guide sequences for targeting a base editor (e.g., cABE) to a site comprising a C130T point mutation in RPE65 are provided below. It should be appreciated, however, that changes to such guide sequences can be made based on the specific RPE65 sequence found within a cell, for example a cell of a patient having LCA.

Such suitable guide RNA sequences typically comprise guide sequences that are complementary to a target nucleic sequence within 50 nucleotides upstream or downstream of the target nucleotide to be edited.

In some embodiments, the nucleic acid sequence of DNA encoding the guide sequence can include at least one of:

5′-ATCAGAGGAGACTGCCAGTG-3 ′ (SEQ ID NO: 15),

5′-CATCAGAGGAGACTGCCAGT-3 ′ (SEQ ID NO: 16),

5′-ACATCAGAGGAGACTGCCAG-3 ′ (SEQ ID NO: 17),

5′-CACATCAGAGGAGACTGCCA-3 ′ (SEQ ID NO: 18),

5′-CCACATCAGAGGAGACTGCC-3 ′ (SEQ ID NO: 19),

5′-ATCAAAGGAGACTGCCGGTG-3 ′ (SEQ ID NO: 20),

5′-CATCAAAGGAGACTGCCGGT-3 ′ (SEQ ID NO: 21),

5′-ACATCAAAGGAGACTGCCGG-3 ′ (SEQ ID NO: 22),

5′-CACATCAAAGGAGACTGCCG-3 ′ (SEQ ID NO: 23), or

5′-CCACATCAAAGGAGACTGCC-3 ′ (SEQ ID NO: 24).

Examples of guide sequences that can target a C130T point mutation in RPE65 of a mouse or human include the following:

5′-AUCAGAGGAGACUGCCAGUG-3 ′ (SEQ ID NO: 25),

5′-CAUCAGAGGAGACUGCCAGU-3 ′ (SEQ ID NO: 26),

5′-ACAUCAGAGGAGACUGCCAG-3 ′ (SEQ ID NO: 27),

5′-CACAUCAGAGGAGACUGCCA-3 ′ (SEQ ID NO: 28),

5′-CCACAUCAGAGGAGACUGCC-3 ′ (SEQ ID NO: 29),

5′-AUCAAAGGAGACUGCCGGUG-3 ′ (SEQ ID NO: 30),

5′-CAUCAAAGGAGACUGCCGGU-3 ′ (SEQ ID NO: 31),

5′-ACAUCAAAGGAGACUGCCGG-3 ′ (SEQ ID NO: 32),

5′-CACAUCAAAGGAGACUGCCG-3 ′ (SEQ ID NO: 33), or

5′-CCACAUCAAAGGAGACUGCC-3 ′ (SEQ ID NO: 34).

In some embodiments, a guide sequence is selected to reduce the degree of secondary structure within the guide sequence. Secondary structure may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online Webserver RNAfold, developed at Institute for Theoretical Chemistry at the University of Vienna, using the centroid structure prediction algorithm (see e.g., A. R. Gruber et al., 2008, Cell 106(1): 23-24; and PA Carr and GM Church, 2009, Nature Biotechnology 27(12): 1151- 62). Further algorithms may be found in U.S. Application Ser. No. 61/836,080; Broad Reference BI-2013/004A); incorporated herein by reference.

The disclosure also provides guide sequences that are truncated variants of any of the guide sequences provided herein (e.g., SEQ ID NOs: 25-34). The disclosure also provides guide sequences that are longer variants of any of the guide sequences provided herein (e.g., SEQ ID NOs: 25-34). In some embodiments, the guide sequence comprises one, two, three, four, five, or 6 additional residue that is at the 5′ or at the 3′ end of any one of SEQ ID NOs: 25-34.

Some aspects of this disclosure provide methods of using the fusion proteins, or complexes comprising a guide nucleic acid (e.g., gRNA) and a nucleobase editor provided herein. For example, some aspects of this disclosure provide methods comprising contacting a DNA, or RNA molecule with any of the fusion proteins provided herein, and with at least one guide nucleic acid (e.g., guide RNA), wherein the guide nucleic acid, (e.g., guide RNA) comprises a sequence (e.g., a guide sequence that binds to a DNA target sequence) of at least 10 (e.g., at least 10, 15, 20, 25, or 30) contiguous nucleotides that is 100% complementary to a target sequence (e.g., any of the target RPE65 sequences provided herein).

In some embodiments, the 3′ end of the target sequence is immediately adjacent to a canonical PAM sequence (NGG). In some embodiments, the 3′ end of the target sequence is not immediately adjacent to a canonical PAM sequence (NGG). In some embodiments, the 3′ end of the target sequence is immediately adjacent to a non-canonical PAM sequence, such as AGC, GAG, TGA, GTG, or AGT sequence. It was found that wt-optimized codon ABE yields a stable and higher protein expression, leading to more frequent base editing activity even at sites lacking a canonical NGG PAM.

### Correcting Mutations in an IRD Related Gene

Some aspects of the disclosure provide methods of using base editors (e.g., any of the fusion proteins provided herein) and gRNAs to correct a point mutation (e.g., a C130T mutation) in an IRD related gene (e.g., RPE65 gene). Exemplary portions of a human RPE65 gene comprising a T at position 130 (indicated in bold) are provided in SEQ ID NOs: 1-14. In some embodiments, the disclosure provides methods of using base editors (e.g., any of the fusion proteins provided herein) and gRNAs to generate an A to G and/or T to C mutation in an RPE65 gene. In some embodiments, the disclosure provides methods for deaminating an adenosine nucleobase (A) in an RPE65 gene, the method comprising contacting the RPE65 gene with a base editor and a guide RNA bound to the base editor, where the guide RNA comprises a guide sequence that is complementary to a target nucleic acid sequence in the RPE65 gene. In some embodiments, the RPE65 gene comprises a C to T or G to A mutation. In some embodiments, the C to T or G to A mutation in the RPE65 gene impairs function of the RPE65 protein encoded by the RPE gene. In some embodiments, the C to T or G to A mutation in the RPE65 gene is nonsense mutation that results in a decrease in expression of at least 1%, 2%, 5%, 10%, 15%, 20%, 25%, 30%, 35%, 40%, 45%, 50%, 55%, 60%, 65%, 70%, 75%, 80%, 85%, 90%, 95%, 98%, or at least 99% RPE65.

In some embodiments, deaminating an adenosine (A) nucleobase complementary to the T corrects the C to T or G to A mutation in the RPE65 gene. In some embodiments, the C to T or G to A mutation in the RPE65 gene leads to a Cys (C) to Tyr (Y) mutation in the RPE65 protein encoded by the RPE65 gene. In some embodiments, deaminating the adenosine nucleobase complementary to the T corrects the Cys to Tyr mutation in the RPE65 protein.

In some embodiments, the guide sequence of the gRNA comprises at least 8, 9, 10, 11, 12, 13, 14, 15, 16, 17, 18, 19, 20, 21, 22, 23, 24, 25, 26, 27, 28, 29, 30, 31, 32, 33, 34, or 35 contiguous nucleic acids that are 100% complementary to a target nucleic acid sequence of the RPE65 gene. In some embodiments, the base editor nicks the target sequence that is complementary to the guide sequence.

In some embodiments, the target DNA sequence comprises a sequence associated with an IRD or disorder, e.g., LCA. In some embodiments, the target DNA sequence comprises a point mutation associated with a disease or disorder. In some embodiments, the activity of the fusion protein (e.g., comprising an adenosine deaminase and a Cas9 domain), or the complex, results in a correction of the point mutation. In some embodiments, the target DNA sequence comprises a G to A or C to T point mutation associated with a IRD, and wherein the deamination of the mutant base results in a sequence that is not associated with a disease or disorder. In some embodiments, the target DNA sequence encodes a protein, and the point mutation is in a codon and results in a change in the amino acid encoded by the mutant codon as compared to the wild-type codon. In some embodiments, the deamination of the mutant base results in a change of the amino acid encoded by the mutant codon. In some embodiments, the deamination of the mutant base results in the codon encoding the wild-type amino acid. In some embodiments, the contacting is in vivo in a subject. In some embodiments, the subject has or has been diagnosed with an IRD.

In some embodiments, the purpose of the methods provided herein is to restore the function of a dysfunctional gene via genome editing. The nucleobase editing proteins provided herein can be validated for gene editing-based human therapeutics in vitro, e.g., by correcting an IRD-associated mutation in human cell culture. It will be understood by the skilled artisan that the nucleobase editing proteins provided herein, e.g., the fusion proteins comprising a nucleic acid programmable DNA binding protein (e.g., Cas9) and an adenosine deaminase domain can be used to correct any single point G to A or C to T mutation. In the first case, deamination of the mutant A to G corrects the mutation, and in the latter case, deamination of the A that is base-paired with the mutant T, followed by a round of replication or followed by base editing repair activity, corrects the mutation.

The instant disclosure provides methods for the treatment of a subject diagnosed with an IRD associated with or caused by a point mutation that can be corrected by a DNA editing fusion protein provided herein.

In some embodiments, a fusion protein recognizes canonical or noncanonical PAMs and therefore can correct the pathogenic G to A or C to T mutations with canonical or non-canonical PAMs, e.g., NG, NGG, AGC, GAG, TGA, GTG, or AGT, respectively, in the flanking sequences.

It will be apparent to those of skill in the art that in order to target any of the fusion proteins comprising a Cas9 domain and an adenosine deaminase, as disclosed herein, to a target site, e.g., a site comprising a point mutation to be edited, it is typically necessary to co-express the fusion protein together with a guide RNA, e.g., an sgRNA.

### Methods for Editing Nucleic Acids

Some aspects of the disclosure provide methods for editing a nucleic acid. In some embodiments, the method is a method for editing a nucleobase of a nucleic acid (e.g., a base pair of a double-stranded DNA sequence). In some embodiments, the method comprises the steps of: a) contacting a target region of a nucleic acid (e.g., a double-stranded DNA sequence) with a complex comprising a base editor (e.g., a Cas9 domain fused to an adenosine deaminase) and a guide nucleic acid (e.g., gRNA), wherein the target region comprises a targeted nucleobase pair, b) inducing strand separation of said target region, c) converting a first nucleobase of said target nucleobase pair in a single strand of the target region to a second nucleobase, and d) cutting no more than one strand of said target region, where a third nucleobase complementary to the first nucleobase base is replaced by a fourth nucleobase complementary to the second nucleobase. In some embodiments, the method results in less than 20%, 19%, 18%, 17%, 16%, 15%, 14%, 13%, 12%, 11%, 10%, 9%, 8%, 7%, 6%, 5%, 4%, 3%, 2%, 1% or indel formation in the nucleic acid. It should be appreciated that in some embodiments, step b is omitted. In some embodiments, the first nucleobase is an adenine. In some embodiments, the second nucleobase is a deaminated adenine, or inosine.

In some embodiments, the third nucleobase is a thymine. In some embodiments, the fourth nucleobase is a cytosine. In some embodiments, the method results in less than 19%, 18%, 16%, 14%, 12%, 10%, 8%, 6%, 4%, 2%, 1%, 0.5%, 0.2%, or less than 0.1% indel formation. In some embodiments, the method further comprises replacing the second nucleobase with a fifth nucleobase that is complementary to the fourth nucleobase, thereby generating an intended edited base pair (e.g., A:T to G:C). In some embodiments, the fifth nucleobase is a guanine. In some embodiments, at least 5% of the intended base pairs are edited. In some embodiments, at least 10%, 15%, 20%, 25%, 30%, 35%, 40%, 45%, or 50% of the intended base pairs are edited.

In some embodiments, the intended edited base pair is 1, 2, 3, 4, 5, 6, 7, 8, 9, 10, 11, 12, 13, 14, 15, 16, 17, 18, 19, or 20 nucleotides downstream stream of the PAM site. In some embodiments, the method does not require a canonical (e.g., NGG) PAM site.

In some embodiments, the disclosure provides methods for editing a nucleotide. In some embodiments, the disclosure provides a method for editing a nucleobase pair of a double-stranded DNA sequence. In some embodiments, the method comprises a) contacting a target region of the double-stranded DNA sequence with a complex comprising a base editor and a guide nucleic acid (e.g., gRNA), where the target region comprises a target nucleobase pair, b) inducing strand separation of said target region, c) converting a first nucleobase of said target nucleobase pair in a single strand of the target region to a second nucleobase, d) cutting no more than one strand of said target region, wherein a third nucleobase complementary to the first nucleobase base is replaced by a fourth nucleobase complementary to the second nucleobase, and the second nucleobase is replaced with a fifth nucleobase that is complementary to the fourth nucleobase, thereby generating an intended edited base pair, wherein the efficiency of generating the intended edited base pair is at least 5%. It should be appreciated that in some embodiments, step b is omitted. In some embodiments, at least 5% of the intended base pairs are edited. In some embodiments, at least 10%, 15%, 20%, 25%, 30%, 35%, 40%, 45%, or 50% of the intended base pairs are edited. In some embodiments, the method causes less than 19%, 18%, 16%, 14%, 12%, 10%, 8%, 6%, 4%, 2%, 1%, 0.5%, 0.2%, or less than 0.1% indel formation.

### Expression Vectors

The base editors (and their associated gRNAs) may be expressed in a cell of interest by incorporating a nucleic acid encoding base editors (and their associated gRNAs) interest into an appropriate expression vector. As used herein, “expression vector” refers to a vector comprising a recombinant polynucleotide comprising expression control sequences operatively linked to a nucleotide sequence to be expressed. An expression vector comprises sufficient cis- acting elements for expression; other elements for expression can be supplied by the host cell or in an in vitro expression system. Expression vectors include all those known in the art, such as cosmids, plasmids (e.g., naked or contained in liposomes), retrotransposons (e.g., piggyback, sleeping beauty), and viruses (e.g., lentiviruses, retroviruses, adenoviruses, and adeno-associated viruses) that incorporate the recombinant polynucleotide of interest.

In certain embodiments, the expression vector is a viral vector. The term “virus” is used herein to refer to an obligate intracellular parasite having no protein-synthesizing or energy-generating mechanism. Exemplary viral vectors include retroviral vectors (e.g., lentiviral vectors), adenoviral vectors, adeno-associated viral vectors, herpesviruses vectors, epstein-barr virus (EBV) vectors, polyomavirus vectors (e.g., simian vacuolating virus 40 (SV40) vectors), poxvirus vectors, and pseudotype virus vectors.

The virus may be an RNA virus (having a genome that is composed of RNA) or a DNA virus (having a genome composed of DNA). In certain embodiments, the viral vector is a DNA virus vector. Examples of DNA viruses include parvoviruses (e.g., adeno-associated viruses), adenoviruses, asfarviruses, herpesviruses (e.g., herpes simplex virus 1 and 2 (HSV-1 and HSV-2), epstein-barr virus (EBV), cytomegalovirus (CMV)), papillomoviruses (e.g., HPV), polyomaviruses (e.g., simian vacuolating virus 40 (SV40)), and poxviruses (e.g., vaccinia virus, cowpox virus, smallpox virus, fowlpox virus, sheeppox virus, myxoma virus). In certain embodiments, the viral vector is an RNA virus vector. Examples of RNA viruses include bunyaviruses (e.g., hantavirus), coronaviruses, flaviviruses (e.g., yellow fever virus, west nile virus, dengue virus), hepatitis viruses (e.g., hepatitis A virus, hepatitis C virus, hepatitis E virus), influenza viruses (e.g., influenza virus type A, influenza virus type B, influenza virus type C), measles virus, mumps virus, noroviruses (e.g., Norwalk virus), poliovirus, respiratory syncytial virus (RSV), retroviruses (e.g., human immunodeficiency virus-1 (HIV-1)) and toroviruses.

In certain embodiments, the expression vector comprises a regulatory sequence or promoter operably linked to the nucleotide sequence encoding the tRNA. The term “operably linked” refers to a linkage of polynucleotide elements in a functional relationship. A nucleic acid sequence is “operably linked” when it is placed into a functional relationship with another nucleic acid sequence. For instance, a promoter or enhancer is operably linked to a gene if it affects the transcription of the gene. Operably linked nucleotide sequences are typically contiguous. However, as enhancers generally function when separated from the promoter by several kilobases and intronic sequences may be of variable lengths, some polynucleotide elements may be operably linked but not directly flanked and may even function in trans from a different allele or chromosome.

### Adeno-Associated Virus (AAV) Vectors

In certain embodiments, an expression vector is an adeno-associated virus (AAV) vector. AAV is a small, nonenveloped icosahedral virus of the genus Dependoparvovirus and family Parvovirus. AAV has a single-stranded linear DNA genome of approximately 4.7 kb. AAV is capable of infecting both dividing and quiescent cells of several tissue types, with different AAV serotypes exhibiting different tissue tropism.

AAV includes numerous serologically distinguishable types including serotypes AAV-1 to AAV-12, as well as more than 100 serotypes from nonhuman primates (See, e.g., Srivastava (2008) J. CELL BIOCHEM., 105(1): 17-24, and Gao et al. (2004) J. VIROL., 78(12), 6381-6388). The serotype of the AAV vector used in the methods and compositions described herein can be selected by a skilled person in the art based on the efficiency of delivery, tissue tropism, and immunogenicity. AAV serotypes identified from rhesus monkeys, e.g., rh.8, rh.10, rh.39, rh.43, and rh.74, are also contemplated in the compositions and methods described herein. Besides the natural AAV serotypes, modified AAV capsids have been developed for improving efficiency of delivery, tissue tropism, and immunogenicity. Exemplary natural and modified AAV capsids are disclosed in U.S. Pat. Nos. 7,906,111, 9,493,788, and 7,198,951, and PCT Publication No. WO2017189964A2.

The wild-type AAV genome contains two 145 nucleotide inverted terminal repeats (ITRs), which contain signal sequences directing AAV replication, genome encapsidation and integration. In addition to the ITRs, three AAV promoters, p5, p19, and p40, drive expression of two open reading frames encoding rep and cap genes. Two rep promoters, coupled with differential splicing of the single AAV intron, result in the production of four rep proteins (Rep 78, Rep 68, Rep 52, and Rep 40) from the rep gene. Rep proteins are responsible for genomic replication. The Cap gene is expressed from the p40 promoter, and encodes three capsid proteins (VP1, VP2, and VP3) which are splice variants of the cap gene. These proteins form the capsid of the AAV particle.

Because the cis-acting signals for replication, encapsidation, and integration are contained within the ITRs, some or all of the 4.3 kb internal genome may be replaced with foreign DNA, for example, an expression cassette for an exogenous gene of interest. Accordingly, in certain embodiments, the AAV vector comprises a genome comprising an expression cassette for an exogenous gene flanked by a 5′ ITR and a 3′ ITR. The ITRs may be derived from the same serotype as the capsid or a derivative thereof. Alternatively, the ITRs may be of a different serotype from the capsid, thereby generating a pseudotyped AAV. In certain embodiments, the ITRs are derived from AAV-2. In certain embodiments, the ITRs are derived from AAV-5. At least one of the ITRs may be modified to mutate or delete the terminal resolution site, thereby allowing production of a self-complementary AAV vector.

The rep and cap proteins can be provided in trans, for example, on a plasmid, to produce an AAV vector. A host cell line permissive of AAV replication must express the rep and cap genes, the ITR-flanked expression cassette, and helper functions provided by a helper virus, for example adenoviral genes E1a, E1b55K, E2a, E4orf6, and VA (Weitzman et al., Adeno-associated virus biology. Adeno-Associated Virus: Methods and Protocols, pp. 1-23, 2011). Methods for generating and purifying AAV vectors have been described in detail (See e.g., Mueller et al., (2012) CURRENT PROTOCOLS IN MICROBIOLOGY, 14D.1.1-14D.1.21, Production and Discovery of Novel Recombinant Adeno-Associated Viral Vectors). Numerous cell types can be used for producing AAV vectors, including HEK293 cells, COS cells, HeLa cells, BHK cells, Vero cells, as well as insect cells (See e.g., U.S. Pat. Nos. 6,156,303, 5,387,484, 5,741,683, 5,691,176, 5,688,676, and 8,163,543, U.S. Pat. Publication No. 20020081721, and PCT Publication Nos. WO00/47757, WO00/24916, and WO96/17947). AAV vectors are typically produced in these cell types by one plasmid containing the ITR-flanked expression cassette, and one or more additional plasmids providing the additional AAV and helper virus genes.

AAV of any serotype may be used in the methods and compositions described herein. Similarly, it is contemplated that any adenoviral type may be used, and a person of skill in the art will be able to identify AAV and adenoviral types that can be used for the production of their desired recombinant AAV vector (rAAV). AAV particles may be purified, for example, by affinity chromatography, iodixonal gradient, or CsCl gradient.

AAV vectors may have single-stranded genomes that are 4.7 kb in size, or are larger or smaller than 4.7 kb, including oversized genomes that are as large as 5.2 kb, or as small as 3.0 kb. Thus, where the exogenous gene of interest to be expressed from the AAV vector is small, the AAV genome may comprise a stuffer sequence. Further, vector genomes may be substantially self-complementary thereby allowing for rapid expression in the cell. In certain embodiments, the genome of a self-complementary AAV vector comprises from 5′ to 3′: a 5′ ITR; a first nucleic acid sequence comprising a promoter and/or enhancer operably linked to a coding sequence of a gene of interest; a modified ITR that does not have a functional terminal resolution site; a second nucleic acid sequence complementary or substantially complementary to the first nucleic acid sequence; and a 3′ ITR. AAV vectors containing genomes of all types are can be used in the methods described herein.

Non-limiting examples of AAV vectors include pAAV-MCS (Agilent Technologies), pAAVK-EF1α-MCS (System Bio Catalog # AAV502A-1), pAAVK-EF1α-MCS1-CMV-MCS2 (System Bio Catalog # AAV503A-1), pAAV-ZsGreen1 (Clontech Catalog #6231), pAAV-MCS2 (Addgene Plasmid #46954), AAV-Stuffer (Addgene Plasmid #106248), pAAVscCBPIGpluc (Addgene Plasmid #35645), AAVS1_Puro_PGK1_3xFLAG_Twin_Strep (Addgene Plasmid #68375), pAAV-RAM-d2TTA::TRE-MCS-WPRE-pA (Addgene Plasmid #63931), pAAV-UbC (Addgene Plasmid #62806), pAAVS1-P-MCS (Addgene Plasmid #80488), pAAV-Gateway (Addgene Plasmid #32671), pAAV-Puro_siKD (Addgene Plasmid #86695), pAAVS1-Nst-MCS (Addgene Plasmid #80487), pAAVS1-Nst-CAG-DEST (Addgene Plasmid #80489), pAAVS1-P-CAG-DEST (Addgene Plasmid #80490), pAAVf-EnhCB-lacZnls (Addgene Plasmid #35642), and pAAVS1-shRNA (Addgene Plasmid #82697). These vectors can be modified for therapeutic use. For example, an exogenous gene of interest can be inserted in a multiple cloning site, and a selection marker (e.g., puro or a gene encoding a fluorescent protein) can be deleted or replaced with another (same or different) exogenous gene of interest. Further examples of AAV vectors are disclosed in U.S. Pat. Nos. 5,871,982, 6,270,996, 7,238,526, 6,943,019, 6,953,690, 9,150,882, and 8,298,818, U.S. Pat. Publication No. 2009/0087413, and PCT Publication Nos. WO2017075335A1, WO2017075338A2, and WO2017201258A1.

In certain embodiments, delivery of the base editor and sgRNA uses a split-base editor dual AAV strategy. One impediment to the delivery of base editors in animals has been an inability to package base editors in adeno-associated virus (AAV), an efficient and widely used delivery agent that remains the only FDA-approved in vivo gene therapy vector. The large size of the DNA encoding base editors (5.2 kb for base editors containing S. pyogenes Cas9, not including any guide RNA or regulatory sequences) can preclude packaging in AAV, which has a genome packaging size limit of <5 kb 12.

To bypass this packaging size limit and deliver base editors using AAVs, a split-base editor dual AAV strategy can be used, in which the adenine base editor (ABE) is divided into an N-terminal and C- terminal half. This strategy is described in U.S. Provisional Pat. Application U.S.S.N. 62/850,523, filed on May 20, 2019; the entire contents of which are hereby incorporated by reference. Each base editor half is fused to half of a fast-splicing split-intein. Following co-infection by AAV particles expressing each base editor-split intein half, protein splicing in trans reconstitutes full-length base editor. Unlike other approaches utilizing small molecules or sgRNA to bridge split Cas9, intein splicing removes all exogenous sequences and regenerates a native peptide bond at the split site, resulting in a single reconstituted protein identical in sequence to the unmodified base editor.

Split-intein ABEs were developed and integrated into optimized dual AAV genomes to enable efficient base editing in somatic tissues of therapeutic relevance, including retina. The resulting AAVs were used to achieve base editing efficiencies at test loci for ABEs as well as cytosine base editors (CBEs) that, in each of these tissues, meets or exceeds therapeutically relevant editing thresholds for the treatment of some human genetic diseases at AAV dosages that are known to be well-tolerated in humans.

### Lentivirus Vectors

In certain embodiments, the viral vector can be a retroviral vector. Examples of retroviral vectors include moloney murine leukemia virus vectors, spleen necrosis virus vectors, and vectors derived from retroviruses, such as rous sarcoma virus, harvey sarcoma virus, avian leukosis virus, human immunodeficiency virus, myeloproliferative sarcoma virus, and mammary tumor virus. Retroviral vectors are useful as agents to mediate retroviral-mediated gene transfer into eukaryotic cells.

In certain embodiments, the retroviral vector is a lentiviral vector. Exemplary lentiviral vectors include vectors derived from human immunodeficiency virus-1 (HIV-1), human immunodeficiency virus-2 (HIV-2), simian immunodeficiency virus (SIV), feline immunodeficiency virus (FIV), bovine immunodeficiency virus (BIV), Jembrana Disease Virus (JDV), equine infectious anemia virus (EIAV), and caprine arthritis encephalitis virus (CAEV).

Retroviral vectors typically are constructed such that the majority of sequences coding for the structural genes of the virus are deleted and replaced by the gene(s) of interest. Often, the structural genes (i.e., gag, pol, and env), are removed from the retroviral backbone using genetic engineering techniques known in the art. Accordingly, a minimum retroviral vector comprises from 5′ to 3′: a 5′ long terminal repeat (LTR), a packaging signal, an optional exogenous promoter and/or enhancer, an exogenous gene of interest, and a 3′ LTR. If no exogenous promoter is provided, gene expression is driven by the 5′ LTR, which is a weak promoter and requires the presence of Tat to activate expression. The structural genes can be provided in separate vectors for manufacture of the lentivirus, rendering the produced virions replication-defective. Specifically, with respect to lentivirus, the packaging system may comprise a single packaging vector encoding the Gag, Pol, Rev, and Tat genes, and a third, separate vector encoding the envelope protein Env (usually VSV-G due to its wide infectivity). To improve the safety of the packaging system, the packaging vector can be split, expressing Rev from one vector, Gag and Pol from another vector. Tat can also be eliminated from the packaging system by using a retroviral vector comprising a chimeric 5′ LTR, wherein the U3 region of the 5′ LTR is replaced with a heterologous regulatory element.

The genes can be incorporated into the proviral backbone in several general ways. The most straightforward constructions are ones in which the structural genes of the retrovirus are replaced by a single gene that is transcribed under the control of the viral regulatory sequences within the LTR. Retroviral vectors have also been constructed that can introduce more than one gene into target cells. Usually, in such vectors one gene is under the regulatory control of the viral LTR, while the second gene is expressed either off a spliced message or is under the regulation of its own, internal promoter.

Accordingly, the new gene(s) are flanked by 5′ and 3′ LTRs, which serve to promote transcription and polyadenylation of the virion RNAs, respectively. The term “long terminal repeat” or “LTR” refers to domains of base pairs located at the ends of retroviral DNAs which, in their natural sequence context, are direct repeats and contain U3, R and U5 regions. LTRs generally provide functions fundamental to the expression of retroviral genes (e.g., promotion, initiation and polyadenylation of gene transcripts) and to viral replication. The LTR contains numerous regulatory signals including transcriptional control elements, polyadenylation signals, and sequences needed for replication and integration of the viral genome. The U3 region contains the enhancer and promoter elements. The U5 region is the sequence between the primer binding site and the R region and contains the polyadenylation sequence. The R (repeat) region is flanked by the U3 and U5 regions. In certain embodiments, the R region comprises a trans-activation response (TAR) genetic element, which interacts with the trans-activator (tat) genetic element to enhance viral replication. This element is not required in embodiments wherein the U3 region of the 5′ LTR is replaced by a heterologous promoter.

In certain embodiments, the retroviral vector comprises a modified 5′ LTR and/or 3′ LTR. Modifications of the 3′ LTR are often made to improve the safety of lentiviral or retroviral systems by rendering viruses replication-defective. In specific embodiments, the retroviral vector is a self-inactivating (SIN) vector. As used herein, a SIN retroviral vector refers to a replication-defective retroviral vector in which the 3′ LTR U3 region has been modified (e.g., by deletion or substitution) to prevent viral transcription beyond the first round of viral replication. This is because the 3′ LTR U3 region is used as a template for the 5′ LTR U3 region during viral replication and, thus, the viral transcript cannot be made without the U3 enhancer-promoter. In a further embodiment, the 3′ LTR is modified such that the U5 region is replaced, for example, with an ideal polyadenylation sequence. It should be noted that modifications to the LTRs such as modifications to the 3′ LTR, the 5′ LTR, or both 3′ and 5′ LTRs, are also included in the methods and compositions described herein.

In certain embodiments, the U3 region of the 5′ LTR is replaced with a heterologous promoter to drive transcription of the viral genome during production of viral particles. Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. Typical promoters are able to drive high levels of transcription in a Tat-independent manner. This replacement reduces the possibility of recombination to generate replication-competent virus, because there is no complete U3 sequence in the virus production system.

Adjacent the 5′ LTR are sequences necessary for reverse transcription of the genome (the tRNA primer binding site) and for efficient packaging of viral RNA into particles (the Psi site). As used herein, the term “packaging signal” or “packaging sequence” refers to sequences located within the retroviral genome which are required for encapsidation of retroviral RNA strands during viral particle formation (see e.g., Clever et al., 1995 J. VIROLOGY, 69(4):2101-09). The packaging signal may be a minimal packaging signal (also referred to as the psi [Ψ] sequence) needed for encapsidation of the viral genome.

In certain embodiments, the retroviral vector (e.g., lentiviral vector) further comprises a FLAP. As used herein, the term “FLAP” refers to a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou et al. (2000) CELL, 101:173. During reverse transcription, central initiation of the plus-strand DNA at the cPPT and central termination at the CTS lead to the formation of a three-stranded DNA structure: a central DNA flap. While not wishing to be bound by any theory, the DNA flap may act as a cis-active determinant of lentiviral genome nuclear import and/or may increase the titer of the virus. In particular embodiments, the retroviral vector backbones comprise one or more FLAP elements upstream or downstream of the heterologous genes of interest in the vectors. For example, in particular embodiments, a transfer plasmid includes a FLAP element. In one embodiment, a vector described herein comprises a FLAP element isolated from HIV-1.

In certain embodiments, the retroviral vector (e.g., lentiviral vector) further comprises an export element. In one embodiment, retroviral vectors comprise one or more export elements. The term “export element” refers to a cis-acting post-transcriptional regulatory element, which regulates the transport of an RNA transcript from the nucleus to the cytoplasm of a cell. Examples of RNA export elements include, but are not limited to, the human immunodeficiency virus (HIV) RRE (see e.g., Cullen et al., (1991) J. VIROL. 65: 1053; and Cullen et al., (1991) CELL 58: 423) and the hepatitis B virus post-transcriptional regulatory element (HPRE). Generally, the RNA export element is placed within the 3′ UTR of a gene, and can be inserted as one or multiple copies.

In certain embodiments, the retroviral vector (e.g., lentiviral vector) further comprises a posttranscriptional regulatory element. A variety of posttranscriptional regulatory elements can increase expression of a heterologous nucleic acid, e.g., woodchuck hepatitis virus posttranscriptional regulatory element (WPRE; see Zufferey et al., (1999) J. VIROL., 73:2886); the posttranscriptional regulatory element present in hepatitis B virus (HPRE) (Huang et al., MOL. CELL. BIOL., 5:3864); and the like (Liu et al., (1995), GENES DEV., 9:1766). The posttranscriptional regulatory element is generally positioned at the 3′ end the heterologous nucleic acid sequence. This configuration results in synthesis of an mRNA transcript whose 5′ portion comprises the heterologous nucleic acid coding sequences and whose 3′ portion comprises the posttranscriptional regulatory element sequence. In certain embodiments, vectors described herein lack or do not comprise a posttranscriptional regulatory element such as a WPRE or HPRE, because in some instances these elements increase the risk of cellular transformation and/or do not substantially or significantly increase the amount of mRNA transcript or increase mRNA stability. Therefore, in certain embodiments, vectors described herein lack or do not comprise a WPRE or HPRE as an added safety measure.

Elements directing the efficient termination and polyadenylation of the heterologous nucleic acid transcripts increase heterologous gene expression. Transcription termination signals are generally found downstream of the polyadenylation signal. Accordingly, in certain embodiments, the retroviral vector (e.g., lentiviral vector) further comprises a polyadenylation signal. The term “polyadenylation signal” or “polyadenylation sequence” as used herein denotes a DNA sequence which directs both the termination and polyadenylation of the nascent RNA transcript by RNA polymerase H. Efficient polyadenylation of the recombinant transcript is desirable as transcripts lacking a polyadenylation signal are unstable and are rapidly degraded.

In certain embodiments, a retroviral vector further comprises an insulator element. Insulator elements may contribute to protecting retrovirus-expressed sequences, e.g., therapeutic genes, from integration site effects, which may be mediated by cis-acting elements present in genomic DNA and lead to deregulated expression of transferred sequences (i.e., position effect; see, e.g., Burgess-Beusse et al., (2002) PROC. NATL. ACAD. SCI., USA, 99:16433; and Zhan et al., 2001, HUM. GENET., 109:471). In certain embodiments, the retroviral vector comprises an insulator element in one or both LTRs or elsewhere in the region of the vector that integrates into the cellular genome. Examples of insulators for use in the methods and compositions described herein include, but are not limited to, the chicken β-globin insulator (see Chung et al., (1993). CELL 74:505; Chung et al., (1997) PROC. NATL. ACAD. SCI., USA 94:575; and Bell et al., 1999. CELL 98:387). Examples of insulator elements include, but are not limited to, an insulator from a β-globin locus, such as chicken HS4.

Non-limiting examples of lentiviral vectors include pLVX-EF1alpha-AcGFP1-C1 (Clontech Catalog #631984), pLVX-EF1alpha-IRES-mCherry (Clontech Catalog #631987), pLVX-Puro (Clontech Catalog #632159), pLVX-IRES-Puro (Clontech Catalog #632186), pLenti6/V5-DEST™ (Thermo Fisher), pLenti6.2/V5-DEST™ (Thermo Fisher), pLKO.1 (Plasmid #10878 at Addgene), pLKO.3G (Plasmid #14748 at Addgene), pSico (Plasmid #11578 at Addgene), pLJM1-EGFP (Plasmid #19319 at Addgene), FUGW (Plasmid #14883 at Addgene), pLVTHM (Plasmid #12247 at Addgene), pLVUT-tTR-KRAB (Plasmid #11651 at Addgene), pLL3.7 (Plasmid #11795 at Addgene), pLB (Plasmid #11619 at Addgene), pWPXL (Plasmid #12257 at Addgene), pWPI (Plasmid #12254 at Addgene), EF.CMV.RFP (Plasmid #17619 at Addgene), pLenti CMV Puro DEST (Plasmid #17452 at Addgene), pLenti-puro (Plasmid #39481 at Addgene), pULTRA (Plasmid #24129 at Addgene), pLX301 (Plasmid #25895 at Addgene), pHIV-EGFP (Plasmid #21373 at Addgene), pLV-mCherry (Plasmid #36084 at Addgene), pLionII (Plasmid #1730 at Addgene), pInducer10-mir-RUP-PheS (Plasmid #44011 at Addgene). These vectors can be modified to be suitable for therapeutic use. For example, a selection marker (e.g., puro, EGFP, or mCherry) can be deleted or replaced with a second exogenous gene of interest. Further examples of lentiviral vectors are disclosed in U.S. Pat. Nos. 7,629,153, 7,198,950, 8,329,462, 6,863,884, 6,682,907, 7,745,179, 7,250,299, 5,994,136, 6,287,814, 6,013,516, 6,797,512, 6,544,771, 5,834,256, 6,958,226, 6,207,455, 6,531,123, and 6,352,694, and PCT Publication No. WO2017/091786.

### Adenoviral Vectors

In certain embodiments, the viral vector can be an adenoviral vector. Adenoviruses are medium-sized (90-100 nm), non-enveloped (naked), icosahedral viruses composed of a nucleocapsid and a double-stranded linear DNA genome. The term “adenovirus” refers to any virus in the genus Adenoviridiae including, but not limited to, human, bovine, ovine, equine, canine, porcine, murine, and simian adenovirus subgenera. Typically, an adenoviral vector is generated by introducing one or more mutations (e.g., a deletion, insertion, or substitution) into the adenoviral genome of the adenovirus so as to accommodate the insertion of a non-native nucleic acid sequence, for example, for gene transfer, into the adenovirus.

A human adenovirus can be used as the source of the adenoviral genome for the adenoviral vector. For instance, an adenovirus can be of subgroup A (e.g., serotypes 12, 18, and 31), subgroup B (e.g., serotypes 3, 7, 1 1 , 14, 16, 21 , 34, 35, and 50), subgroup C (e.g., serotypes 1 , 2, 5, and 6), subgroup D (e.g.,, serotypes 8, 9, 10, 13, 15, 17, 19, 20, 22-30, 32, 33, 36-39, and 42-48), subgroup E (e.g., serotype 4), subgroup F (e.g., serotypes 40 and 41 ), an unclassified serogroup (e.g., serotypes 49 and 51), or any other adenoviral serogroup or serotype. Adenoviral serotypes 1 through 51 are available from the American Type Culture Collection (ATCC, Manassas, Virginia). Non-group C adenoviral vectors, methods of producing non-group C adenoviral vectors, and methods of using non- group C adenoviral vectors are disclosed in, for example, U.S. Pat. Nos. 5,801,030, 5,837,511, and 5,849,561, and PCT Publication Nos. WO1997/012986 and WO1998/053087.

Non-human adenovirus (e.g., ape, simian, avian, canine, ovine, or bovine adenoviruses) can be used to generate the adenoviral vector (i.e., as a source of the adenoviral genome for the adenoviral vector). For example, the adenoviral vector can be based on a simian adenovirus, including both new world and old world monkeys (see, e.g., Virus Taxonomy: VHIth Report of the International Committee on Taxonomy of Viruses (2005)). A phylogeny analysis of adenoviruses that infect primates is disclosed in, e.g., Roy et al. (2009) PLOS PATHOG. 5(7):e1000503. A gorilla adenovirus can be used as the source of the adenoviral genome for the adenoviral vector. Gorilla adenoviruses and adenoviral vectors are described in, e.g., PCT Publication Nos.WO2013/052799, WO2013/052811, and WO2013/052832. The adenoviral vector can also comprise a combination of subtypes and thereby be a “chimeric” adenoviral vector.

The adenoviral vector can be replication-competent, conditionally replication-competent, or replication-deficient. A replication-competent adenoviral vector can replicate in typical host cells, i.e., cells typically capable of being infected by an adenovirus. A conditionally-replicating adenoviral vector is an adenoviral vector that has been engineered to replicate under pre-determined conditions. For example, replication-essential gene functions, e.g., gene functions encoded by the adenoviral early regions, can be operably linked to an inducible, repressible, or tissue-specific transcription control sequence, e.g., a promoter. Conditionally-replicating adenoviral vectors are further described in U.S. Pat. No. 5,998,205. A replication-deficient adenoviral vector is an adenoviral vector that requires complementation of one or more gene functions or regions of the adenoviral genome that are required for replication, as a result of, for example, a deficiency in one or more replication-essential gene function or regions, such that the adenoviral vector does not replicate in typical host cells, especially those in a human to be infected by the adenoviral vector.

In some embodiments, the adenoviral vector is replication-deficient, such that the replication- deficient adenoviral vector requires complementation of at least one replication-essential gene function of one or more regions of the adenoviral genome for propagation (e.g., to form adenoviral vector particles). The adenoviral vector can be deficient in one or more replication-essential gene functions of only the early regions (i.e., E1-E4 regions) of the adenoviral genome, only the late regions (i.e., L1-L5 regions) of the adenoviral genome, both the early and late regions of the adenoviral genome, or all adenoviral genes (i.e., a high capacity adenovector (HC-Ad)). See, e.g., Morsy et al. (1998) PROC. NATL. ACAD. SCI. USA 95: 965-976, Chen et al. (1997) PROC. NATL. ACAD. SCI. USA 94: 1645-1650, and Kochanek et al. (1999) HUM. GENE THER. 10(15):2451-9. Examples of replication-deficient adenoviral vectors are disclosed in U.S. Pat. Nos. 5,837,511, 5,851,806, 5,994,106, 6,127,175, 6,482,616, and 7,195,896, and PCT Publication Nos. WO1994/028152, WO1995/002697, WO1995/016772, WO1995/034671, WO1996/022378, WO1997/012986, WO1997/021826, and WO2003/022311.

The replication-deficient adenoviral vector can be produced in complementing cell lines that provide gene functions not present in the replication-deficient adenoviral vector, but required for viral propagation, at appropriate levels in order to generate high titers of viral vector stock. Such complementing cell lines are known and include, but are not limited to, 293 cells (described in, e.g., Graham et al. (1977) J. GEN. VIROL. 36: 59-72), PER.C6 cells (described in, e.g., PCT Publication No. WO1997/000326, and U.S. Patent Nos. 5,994,128 and 6,033,908), and 293-ORF6 cells (described in, e.g., PCT Publication No. WO1995/034671 and Brough et al. (1997) J. VIROL. 71: 9206-9213). Other complementing cell lines to produce the replication-deficient adenoviral vector described herein include complementing cells that have been generated to propagate adenoviral vectors encoding transgenes whose expression inhibits viral growth in host cells (see, e.g., U.S. Patent Publication No. 2008/0233650). Additional complementing cells are described in, for example, U.S. Pat. Nos. 6,677,156 and 6,682,929, and PCT Publication No. WO2003/020879. Formulations for adenoviral vector-containing compositions are further described in, for example, U.S. Pat. Nos. 6,225,289, and 6,514,943, and PCT Publication No. WO2000/034444.

Additional exemplary adenoviral vectors, and/or methods for making or propagating adenoviral vectors are described in U.S. Pat. Nos. 5,559,099, 5,837,511, 5,846,782, 5,851,806, 5,994,106, 5,994,128, 5,965,541, 5,981,225, 6,040,174, 6,020,191, 6,083,716, 6,113,913, 6,303,362, 7,067,310, and 9,073,980.

Commercially available adenoviral vector systems include the ViraPower™ Adenoviral Expression System available from Thermo Fisher Scientific, the AdEasy™ adenoviral vector system available from Agilent Technologies, and the Adeno-X™ Expression System 3 available from Takara Bio USA, Inc.

### Viral Vector Production

Methods for producing viral vectors are known in the art. Typically, a virus of interest is produced in a suitable host cell line using conventional techniques including culturing a transfected or infected host cell under suitable conditions so as to allow the production of infectious viral particles. Nucleic acids encoding viral genes and/or base editor and/or sgRNA can be incorporated into plasmids and introduced into host cells through conventional transfection or transformation techniques. Examples of host cells for production of disclosed viruses include human cell lines, such as HeLa, Hela-S3, HEK293, 911, A549, HER96, or PER-C6 cells. Specific production and purification conditions can vary depending upon the virus and the production system employed.

In certain embodiments, producer cells may be directly administered to a subject, however, in other embodiments, following production, infectious viral particles are recovered from the culture and optionally purified. Typical purification steps may include plaque purification, centrifugation, e.g., cesium chloride gradient centrifugation, clarification, enzymatic treatment, e.g., benzonase or protease treatment, chromatographic steps, e.g., ion exchange chromatography or filtration steps.

### Pharmaceutical Compositions

For therapeutic use, a base editor and sgRNA vector preferably is combined with a pharmaceutically acceptable carrier. The term “pharmaceutically acceptable” as used herein refers to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

The term “pharmaceutically acceptable carrier” as used herein refers to buffers, carriers, and excipients for use in contact with the tissues of human beings and animals without excessive toxicity, irritation, allergic response, or other problem or complication, commensurate with a reasonable benefit/risk ratio. Pharmaceutically acceptable carriers include any of the standard pharmaceutical carriers, such as a phosphate buffered saline solution, water, emulsions (e.g., such as an oil/water or water/oil emulsions), and various types of wetting agents. The compositions also can include stabilizers and preservatives. For examples of carriers, stabilizers and adjuvants, see, e.g., Martin, Remington’s Pharmaceutical Sciences, 15th Ed., Mack Publ. Co., Easton, PA [1975]. Pharmaceutically acceptable carriers include buffers, solvents, dispersion media, coatings, isotonic and absorption delaying agents, and the like, that are compatible with pharmaceutical administration. The use of such media and agents for pharmaceutically active substances is known in the art.

In certain embodiments, a pharmaceutical composition may contain formulation materials for modifying, maintaining or preserving, for example, the pH, osmolarity, viscosity, clarity, color, isotonicity, odor, sterility, stability, rate of dissolution or release, adsorption or penetration of the composition. In such embodiments, formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides; disaccharides; and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants (See Remington’s Pharmaceutical Sciences, 18th ed. (Mack Publishing Company, 1990).

In certain embodiments, a pharmaceutical composition may contain a sustained-or controlled-delivery formulation. Techniques for formulating sustained- or controlled-delivery means, such as liposome carriers, bio-erodible microparticles or porous beads and depot injections, are also known to those skilled in the art. Sustained-release preparations may include, e.g., porous polymeric microparticles or semipermeable polymer matrices in the form of shaped articles, e.g., films, or microcapsules. Sustained release matrices may include polyesters, hydrogels, polylactides, copolymers of L-glutamic acid and gamma ethyl-L-glutamate, poly (2-hydroxyethyl-inethacrylate), ethylene vinyl acetate, or poly-D(-)-3-hydroxybutyric acid. Sustained release compositions may also include liposomes that can be prepared by any of several methods known in the art.

Pharmaceutical compositions containing a base editor and sgRNA expression vector disclosed herein can be presented in a dosage unit form and can be prepared by any suitable method. A pharmaceutical composition should be formulated to be compatible with its intended route of administration. Examples of routes of administration are subretinal or intra vitreal. In certain embodiments, a base editor and/or sgRNA vector is administered by injection. Useful formulations can be prepared by methods known in the pharmaceutical art. For example, see Remington’s Pharmaceutical Sciences, 18th ed. (Mack Publishing Company, 1990). Formulation components suitable for parenteral administration include a sterile diluent such as water for injection, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as EDTA; buffers such as acetates, citrates or phosphates; and agents for the adjustment of tonicity such as sodium chloride or dextrose.

Pharmaceutical formulations can be sterile. Sterilization can be accomplished by any suitable method, e.g., filtration through sterile filtration membranes. Where the composition is lyophilized, filter sterilization can be conducted prior to or following lyophilization and reconstitution.

### Cell Therapy

As an alternative to injection or subretinal injection of vectors or viral particles encoding the based editor and gRNA, cell replacement therapy can be used to prevent, correct or treat IRDs, where the methods of the present disclosure are applied to isolated patient’s cells (ex vivo), which is then followed by the injection of “corrected” cells back into the patient.

In one embodiment, the disclosure provides for introducing one or more vectors encoding base editor and gRNA into a eukaryotic cell. The cell may be a stem cell. Examples of stem cells include pluripotent, multipotent and unipotent stem cells. Examples of pluripotent stem cells include embryonic stem cells, embryonic germ cells, embryonic carcinoma cells and induced pluripotent stem cells (iPSCs). In one aspect, the iPSC is derived from a fibroblast cell.

For the treatment of IRD, the patient’s iPS cells can be isolated and differentiated into retinal pigment epithelium (RPE) cells ex vivo. Patient’s iPS cells or RPE cells characterized by the missense or nonsense mutation in IRD-related gene may be manipulated using methods of the present disclosure in a manner that results in the correction of a mutant allele of the IRD-related gene.

Thus, the present disclosure provides methods for correcting IRD in a subject, wherein the method results in replacement of a mutant allele of the IRD-related gene with the correct allele. The method may comprise administering to the subject a therapeutically effective amount of autologous or allogeneic retinal pigment RPE cells with the corrected allele of the IRD-related gene. Administration of the pharmaceutical preparations comprising RPE cells with the corrected allele of the IRD-related gene may be effective to reduce the severity of symptoms and/or to prevent further deterioration in the subject’s condition. Such administration may be effective to fully restore any vision loss or other symptoms.

“Induced pluripotent stem cells,” commonly abbreviated as iPS cells or iPSCs, refer to a type of pluripotent stem cell artificially prepared from a non-pluripotent cell, typically an adult somatic cell, or terminally differentiated cell, such as a fibroblast, a hematopoietic cell, a myocyte, a neuron, an epidermal cell, or the like, by introducing certain factors, referred to as reprogramming factors.

The present methods may further comprise differentiating the iPS cell to a differentiated cell, for example, an ocular cell.

For example, patient fibroblast cells can be collected from the skin biopsy and transformed into iPS cells. (See, e.g., Luo et al., Generation of induced pluripotent stem cells from skin fibroblasts of a patient with olivopontocerebellar atrophy, Tohoku J. Exp. Med. 2012, 226(2): 151-9). The base editing modification can be done at this stage. The corrected cell clone can be screened and selected by RFLP assay. The corrected cell clone is then differentiated into RPE cells and tested for its RPE-specific markers (e.g., Bestrophin1, RPE65, Cellular Retinaldehyde-binding Protein, and MFRP). Well-differentiated RPE cells can be transplanted autologously back to the donor patient.

In some embodiments, the cell may be autologous or allogeneic to the subject who is administered the cell.

The corrected cells for cell therapy to be administered to a subject (e.g., RPE cells) described in the present disclosure may be formulated with a pharmaceutically acceptable carrier. For example, cells can be administered alone or as a component of a pharmaceutical formulation. The cells (e.g., RPE cells) can be administered in combination with one or more pharmaceutically acceptable sterile isotonic aqueous or nonaqueous solutions (e.g., balanced salt solution (BSS)), dispersions, suspensions or emulsions, or sterile powders which may be reconstituted into sterile injectable solutions or dispersions just prior to use, which may contain antioxidants, buffers, bacteriostats, solutes or suspending or thickening agents.

The present system may be delivered into the retina of a subject. The present system may be administered through injections, such as subretinal or intravitreal injections.

The corrected cells (e.g., RPE cells) may be delivered in a pharmaceutically acceptable ophthalmic formulation by intraocular injection. Concentrations for injections may be at any amount that is effective and nontoxic. The pharmaceutical preparations of the cells of the present disclosure for treatment of a patient may be formulated at doses of at least about 10.sup.4 cells/mL. The cell preparations for treatment of a patient can be formulated at doses of at least or about 10³, 10⁴, 10⁵, 10⁶, 10⁷, 10⁸, 10⁹, or 10¹⁰ cells/mL.

Subjects, which may be treated according to the present disclosure, include all animals which may benefit from the present invention. Such subjects include mammals, preferably humans (infants, children, adolescents and/or adults), but can also be an animal such as dogs and cats, farm animals such as cows, pigs, sheep, horses, goats and the like, and laboratory animals (e.g., rats, mice, guinea pigs, and the like).

### Therapeutic Uses

The compositions and methods disclosed herein can be used to treat any inherited retinal disease (IRD) to permanently rescue the function of a key vision related protein disabled by mutations, or to correct dominant or recessive alleles for which gene augmentation may not be effective. The IRD can include chorioretinal atrophy or degeneration, cone or cone-rod dystrophy, congenital stationary night blindness, Leber congenital amaurosis, macular degeneration, ocular-retinal developmental disease, optic atrophy, retinitis pigmentosa, syndromic/systemic diseases with retinopathy, sorsby macular dystrophy, age-related macular degeneration, doyne honeycomb macular disease, juvenile macular degeneration, Stargardt disease, or retinitis pigmentosis.

In some embodiments, the methods described herein can be used for arresting progression of or ameliorating vision loss associated with retinitis pigmentosa (RP) associated with a missense or nonsense mutation in the subject.

Vision loss may include decrease in peripheral vision, central (reading) vision, night vision, day vision, loss of color perception, loss of contrast sensitivity, or reduction in visual acuity. The methods of the present disclosure can also be used to prevent, or arrest photoreceptor function loss, or increase photoreceptor function in the subject.

RP is diagnosed in part, through an examination of the retina. The eye exam usually reveals abnormal, dark pigment deposits that streak the retina. Additional tests for diagnosing RP include electroretinogram (ERG) and visual field testing.

Methods for measuring or assessing visual function, retinal function (such as responsiveness to light stimulation), or retinal structure in a subject are well known to one of skill in the art. See, e.g., Kanski’s Clinical Ophthalmology: A Systematic Approach, Edition 8, Elsevier Health Sciences, 2015. Methods for measuring or assessing retinal response to light include may include detecting an electrical response of the retina to a light stimulus. This response can be detected by measuring an electroretinogram (ERG; for example, full-field ERG, multifocal ERG, or ERG photostress test), visual evoked potential, or optokinetic nystagmus (see, e.g., Wester et al., Invest. Ophthalmol. Vis. Sci. 48:4542-4548, 2007). Furthermore, retinal response to light may be measured by directly detecting retinal response (for example by use of a microelectrode at the retinal surface). ERG has been extensively described by Vincent et al. Retina, 2013; 33(1):5-12. Thus, methods of the present disclosure can be used to improve visual function, retinal function (such as responsiveness to light stimulation), retinal structure, or any other clinical symptoms or phenotypic changes associated with ocular diseases in subjects afflicted with ocular disease.

In one embodiment, the methods described herein can be used to prevent the development and progression of an IRD. For example, a patient may be a carrier of an IRD related mutation, but the phenotypic expression of a disease has not been yet manifested, although the genomic defect has been identified by screening. The methods described herein may be applied to such patient to prevent the onset of disease.

The methods described herein can be used to prevent, correct, or treat any IRDs that arise from mutated gene. Thus, all the methods described herein can be used to prevent, correct, or treat an IRD that arise due to the presence of autosomal dominant mutations and autosomal recessive mutations and, hence, treat an autosomal dominant IRD or autosomal recessive IRD.

Examples of autosomal dominant and autosomal recessive IRD-related diseases are disclosed below. In all cases where accession numbers are used, the accession numbers refer to one embodiment of the gene which may be used with the methods of the present disclosure. In one embodiment, the accession numbers are NCBI (National Center for Biotechnology Information) reference sequence (RefSeq) numbers.

For example, the autosomal dominant IRD-related gene in retinitis pigmentosa may include, but are not limited to, ARL3(NC_000010.11 (102673727 ... 102714433, complement)), BEST1 (e.g., NG_009033.1), CA4 (NG_012050.1), CRX (NG_008605.1), FSCN2 (NG_015964.1), GUCA1B (NG_016216.1), HK1 (NG_012077.1), IMPDH1 (NG_009194.1), KLHL7 (NG_016983.1), NR2E3 (NG_009113.2), NRL (NG_011697.1), PRPF3 (NG_008245.1), PRPF4 (NG_034225.1), PRPF6 (NG_029719.1), PRPF8 (NG_009118.1), PRPF31 (NG_009759.1), PRPH2 (NG_009176.1), RDH12 (NG_008321.1), RHO (NG_009115.1), ROM1 (NG_009845.1), RP1 (NG_009840.1), RP9 (NG_012968.1), RPE65 (NG_008472.1), SEMA4A (NG_027683.1), SNRNP200 (NG_016973.1), SPP2 (NG_008668.1), and TOPORS (NG_017050.1). Genes and mutations causing autosomal dominant retinitis pigmentosa are discussed in detail by Daiger et al. (Cold Spring Harb Perspect Med. 2014 Oct. 10; 5(10)).

Another type of the autosomal dominant IRD-related gene is autosomal dominant chorioretinal atrophy or degeneration-related gene, which may include: PRDM13 (NC_000006.12 (99606774 ... 99615578)), RGR (NG_009106.1), and TEAD1 (NG_021302.1).

Another example of the autosomal dominant IRD-related gene is autosomal dominant cone or cone-rod dystrophy-related gene, which can include: AIPL1 (NG_008474.1), CRX (NG_008605.1), GUCA1A (NG_009938.1), GUCY2D (NG_009092.1), PITPNM3 (NG_016020.1), PROM1 (NG_011696.1), PRPH2 (NG_009176.1), RIMS1 (NG_016209.1), SEMA4A (NG_027683.1), and UNC119 (NG_012302.1).

In one embodiment, the autosomal dominant IRD-related gene is autosomal dominant congenital stationary night blindness-related gene, including: GNAT1 (NG_009831.1), PDE6B (NG_009839.1), and RHO (NG_009115.1).

Another type of the autosomal dominant IRD-related gene is autosomal dominant Leber congenital amaurosis-related gene, which may include: CRX(NG_008605.1), (NG_009194.1), and OTX2(NG_008204.1).

Another example of the autosomal dominant IRD-related gene is autosomal dominant macular degeneration-related gene, which can include: BEST1(NG_009033.1), C1QTNF5 (NG_012235.1), CTNNA1 (NC_000005.10 (138753396 ... 138935034)), EFEMP1 (NG_009098.1), ELOVL4 (NG_009108.1), FSCN2 (NG_015964.1), GUCA1B (NG_016216.1), HMCN1 (NG_011841.1), IMPG1 (NG_041812.1), OTX2 (NG_008204.1), PRDM13 (NC_000006.12 (99606774 ... 99615578)), PROM1 (NG_011696.1), PRPH2 (NG_009176.1), RP1L1 (NG_028035.1), and TIMP3(NG_009117.1).

In one embodiment, the autosomal dominant IRD-related gene is autosomal dominant ocular retinal developmental disease-related gene such as VCAN(NG_012682.1).

In another embodiment, the autosomal dominant IRD-related gene is autosomal dominant optic atrophy-related gene, including: MFN2 (NG_007945.1), NR2F1 (NG_034119.1), and OPA1 (NG_011605.1).

In one embodiment, the autosomal dominant IRD-related gene is autosomal dominant syndromic/systemic disease with retinopathy-related gene, including: ABCC6 (NG_007558.2), ATXN7 (NG_008227.1), COL11A1 (NG_008033.1), COL2A1 (NG_008072.1), JAG1 (NG_007496.1), KCNJ13 (NG_016742.1), KIF11 (NG_032580.1), MFN2 (NG_007945.1), OPA3 (NG_013332.1), PAX2 (NG_008680.2), TREX1 (NG_009820.1), and VCAN (NG_012682.1).

Another example of the autosomal dominant IRD-related gene is autosomal dominant retinopathy-related gene, including: BEST1 (NG_009033.1), CAPN5 (NG_033002.1), CRB1 (NG_008483.2), FZD4 (NG_011752.1), ITM2B (NG_013069.1), LRP5 (NG_015835.1), MAPKAPK3 (NC_000003.12(50611862 ... 50649297)), MIR204 (NR 029621.1), OPN1SW (NG_009094.1), RB1 (NG_009009.1), TSPAN12 (NG_023203.1), and ZNF408 (NC_000011.10 (46700767 ... 46705916).

One type of the autosomal recessive IRD-related gene is congenital stationary night-related gene, including: CABP4(NG_021211.1), GNAT1(NG_009831.1), GNB3 (NG_009100.1), GPR179(NG_032655.2), GRK1(NC_000013.11(113667279 ... 113671659)), GR M6(NG_008105.1), LRIT3(NG_033249.1), RDH5(NG_008606.1), SAG(NG_009116.1), SLC24 Al(NG_031968.2), and TRPM1(NG_016453.2).

Another type of the autosomal recessive IRD-related gene is bardet-biedl syndrome-related gene, including: ADIPOR1 (NC_000001.1 (202940825 ... 202958572, complement)), ARL6 (NG_008119.2), BBIP1 (NG_041778.1), BBS1 (NG_009093.1), BBS2 (NG_009312.1), BBS4 (NG_009416.2), BBS5 (NG_011567.1), BBS7 (NG_009111.1), BBS9 (NG_009306.1), BBS10 (NG_016357.1), BBS12 (NG_021203.1), C8orf37 (NG_032804.1), CEP290 (NG_008417.1), IFT172 (NG_034068.1), IFT27 (NG_034205.1), INPP5E (NG_016126.1), KCNJ13 (NG_016742.1), LZTFL1 (NG_033917.1), MKKS (NG_009109.1), MKS1 (NG_013032.1), NPHP1 (NG_008287.1), SDCCAG8 (NG_027811.1), TRIM32 (NG_011619.1), and TTC8 (NG_008126.1).

One example of the autosomal recessive IRD-related gene is cone or cone-rod dystrophy-related gene, including, but not limited to, ABCA4(NG_009073.1), ADAMS (NG_016335.1), ATF6 (NG_029773.1), C21orf2 (NG_032952.1), C8orf37 (NG_032804.1), CACNA2D4 (NG_012663.1), CDHR1 (NG_028034.1), CERKL (NG_021178.1), CNGA3 (NG_009097.1), CNGB3 (NG_016980.1), CNNM4 (NG_016608.1), GNAT2 (NG_009099.1), KCNV2 (NG_012181.1), PDE6C (NG_016752.1), PDE6H (NG_016859.1), POC1B (NG_041783.1), RAB28 (NG_033891.1), RAX2 (NG_011565.1), RDH5 (NG_008606.1), RPGRIP1 (NG_008933.1), and TTLL5(NG_016974.1).

Another example of the autosomal recessive IRD-related gene is deafness (alone or syndromic)-related gene including: CDH23(NG_008835.1), CIB2(NG_033006.1), DFNB31 (NG_016700.1), MYO7A (NG_009086.1), PCDH15 (NG_009191.2), PDZD7 (NG_028030.1), and USH1C(NG_011883.1).

In one embodiment, the autosomal recessive IRD-related gene is Leber congenital amaurosis-related gene, including: AIPL1(NG_008474.1), CABP4(NG_021211.1), CEP290 (NG_008417.1), CLUAP1 (NC_000016.10(3500945 ... 3539048)), CRB1 (NG_008483.2), CRX (NG_008605.1), DTHD1 (NG_032962.1), GDF6 (NG_008981.1), GUCY2D (NG_009092.1), IFT140 (NG_032783.1), IQCB1 (NG_015887.1), KCNJ13 (NG_016742.1), LCAS (NG_016011.1), LRAT (NG_009110.1), NMNAT1 (NG_032954.1), PRPH2 (NG_009176.1), RD3 (NG_013042.1), RDH12 (NG_008321.1), RPE65 (NG_008472.1), RPGRIP1 (NG_008933.1), SPATA7 (NG_021183.1), and TULP1 (NG_009077.1).

In another embodiment, the autosomal recessive IRD-related gene is optic atrophy-related gene, including: RTN4IP1(NC_000006.12 (106571028 ... 106630500, complement)), SLC25A46 (NC_000005.10 (110738136 ... 110765161)), and TMEM126A(NG_017157.1).

One example of the autosomal recessive IRD-related gene is retinitis pigmentosa-related gene, including: ABCA4 (NG_009073.1), AGBLS (NC_000002.12 (27051423 ... 27070622)), ARL6 (NG_008119.2), ARL2BP (NG_033905.1), BBS1 (NG_009093.1), BBS2 (NG_009312.1), BEST1 (NG_009033.1), C2orf71 (NG_021427.1), C8orf37 (NG_032804.1), CERKL (NG_021178.1), CLRN1 (NG_009168.1), CNGA1 (NG_009193.1), CNGB1 (NG_016351.1), CRB1 (NG_008483.2), CYP4V2 (NG_007965.1), DHDDS (NG_029786.1), DHX38 (NG_034207.1), EMC1 (NG_032948.1), EYS (NG_023443.2), FAM161A (NG_028125.1), GPR125 (NC_000004.12 (22387374 ... 22516058, complement)), HGSNAT(NG_009552.1), IDH3B (NG_012149.1), IFT140 (NG_032783.1), IFT172 (NG_034068.1), IMPG2 (NG_028284.1), KIAA1549 (NG_032965.1), KIZ (NG_033122.1), LRAT (NG_009110.1), MAK (NG_030040.1), MERTK (NG_011607.1), MVK (NG_007702.1), NEK2 (NG_029112.1), NEUROD1 (NG_011820.1), NR2E3 (NG_009113.2), NRL (NG_011697.1), PDE6A (NG_009102.1), PDE6B (NG_009839.1), PDE6G (NG_009834.1), POMGNT1 (NG_009205.2), PRCD (NG_016702.1), PROM1 (NG_011696.1), RBP3(NG_029718.1), RGR(NG_009106.1), RHO(NG_009115.1), RLBP1(NG_008116.1), RP1(NG_009840.1), RP1L1(NG 028035.1), RPE65(NG_008472.1), SAG(NG_009116.1), SLC7A14(NG_034121.1), SPATA7(NG_021183.1), TTC8(NG_008126.1), TULP1(NG_009077 0.1), USH2A(NG_009497.1), ZNF408(NC_000011.10 (46700767 ... 46705916)), and ZNF513 (NG_028219.1).

Another example of the autosomal recessive IRD-related gene is syndromic/systemic disease with retinopathy-related gene, including: ABCC6(NG_007558.2), ABHD12 (NG_028119.1), ACBDS (NG_032960.2), ADAMTS18(NG_031879.1), ADIPOR1 (NC_000001.11(202940825 ... 202958572, complement)), AHI1(NG_008643.1), ALMS1 (NG_011690.1), CC2D2A(NG_013035.1), CEP164(NG_033032.1), CEP290 (NG_008417.1), CLN3(NG_008654.2), COL9A1(NG_011654.1), CSPP1(NG_034100.1), ELOVL4(NG_009108.1), EXOSC2 (NC_000009.12 (130693760 ... 130704894)), FLVCR1(NG_028131.1), FLVCR1 (NG_028131.1), GNPTG(NG_016985.1), HARS(NG_032158.1), HGSNAT(NG_009552.1), H MX1(NG_013062.2), IFT140(NG_032783.1), INPP5E(NG_016126.1), INVS(NG_008316.1), IQ CB1(NG_015887.1), LAMA1(NG_034251.1), LRP5(NG_015835.1), MKS1(NG_013032.1), M TTP(NG_011469.1), NPHP1(NG_008287.1), NPHP3(NG_008130.1), NPHP4(NG_011724.2), 0 PA3(NG_013332.1), PANK2(NG_008131.3), PCYT1A(NG_042817.1), PEX1(NG_008341.1), PEX2(NG_008371.1), PEX7(NG_008462.1), PHYH(NG_012862.1), PLK4(NG_041821.1), PNP LA6(NG_013374.1), POC1B(NG_041783.1), PRPS1(NG_008407.1), RDH11(NG_042282.1), RPGRIP1L(NG_008991.2), SDCCAG8(NG_027811.1), SLC25A46(NC_000005.10(110738136 ... 110765161)), TMEM237(NG_032049.1), TRNT1(NG_041800.1), TTPA(NG_016123.1), TUB(NG_029912.1), TUBGCP4(NG_042168.1), TUBGCP6(NG_032160.1), WDPCP(NG_028144.1), WDR19(NG_031813.1), WFS1(NG_011700.1), and ZNF423(NG_032972.2).

One type of the autosomal recessive IRD-related gene is usher syndrome-related gene, including: ABHD12(NG_028119.1), CDH23(NG_008835.1), CEP250 (NC_000020.11 (35455139 ... 35517531)), CIB2(NG_033006.1), CLRN1(NG_009168.1), DFNB31(NG _016700.1), GPR98(NG_007083.1), HARS(NG_032158.1), MYO7A(NG_009086.1), PCDH15(NG_00919 1.2), USH1C(NG_011883.1), USH1G(NG_007882.1), and USH2A(NG_009497.1).

Another type of the autosomal recessive IRD-related gene is retinopathy-related gene, including: BEST1(NG_009033.1), C12orf65(NG_027517.1), CDH3(NG_009096.1), CNGA3NG_009097.1), CNGB3(NG_016980.1), CNNM4(NG_016608.1), CYP4V2(NG_00796 5.1), LRP5(NG_015835.1), MFRP(NG_012235.1), MVK(NG_007702.1), NBAS (NG_032964.1), NR2E3 (NG_009113.2), OAT(NG_008861.1), PLA2G5(NG_032045.1), PROM1(NG_011696.1), RBP4(NG_009104.1), RGS9(NG_013021.1), RGS9BP (NG_016751.1), and RLBP1 (NG_008116.1).

Yet another type of the autosomal recessive IRD-related gene is macular degeneration-related gene, including: ABCA4(NG_009073.1), CFH(NG_007259.1), DRAM2 (NC_000001.11 (111117332 ... 111140216, complement)), IMPG1(NG_041812.1), and MFSD8(NG_008657.1).

In addition to being used for the prevention, correctness, or treatment of autosomal dominant and recessive IRDs, the methods describe herein can be used to prevent, correct, or treat any X-linked IRDs. Thus, all the methods described here as applicable to autosomal dominant and recessive IRDs and autosomal dominant and recessive genes or fragments can be adopted for use in the treatment of X-linked diseases.

Furthermore, the methods described herein can be used to prevent, correct, or treat IRDs that arise due to the presence of X-linked mutation. Examples of such IRDs include: X-linked cone or cone-rod dystrophy, X-linked congenital stationary night blindness, X-linked macular degeneration, X-linked retinitis pigmentosa, X-linked syndromic/systemic diseases with retinopathy, X-linked optic atrophy, and X-linked retinopathies. According to the methods described here, X-linked IRD-related gene is corrected and can in part or fully restore the function of a wild-type gene.

One example of the X-linked IRD-related gene is cone or cone-rod dystrophy-related gene, including: CACNA1F(NG_009095.2) and RPGR(NG_009553.1).

Another example of the X-linked IRD-related gene is congenital stationary night blindness-related gene, including: CACNA1F(NG_009095.2) and NYX(NG_009112.1).

In one embodiment, the X-linked IRD-related gene is macular degeneration-related gene, such as RPGR(NG_009553.1).

In another embodiment, the X-linked IRD-related gene is optic atrophy-related gene, such as TIMM8A(NG_011734.1).

One type of the X-linked IRD-related gene is retinitis pigmentosa-related gene, including: OFD1 (NG_008872.1), RP2 (NG_009107.1), and RPGR (NG_009553.1).

Another type of the X-linked IRD-related gene is syndromic/systemic disease with retinopathy-related gene, including: OFD1(NG_008872.1) and TIMM8A(NG_011734.1).

Yet another example of the X-linked disease-related gene is retinopathy-related gene, including, CACNA1F (NG_009095.2), CHM (NG_009874.2), DMD (NG_012232.1), NDP (NG_009832.1), OPN1LW (NG_009105.2), OPN1MW(NG_011606.1), PGK1(NG_008862.1), and RS1(NG_008659.3).

### Base Editing Efficiency

Some aspects of the disclosure are based on the recognition that any of the base editors and gRNA provided herein are capable of modifying a specific nucleotide base without generating a significant proportion of indels. An “indel”, as used herein, refers to the insertion or deletion of a nucleotide base within a nucleic acid. Such insertions or deletions can lead to frame shift mutations within a coding region of a gene. In some embodiments, it is desirable to generate base editors that efficiently modify (e.g., mutate or deaminate) a specific nucleotide within a nucleic acid, without generating a large number of insertions or deletions (i.e., indels) in the nucleic acid. In certain embodiments, any of the base editors provided herein are capable of generating a greater proportion of intended modifications (e.g., point mutations or deaminations) versus indels. In some embodiments, the base editors provided herein are capable of generating a ratio of intended point mutations to indels that is greater than 1:1.

In some embodiments, the base editors and gRNA provided herein are capable of generating a ratio of intended point mutations to indels that is at least 1.5: 1, at least 2: 1, at least 2.5: 1, at least 3: 1, at least 3.5: 1, at least 4: 1, at least 4.5: 1, at least 5: 1, at least 5.5: 1, at least 6: 1, at least 6.5: 1, at least 7: 1, at least 7.5: 1, at least 8: 1, at least 10: 1, at least 12: 1, at least 15: 1, at least 20: 1, at least 25: 1, at least 30: 1, at least 40: 1, at least 50: 1, at least 100: 1, at least 200: 1, at least 300: 1, at least 400: 1, at least 500: 1, at least 600: 1, at least 700: 1, at least 800: 1, at least 900: 1, or at least 1000: 1, or more. The number of intended mutations and indels may be determined using any suitable method, for example the methods used in the below Examples. In some embodiments, to calculate indel frequencies, sequencing reads are scanned for exact matches to two bp sequences that flank both sides of a window in which indels might occur. If no exact matches are located, the read is excluded from analysis. If the length of this indel window exactly matches the reference sequence the read is classified as not containing an indel. If the indel window is two or more bases longer or shorter than the reference sequence, then the sequencing read is classified as an insertion or deletion, respectively.

In some embodiments, the base editors and gRNA provided herein are capable of limiting formation of indels in a region of a nucleic acid. In some embodiments, the region is at a nucleotide targeted by a base editor and gRNA or a region within 2, 3, 4, 5, 6, 7, 8, 9, or 10 nucleotides of a nucleotide targeted by a base editor and gRNA. In some embodiments, any of the base editors provided herein are capable of limiting the formation of indels at a region of a nucleic acid to less than 1%, less than 1.5%, less than 2%, less than 2.5%, less than 3%, less than 3.5%, less than 4%, less than 4.5%, less than 5%, less than 6%, less than 7%, less than 8%, less than 9%, less than 10%, less than 12%, less than 15%, or less than 20%. The number of indels formed at a nucleic acid region may depend on the amount of time a nucleic acid (e.g., a nucleic acid within the genome of a cell) is exposed to a base editor. In some embodiments, a number or proportion of indels is determined after at least 1 hour, at least 2 hours, at least 6 hours, at least 12 hours, at least 24 hours, at least 36 hours, at least 48 hours, at least 3 days, at least 4 days, at least 5 days, at least 7 days, at least 10 days, or at least 14 days of exposing a nucleic acid (e.g., a nucleic acid within the genome of a cell) to a base editor.

In some embodiment, the base editor and gRNA can be selected by measuring the base editing efficiency of candidate base editors and gRNA in vitro using a cell or population of cells having the point mutation associated with the IRD related gene. The cells can be autologous cells from a subject being treated or allogeneic cells that have been genetically modified to integrate IRD related gene.

In some embodiments, mouse embryonic fibroblasts, such as NIH3T3 cells can be transducted with a vector comprising the IRD related gene with a nonsense mutation and optionally a reporter molecule. The genetically modified NIH3T3 cells can then be transfected with selected vectors encoding a selected base editor and sgRNA and the expression of the corrected gene can be measured by, for example, Western blot analysis to determine rescue of gene expression of the IRD-related gene. The correction rate and base editing efficiency of the selected base editor and gRNA can then be determined using sequencing analysis.

By way of example, NIH3T3-RPE65 (rd12) stable cell lines were generated by transduction of NIH3T3 cells with retrovirus obtained from Phoenix-Eco cells transfected with pMXs-RPE65(rd12)-IRES-GFP. To make RPE65 expression vectors, rd12 mouse Rpe65 cDNA sequence, flanked by EcoRI and NotI, were cloned into the multiple cloning site of pMXs-IRES-GFP. The downstream sequence of the internal ribosomal entry site (IRES) and enhanced green fluorescence protein (EGFP) allows co-expression of RPE65 and EGFP, thereby enabling cell sorting by flow cytometry. The NIH3T3-RPE65 (rd12) cells were seeded on a 24-well plate and transfected with an ABE-expression plasmid and sgRNA-expression plasmid using Lipofectamine. The cell were harvested and RPE65 expression was subsequently determined by Western blot analysis. Deep sequencing analysis was used to quantify the correction rate and base editing efficiency.

It will be appreciated that the cells used for in vitro selection or screening of base editors and gRNA need not be limited to mouse embryonic fibroblasts and that other cells, such as fibroblasts obtained from the subject or induced pluripotent cells, can used to select and screen base editors and gRNA having the desired correction rate and base editing efficiency. For example, fibroblasts from a subject to be treated can be isolated and optionally and transformed into iPS cells. The isolated fibroblast and/or iPS can transfected with the selected base editor and gRNA to determine correction rate and base efficiency.

In some embodiments, the determined correction rate and base editing efficiency of the selected base editor and gRNA can be compared to a control correction rate to select base editors and gRNA for use in treating a subject.

In some embodiments, the selected base editors and gRNA identified using in vitro cell assays described herein can increase the expression of a visual cycle protein, such as RPE65, and at amount effective to enhance vision and/or restore normal vision. In certain embodiments of any of the foregoing methods, the selected base editors and gRNA can increase the expression of a visual cycle protein (e.g., RPE65) associated with a nonsense or missense mutation of an IRD (e.g., LCA). For example, in certain embodiments, a cell contains about 5%, about 10%, about 20%, about 30%, about 40%, about 50%, about 60%, about 70%, about 80%, or about 90% of the gene product relative to a cell without the missense or nonsense mutation. In certain embodiments, the cell contains from about 5% to about 80%, about 5% to about 60%, about 5% to about 40%, about 5% to about 20%, about 5% to about 10%, about 10% to about 80%, about 10% to about 60%, about 10% to about 40%, about 10% to about 20%, about 20% to about 80%, about 20% to about 60%, about 20% to about 40%, about 40% to about 80%, about 40% to about 60%, or about 60% to about 80% of the gene product relative to a cell without the missense or nonsense mutation. In certain embodiments, there is no detectable gene product in the cell. Gene product amount or expression may be measured by any method known in the art, for example, Western blot or ELISA.

In certain embodiments, wherein the gene is a IRD related gene (e.g., RPE65 gene) with a nonsense mutation (e.g., C130T) that encodes visual cycle protein, the base editors described herein can be selected to increases the visual cycle protein (e.g., RPE65) expression in a cell by at least about 4%, about 5%, about 6%, about 7 %, about 8%, about 9%, about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 250%, about 300%, about 350%, about 400%, about 450%, about 500%, about 600%, about 700%, about 800%, about 900%, or about 1000% relative to a cell, tissue, or subject without the nonsense mutation.

In certain embodiments, the base editors and gRNA can be selected that increase visual cycle protein (e.g., RPE65) expression in a cell from about 20% to about 200%, about 20% to about 180%, about 20% to about 160%, about 20% to about 140%, about 20% to about 120%, about 20% to about 100%, about 20% to about 80%, about 20% to about 60%, about 20% to about 40%, about 40% to about 200%, about 40% to about 180%, about 40% to about 160%, about 40% to about 140%, about 40% to about 120%, about 40% to about 100%, about 40% to about 80%, about 40% to about 60%, about 60% to about 200%, about 60% to about 180%, about 60% to about 160%, about 60% to about 140%, about 60% to about 120%, about 60% to about 100%, about 60% to about 80%, about 80% to about 200%, about 80% to about 180%, about 80% to about 160%, about 80% to about 140%, about 80% to about 120%, about 80% to about 100%, about 100% to about 200%, about 100% to about 180%, about 100% to about 160%, about 100% to about 140%, about 100% to about 120%, about 120% to about 200%, about 120% to about 180%, about 120% to about 160%, about 120% to about 140%, about 140% to about 200%, about 140% to about 180%, about 140% to about 160%, about 160% to about 200%, about 160% to about 180%, or about 180% to about 200% relative to a cell, tissue, or subject with the RPE65 mutation.

In other embodiments, the selected base editors and gRNA identified using in vitro cell assays described herein can increase the expression of a visual cycle protein, such as RPE65, at an amount effective to enhance vision and/or restore normal vision. In certain embodiments of any of the foregoing methods, the selected base editors and gRNA can increase the expression of a visual cycle protein (e.g., RPE65) associated with a nonsense or missense mutation of an IRD (e.g., LCA) in the retina or retinal pigment epithelium of the subect. For example, in certain embodiments, a retina cell or retinal pigment epithelium cell of the subject expresses about 5%, about 10%, about 20%, about 30%, about 40%, about 50%, about 60%, about 70%, about 80%, or about 90% of the gene product relative to a cell without the missense or nonsense mutation. In certain embodiments, the retina cell or retinal pigment epithelium cell expresses from about 5% to about 80%, about 5% to about 60%, about 5% to about 40%, about 5% to about 20%, about 5% to about 10%, about 10% to about 80%, about 10% to about 60%, about 10% to about 40%, about 10% to about 20%, about 20% to about 80%, about 20% to about 60%, about 20% to about 40%, about 40% to about 80%, about 40% to about 60%, or about 60% to about 80% of the gene product relative to a cell without the missense or nonsense mutation. In certain embodiments, there is no detectable gene product in the retina cell or retinal pigment epithelium cell. Gene product amount or expression may be measured by any method known in the art, for example, Western blot or ELISA.

In certain embodiments, where the gene is a RPE65 gene with a nonsense mutation (e.g., C130T), the selected base editors and gRNA described herein can increase RPE65 expression in a retina cell or retinal pigment epithelium cell by at least about 4%, about 5%, about 6%, about 7 %, about 8%, about 9%, about 10%, about 20%, about 30%, about 40%, about 50%, about 60%, about 70%, about 80%, about 90%, about 100%, about 110%, about 120%, about 130%, about 140%, about 150%, about 160%, about 170%, about 180%, about 190%, about 200%, about 250%, about 300%, about 350%, about 400%, about 450%, about 500%, about 600%, about 700%, about 800%, about 900%, or about 1000% relative to a retina cell or retinal pigment epithelium cell without the nonsense mutation.

In certain embodiments, the method increases RPE65 expression in a retina cell or retinal pigment epithelium cell by from about 20% to about 200%, about 20% to about 180%, about 20% to about 160%, about 20% to about 140%, about 20% to about 120%, about 20% to about 100%, about 20% to about 80%, about 20% to about 60%, about 20% to about 40%, about 40% to about 200%, about 40% to about 180%, about 40% to about 160%, about 40% to about 140%, about 40% to about 120%, about 40% to about 100%, about 40% to about 80%, about 40% to about 60%, about 60% to about 200%, about 60% to about 180%, about 60% to about 160%, about 60% to about 140%, about 60% to about 120%, about 60% to about 100%, about 60% to about 80%, about 80% to about 200%, about 80% to about 180%, about 80% to about 160%, about 80% to about 140%, about 80% to about 120%, about 80% to about 100%, about 100% to about 200%, about 100% to about 180%, about 100% to about 160%, about 100% to about 140%, about 100% to about 120%, about 120% to about 200%, about 120% to about 180%, about 120% to about 160%, about 120% to about 140%, about 140% to about 200%, about 140% to about 180%, about 140% to about 160%, about 160% to about 200%, about 160% to about 180%, or about 180% to about 200% relative to a retina cell or retinal pigment epithelium cell with the RPE65 mutation.

Further, the example below shows the feasibility and efficacy of base-editing as a treatment approach for a wide range of inherited retinal diseases (IRDs) caused by different mutations, rather than a therapy for this single mutation. Previous studies have demonstrated therapeutic base-editing in the mouse liver and muscle, but this disclosure represents the first application of a base editor approach in the eye with significant rescue of visual function.

The significance lies in the fact that in vivo base editing in the eye showed a remarkable rescue of visual function and correction of the pathogenic mutation with minimal off-target effects. Such a level of vision restoration has not been achieved by any other pharmacological or genome-editing approach. Given that gene transfer via subretinal injection is already performed in the clinical setting, personalized gene therapy based on base editor delivery can be a new treatment paradigm for a wide range of inherited retinal diseases. This also provides a potential framework for optimizing base editing gene therapy for any possible mutation by screening for an effective base editor and gRNA using an in vitro cell line with the same genetic background and translating it into a therapeutic viral delivery platform. As such, base editing outcomes may be tailored to the unique needs of a patient. Such editing strategies may first be optimized if necessary using fibroblast cells isolated from the patient in question, as we performed for our mouse model.

It will be appreciated that the methods and compositions described herein can be used alone or in combination with other therapeutic agents and/or modalities. The term administered “in combination,” as used herein, is understood to mean that two (or more) different treatments are delivered to the subject during the course of the subject’s affliction with the disorder, such that the effects of the treatments on the patient overlap at a point in time. In certain embodiments, the delivery of one treatment is still occurring when the delivery of the second begins, so that there is overlap in terms of administration. This is sometimes referred to herein as “simultaneous” or “concurrent delivery.” In other embodiments, the delivery of one treatment ends before the delivery of the other treatment begins. In certain embodiments of either case, the treatment is more effective because of combined administration. For example, the second treatment is more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In certain embodiments, delivery is such that the reduction in a symptom, or other parameter related to the disorder is greater than what would be observed with one treatment delivered in the absence of the other. The effect of the two treatments can be partially additive, wholly additive, or greater than additive. The delivery can be such that an effect of the first treatment delivered is still detectable when the second is delivered.

The function and advantage of these and other embodiments of the present invention will be more fully understood from the Examples below. The following Examples are intended to illustrate the benefits of the present invention and to describe particular embodiments, but are not intended to exemplify the full scope of the invention. Accordingly, it will be understood that the Examples are not meant to limit the scope of the invention.

### Example 1

In this Example we show that base editors can be used to target mutations associated with inherited retinal diseases (IRDs) and restore visual function in subjects with IRDs.

### Methods

### Mice

The pigmented rd12 mice and C57BL/6J mice were purchased from The Jackson Laboratory (Jackson Laboratory; 005379 and 000664, respectively). All mice were housed in the vivarium at the University of California, Irvine, where they were maintained on a normal mouse chow diet in a 12/12-h light/dark cycle. All animal procedures were approved by the Institutional Animal Care and Use Committee (IACUC) of the University of California, Irvine, and were conducted in accordance with the Association for Research in Vision and Ophthalmology Statement for the Use of Animals in Ophthalmic and Visual Research.

### Cell Line Generation

NIH3T3-RPE65 (wt) and NIH3T3-RPE65 (rd12) stable cell lines were generated by transduction of NIH3T3 cells with retrovirus obtained from Phoenix-Eco cells transfected with either pMXs- RPE65(wt)-IRES-GFP or pMXs-RPE65(rd12)-IRES-GFP according to a previously published protocol. Transduced cells were sorted by a FACSAria cell sorter (BD Biosciences) to selectively collect transduced cells and ensure comparable EGFP expression between NIH3T3- RPE65 (wt) and NIH3T3-RPE65 (rd12). To make RPE65 expression vectors, wildtype or rd12 mouse Rpe65 cDNA sequence, flanked by EcoRI and NotI, were purchased from Gene Universal, and cloned into the multiple cloning site of pMXs-IRES-GFP (a gift from Dr. T. Kitamura at the University of Tokyo). The downstream sequence of the internal ribosomal entry site (IRES) and enhanced green fluorescence protein (EGFP) allows co-expression of RPE65 and EGFP, thereby enabling cell sorting by flow cytometry. Cells were maintained in growth medium (GM) composed of Dulbecco’s modified Eagle’s medium (Gibco) supplemented with 10% (v/v) heat-inactivated fetal bovine serum and 1% (v/v) Penicillin/Streptomycin mix (100 units/mL penicillin and 100 units/mL streptomycin). Cells were maintained at 37° C. in 5% CO₂.

### In Vitro HDR Validation

Each sgRNA sequence targeting the rd12 mutation was cloned into pX330-U6-Chimeric_BB- CBh-hSpCas9 (a gift from Feng Zhang, Addgene plasmid #42230). (the final cloned product referred to as pX330-sgRNA). 140-nt single-stranded donor template was synthesized by Integrated DNA Technologies (IDT). To confirm the DNA targeting by sgRNA, each well in a 6-well plate of rd12 cells was transfected with 2 µg of pX330- sgRNA using Lipofectamine 2000 (Thermo Fisher) following the manufacturer’s protocol. At 48 h post-transfection, SURVEYOR nuclease assay (IDT) was performed. To evaluate the HDR efficiency in vitro, 1x10⁶ rd12 cells were nucleofected with 6 µg of pX330-sgRNA and 120 pmol of donor template with Program A-024 of the Amaxa Cell Line Nucleofector Kit R (Lonza). Cells were collected for deep targeted DNA sequencing 96 h after nucleofection.

### In Vivo HDR Analysis

For Cas9 delivery, pAAV-EFS-SpCas9 (a gift from Ryohei Yasuda, Addgene plasmid #104588) was used. For sgRNA and donor delivery, pAAV-donor-U6-sgRNA1 was synthesized by cloning. Both plasmids were packaged into AAV type 1 capsid by Penn Vector Core.

### Construction of ABE and sgRNA Expression Plasmids for in Vitro Base Editing Validation

pCMV-ABEmax (a gift from David Liu, Addgene plasmid #112095) and xCas9(3.7)-ABE(7.10) (a gift from David Liu, Addgene plasmid #108382) were used to express ABE and xABE, respectively. For U6-sgRNA expression plasmids, five different gRNA oligonucleotides were synthesized (Genewiz) and cloned into the pSPgRNA vector (a gift from Charles Gersbach, Addgene #47108) using the BbsI restriction site.

### Virus Production for in Vivo ABE Delivery

To generate a single lentiviral construct co-expressing sgRNA and ABE, the U6-sgRNA cassette was PCR amplified with the primers including restriction sites, MluI and BcuI, and cloned into the compatible restriction sites in pCMV-ABEmax plasmid. The resulting U6-sgRNA- CMV-ABEmax plasmid was subsequently cloned into the third generation lentiviral transfer vector, LentiCRISPRv2GFP (a gift from David Feldser, Addgene #82416), replacing the sequences between the 5′ and 3′ long terminal repeat (LTR) sequences. The final cloned plasmids were packaged into lentivirus particles by Signagen. AAV1-CMV-GFP (Addgene viral prep #105545-AAV1), serving as a fluorescence marker, was purchased from Addgene.

### Plasmid Transfection for in Vitro Validation

NIH3T3-RPE65 (rd12) cells were seeded on a 24-well plate 18 h prior to transfection. At ~70% confluency, cells were transfected with 750 ng of ABEmax or xABE plasmid and 250 ng of sgRNA plasmid using 1.5 µl of Lipofectamine 3000 (Thermo Fisher) per well.

### Off-Target Analysis Using CIRCLE-Seq

CIRCLE-seq was performed as previously described. Genomic DNA from a C57/BL6 mouse was isolated from liver tissue using a Gentra Puregene Tissue Kit (Qiagen). PCR amplification before sequencing was conducted using PhusionU polymerase, and products were gel-purified and quantified with a Qubit High-sensitivity kit before loading onto an Illumina MiSeq. Data were processed using the CIRCLE-Seq analysis pipeline with parameters: “Read_threshold: 4; window_size: 3; mapq_threshold: 50; start_threshold: 1; gap_threshold: 3; mismatch_threshold: 6; mIllued_analysis: True”.

### Deep Targeted Sequencing Analysis

Genomic DNA from cultured cells or mouse RPE tissue was isolated using the DNeasy Blood and Tissue Kit according to the manufacturer’s instructions. Following DNA isolation, 265 - 308 bp PCR amplicons of on- and off-target predicted sites for Rpe65 were generated using primers with partial Illumina adapter sequences and then purified using the QIAquick PCR Purification Kit (Qiagen). Samples were sequenced on an Illumina Miseq by Genewiz. Between 70,000 and 100,000 NGS reads for each sample were generated on paired-end 2 x 250 bp run.

### Mouse Subretinal Injection

Mice were anesthetized by intraperitoneal injection of a cocktail consisting of 20 mg/ml ketamine and 1.75 mg/ml xylazine in phosphate-buffered saline at a dose of 0.1-0.13 ml per 25 g body weight, and their pupils were dilated with topical administration of 1% tropicamide ophthalmic solution (Akom). Subretinal injections were performed using an ophthalmic surgical microscope (Zeiss). An incision was made through the cornea adjacent to the limbus at the nasal side using a 26-gauge needle. A 35-gauge blunt-end needle (World Precision Instruments) connected to an RPE-KIT (World Precision Instruments) by SilFlex tubing was inserted through the corneal incision while avoiding the lens and pushed through the retina. Each mouse received 1 µl of injection compound per eye.

### HPLC Retinoid Profiling in Mouse Eye

Mice were dark-adapted for 2 days prior to the enucleation of the eyes. The retinoid analysis of light-exposed mouse eyes included a 0.5 s flash exposure from a 30 cm distance prior to eye enucleation. Two eyes from WT, untreated and treated rd12 mice were homogenized in 10 mM sodium phosphate buffer, pH 8.0, containing 50% methanol (v/v) and 100 mM hydroxylamine. After 15 min of incubation at room temperature, 2 ml of 3 M sodium chloride was added. The resulting sample was extracted twice with 3 ml of ethyl acetate. Then, the combined organic phase was dried in vacuo and reconstituted in 300 µl of hexane. Extracted retinoids (100 µl) were separated on a normal phase HPLC column (Sil; 5 µm, 4.6 × 250 mm; Agilent Technologies) equilibrated with a stepwise gradient of 0.6% ethyl acetate in hexane at an isocratic flow rate of 1.4 ml/min for 17 min and 10% ethyl acetate in hexane at an isocratic flow rate of 1.4 ml/min for 25 min. Retinoids were detected by monitoring their absorbance at 325 nm.

### Western Blot Analysis

To prepare the protein lysate from transfected cells, each well of cells was lysed in 100 µl ice- cold RIPA buffer (Cell Signaling Technology) with protease inhibitors (Sigma-Aldrich) by maintaining constant agitation for 30 min at 4° C. The lysates were centrifuged for 30 min at 20,000 x g at 4° C., and the supernatant was saved for gel loading. To prepare the protein lysate from the mouse RPE tissue, the dissected mouse eyecup, consisting of RPE, choroid and sclera, was transferred to a microcentrifuge tube containing 30 µl of RIPA buffer with protease inhibitors, and homogenized with a motor tissue grinder (Fisher Scientific) and centrifuged for 30 min at 20,000 x g at 4° C. The resulting supernatant was pre-cleared with Dynabeads Protein G (Thermo Fisher) to remove contaminants from blood prior to gel loading. The lysates were mixed with NuPAGE LDS Sample Buffer and NuPAGE Sample Reducing Agent and incubated at 70° C. for 10 min, and separated using a NuPAGE 4-12% Bis-Tris gel and transferred onto PVDF membrane (Invitrogen), followed by 1 h blocking in 5% (w/v) non-fat milk in PBS containing 0.1% (v/v) Tween 20 (PBS-T). The membrane was incubated with primary antibody diluted in 1% (w/v) non-fat milk in PBS-T overnight at 4° C. Primary antibodies include mouse anti-RPE65 monoclonal antibody (1:1,000; in-house production); mouse anti-Cas9 monoclonal antibody (1:1,000; Invitrogen); rabbit anti-β-actin polyclonal antibody (1:1,000; Cell Signaling Technology); rabbit anti-α-tubulin polyclonal antibody (1:1,000; Cell Signaling Technology). After overnight incubation, membranes were washed three times with PBS-T for 5 min each and then incubated with secondary antibody for 1 h at room temperature. Secondary antibodies include goat anti-mouse IgG-HRP antibody (1:5,000; Cell Signaling Technology) and goat anti-rabbit IgG-HRP antibody (1:5,000; Cell Signaling Technology). After washing the membrane three times with PBS-T for 5 min each, protein bands were visualized after exposure to SuperSignal West Pico Chemiluminescent substrate (Thermo Fisher).

### Immunocytochemistry

Cells were fixed, permeabilized, and blocked using an Image-iT Fixation/Permeabilization Kit following the manufacturer’s protocol (Thermo Fisher). Cells were immunostained with mouse anti-RPE65 monoclonal antibody (1:1,000) and rabbit anti-GRP78 BiP polyclonal antibody (1:1,000; Abcam) diluted in PBS-T followed by the corresponding Alexa Fluor 555 goat anti- mouse IgG (1:1,000; Thermo Fisher), Alexa Fluor 647 goat anti-rabbit IgG (1:1,000; Thermo Fisher) and DAPI. Samples were mounted in ProLong Gold antifade reagent (Invitrogen) and imaged with a Keyence BZ-X810 All-in-One Fluorescence microscope (Keyence).

### Immunohistochemistry of RPE Flatmounts and Cryosections

Mouse eyes were fixed with 4% paraformaldehyde in PBS (Santa Cruz Biotechnology) for 20 min at room temperature and washed three times in PBS for 5 min each. To make RPE flatmounts (RFM), the anterior segment and retina were removed from the posterior eyecup under a dissecting microscope, and four radial cuts were made toward the optic nerve to flatten the eyecup into RFM. To make retinal cryosections, fixed eyes were dehydrated with 30% sucrose in PBS, embedded in O.C.T. (Sakura), and then flash-frozen for cryosectioning at 10 µm thickness. The following procedures are applicable for both RFMs or cryosections. Samples were permeabilized in 0.5% Triton-X in PBS for 30 min, blocked in 3% BSA in PBS for 30 min and incubated with appropriate primary antibody, including mouse anti-RPE65 antibody (1:100) and rabbit anti-ZO-1 polyclonal antibody (1:100; Invitrogen; 61-7300), overnight at 4° C. Next day, samples were washed three times in PBS for 5 min each and then incubated with secondary antibody, Alexa Fluor 594-conjugated goat anti-mouse IgG (1:200; Thermo Fisher) and Alexa Fluor 647-conjugated goat anti-rabbit IgG (1:200; Thermo Fisher), for 2 h at room temperature in the dark. Samples were incubated in 1 µg/mL DAPI (Thermo Fisher) in PBS for 10 min and washed three times in PBS for 5 min each. Samples were mounted with ProLong Gold mounting media and imaged as described above.

### Electroretinography

Prior to recording, mice were dark adapted for 24 h. Under a safety light, mice were anesthetized by intraperitoneal injection of a cocktail consisting of 20 mg/ml ketamine and 1.75 mg/ml xylazine in phosphate-buffered saline at a dose of 0.1-0.13 ml per 25 g body weight.

Pupils were dilated with 1% tropicamide (Henry Schein), and then 2.5% hypromellose was applied to keep the corneas hydrated and to facilitate electrical conductivity. Active recording electrodes were placed onto the corneas, and reference and ground electrodes were positioned subdermally between the ears and on the tail, respectively. The eyes were stimulated with a green light (peak emission 544 nm, bandwidth ~ 160 nm) stimulus at of -0.3 log (cd· s/m²). The responses for 10 stimuli with an inter-stimulus interval of 10 s were averaged together, and the a- and b-wave responses were acquired from the averaged ERG waveform. A-wave is the first negative polarity deflection after stimulus onset, and b-wave is the first positive peak occurring after a-wave trough. The ERGs were recorded with the Celeris rodent electrophysiology system (Diagnosys LLC) and analyzed with Espion V6 software (Diagnosys LLC).

### Optomotor Response Test

Optomotor responses were assessed using a commercial optomotor response (OMR) platform that utilizes automated head tracking and behavior analysis (Phenosys). The software automatically compares horizontal head movement in relation to the speed of a moving vertical grating stimulus and quantifies correct/incorrect tracking behavior. The OMR arena was dimmed by using neutral density filters in front of the stimulus displays. The ambient luminance was measured at ~ 1 lux corresponding to mesopic, roughly twilight light level. When a light-adapted mouse was placed on the OMR arena’s elevated platform, rotating (12 °/s) vertical sinusoidal grating stimuli were presented for 10 min per trial. The spatial frequency of the grating was set at 0.1 cycles per degree (CPD) of visual angle. This stimulus was presented at differing contrast between the light and dark; 5, 7.5, 10, 12.5, 15, 17.5, 20, 25, 50 and 100% contrast. Stimulation at each contrast level lasted 60 s, and contrast levels were presented in a randomized order, except that each session was always started with 100% contrast stimuli, which was omitted from the analysis due to mouse acclimatization. Each mouse was tested in at least four trials (max 2 trials per day), and the first 10-min trial was considered acclimatization and not used in the analysis. The performances of the remaining rest trials were averaged for analysis, excluding those 60 s stimulus periods that led to a correct/incorrect ratio smaller than 0.8. Six untreated rd12 mice and five treated mice were tested, as well as six age-matched C57BL/6J mice to show normal pigmented mouse performance.

### Primary Visual Cortex (V1) Electrophysiology

Mice were initially anesthetized with 2% isoflurane in a mixture of N₂O/O₂ (70%/30%) then placed into a stereotaxic apparatus. A small, custom-made plastic chamber was glued to the exposed skull. One day after recovery, re-anesthetized animals were placed in a custom-made hammock, maintained under isoflurane anesthesia (1-2% in a mixture of N₂O/O₂) and a single tungsten electrode was inserted into a small craniotomy above the visual cortex. Once the electrode was inserted, the chamber was filled with sterile agar and sealed with sterile bone wax.

During recording sessions, animals were sedated with chlorprothixene hydrochloride (1 mg/kg; IM) and kept under light isoflurane anesthesia (0.2 - 0.4%). EEG and EKG were monitored throughout the experiments and body temperature was maintained with a heating pad (Harvard Apparatus).

Data was acquired using a 32-channel Scout recording system (Ripple). The local field potential (LFP) from multiple locations was band-pass filtered from 0.1 Hz to 250 Hz and stored together with spiking data on a computer with 1 kHz sampling rate. The LFP signal was cut according to stimulus time stamps and averaged across trials for each recording location to calculate visually evoked potentials (VEP). The spike signal was band-pass filtered from 500 Hz to 7 kHz and stored in a computer hard drive at 30 kHz sampling frequency. Spikes were sorted online in Trellis (Ripple) while performing visual stimulation. Visual stimuli were generated in Matlab (Mathworks) using Psychophysics Toolbox and displayed on a gamma-corrected LCD monitor⁵² (55 inches, 60 Hz; 1920 x 1080 pixels; 52 cd/m² mean luminance). Stimulus onset times were corrected for LCD monitor delay using a photodiode and microcontroller (in-house design).

For recordings of visually evoked single-cell responses, the eyes were first stimulated with 100 repetitions of a 500 ms bright flash stimulus (105 cd/m2). Receptive fields for visually responsive cells were then located using square-wave drifting gratings, after which optimal orientation/direction, spatial and temporal frequencies were determined using sine wave gratings. Spatial frequencies tested ranged between 0.001 - 0.5 cycles/degree of visual angle. Temporal frequencies tested were 0.1 to 10 cycles/s. With these optimal parameters, size tuning was assessed using apertures of 1 - 110° at 100% contrast. With the optimal size, temporal and spatial frequencies, and at high contrast, the orientation selectivity of the cell was tested again using 16 directions at 22.5° increments. This was followed by testing contrast.

### Analysis of V1 Electrophysiology

The LFP signal was normalized using z-score standardization. The response amplitude of LFP was calculated as a difference between the peak of the positive and negative components in the VEP waveform. The response latency was defined as the time point where maximum response occurred. The maximum of the response was defined as maximum of either the negative or positive peak.

Tuning curves were calculated based on average spike rate. Optimal visual parameters were chosen as the maximum response value. Orientation selectivity index (OSI) was calculated as follows:

$$O S I = \frac{\left|{\sum }_{n} {R}_{n} e x p \left(i {θ}_{n}\right)\right|}{\left({\sum }_{n} \left|{R}_{n}\right|\right)} ,$$

where θₙ is the nth orientation of the stimulus and Rₙ is the corresponding response.

The orientation tuning bandwidth was measured in degrees as the half-width at half-height (HWHH; 1.18 x σ) based on fits to Gaussian distributions using:

$${R}_{{O}_{S}} = b a s e l i n e + {R}_{p} {e}^{\frac{{\left({O}_{s} - {O}_{p}\right)}^{2}}{2 {σ}^{2}}} + {R}_{n} {e}^{\frac{{\left({O}_{s} - {O}_{p} + 180\right)}^{2}}{2 {σ}^{2}}} ,$$

where Oₛ is the stimulus orientation, R_{Os} is the response to different orientations, Oₚ is the preferred orientation, Rₚ and Rₙ are the responses at the preferred and non-preferred direction, σ is the tuning width, and ‘baseline’ is the offset of the Gaussian distribution. Gaussian fits were estimated without subtracting spontaneous activity, similar to the procedures of Alitto and Usrey.

Size tuning curves were fitted by a difference of Gaussian (DoG) function:

$${R}_{s} = {K}_{e} {\int }_{- s}^{s} {e}^{{\left(- \frac{x}{{r}_{e}}\right)}^{2}} d x - {K}_{i} {\int }_{- s}^{s} {e}^{{\left(- x / {r}_{i}\right)}^{2}} d x + {R}_{0} ,$$

in which Rₛ is the response evoked by different aperture sizes. The free parameters, Kₑ and re, describe the strength and the size of the excitatory space, respectively; Ki and ri represent the strength and the size of the inhibitory space, respectively; and R₀ is the spontaneous activity of the cell.

The suppression index (SI) was calculated from fitted tuning curves using the following equation:

$$S l = \frac{{R}_{o p t} - {R}_{s u p p}}{{R}_{o p t}} ,$$

where Rₒₚₜ indicates response at preferred size, and Rₛᵤₚₚ indicates response at suppressive surround stimulus size.

The optimal spatial and temporal frequency was extracted from the data fitted to Gaussian distributions using the following equation:

$${R}_{S F / T F} = b a s e l i n e + {R}_{p r e f} {e}^{- \frac{{\left(S F / T F - S F / T {F}_{p r e f}\right)}^{2}}{2 {σ}^{2}}} ,$$

where R_{SF/TF} is the estimated response, R_{pref} indicates response at preferred spatial or temporal frequency, SF/TF indicates spatial or temporal frequency, σ is the standard deviation of the Gaussian, and baseline is the Gaussian offset.

The contrast tuning was fitted by using the Naka-Rushton equation:

$$R \left(C\right) = \frac{g {C}^{n}}{{C}_{50}^{n} + {C}^{n}} ,$$

where g is the gain (response), C₅₀ is a contrast at mid response, and n is the exponent. For the contrast tuning fit the background activity was subtracted from the response curve and values below background standard deviation were changed to 0.

Average differences between animal groups were considered statistically significant at P ≤ 0.05 for two-tailed Mann-Whitney U-tests. Mean values given in the results include error bars for the standard error of the mean (SEM). All offline data analysis and statistics were performed in Matlab (Mathworks).

### Results

We corrected a de novo nonsense mutation in the Rpe65 gene on exon 3 (c.130 C>T; p.R44X) in the rd12 mouse model (FIG. 1a). A homologous mutation has recently been identified as an LCA-causing mutation among the Chinese population, highlighting the translational relevance of the animal model. The rd12 mutation in mouse abolishes the expression of RPE65, a key isomerase in the classical visual cycle that regenerates active visual chromophore, 11-cis-retinal. Therefore, these mice display visual cycle blockade and profoundly impaired visual function. Fortuitously, the production of 11-cis-retinal offers a direct biochemical readout for the phenotypic improvement of gene repair, making the rd12 mouse model a robust system to test genome-editing approaches.

We show that subretinal delivery of ABE corrects the pathogenic mutation by converting A to G on the complementary strand of the Rpe65 gene with precision and minimal undesired mutations. In previous reports that employed a base editor in other disease models, the target mutation had the “NGG” PAM sequence, whereas the rd12 mouse model does not contain an NGG PAM sequence properly positioned to correct the mutation. To overcome this obstacle, we first tested xCas9-3.7-ABE (xABE), an evolved ABE that can recognize a broad range of PAMs, with five different protospacer sequences placing the target “A” within the activity window of 4 to 8 (referred to as gRNA-A4 to gRNA-A8) (FIGS. 1b, d).

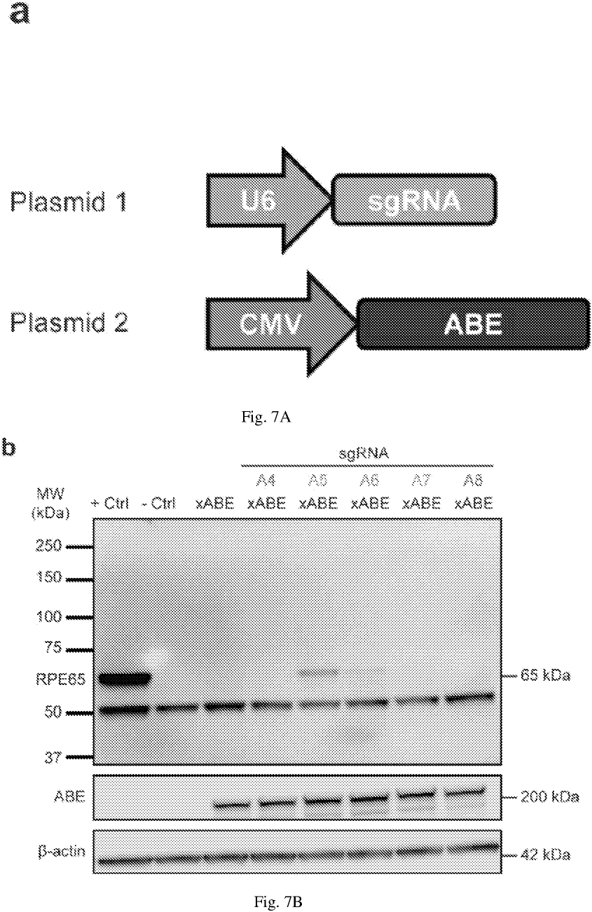

To test base editing efficacy in vitro, we generated a reporter NIH3T3 cell line by stably integrating rd12 mutant Rpe65 cDNA, hereby referred to as the rd12 cell line (FIG. 1c). We transfected rd12 cells with xABE and one of each sgRNA expression plasmids, and subsequently determined RPE65 expression by Western blot analysis 48 h after transfection (FIG. 7a). Cells transfected with gRNA-A5 and gRNA-A6 each showed an RPE65 band at the expected molecular weight, although A5 had a more intense band than A6 (FIG. 7b).

Next, we repeated the transfection by replacing xABE with wt codon-optimized ABE (ABE) to test if the mutation without a canonical NGG PAM sequence can be targeted with ABE. Co-transfection with ABE resulted in a higher amount of RPE65 rescue with gRNA-A5.

(NAG PAM) and gRNA-A6 (NGA PAM) than with xABE (FIGS. 1E, F). We reasoned that codon- optimization of ABE yields a stable and higher protein expression, leading to more frequent base editing activity even at sites lacking a canonical NGG PAM.

Deep sequencing analysis on A5- and A6-treated cells showed a correction rate of 2.88% ± 0.19% and 3.43% ± 0.04% respectively (FIG. 1g). We reasoned that the low correction frequency is likely attributed to two factors, beyond the use of non-canonical PAMs. The integration of multiple copies of viral Rpe65^{rd12} cDNA in each cell may leave some editable sites untargeted by ABE. Secondly, a low transfection efficiency (<50%) of the rd12 cell line, as demonstrated by transfection with a mCherry reporter plasmid, further reduces the probability of co-transfection in each cell (data not shown). Nevertheless, the base editing of the target mutation (T₇>C) was the most frequent alteration in both A5 and A6 transfected cells. Besides the target base editing, two other allelic variants were observed. In A5, base editing occurred at an adenine located two nucleotides away from the target base (T₅>C) at 2.55% ± 0.17%, and the conversion of both adjacent and the target adenine (T₇>C and T₅>C) was observed at 1.07% ± 0.01%. A6, on the other hand, showed very low frequency of these two variants at 0.19% ± 0.02% and 0.14% ± 0.01%, respectively. Besides these conversions, no other DNA modification, including insertions, deletions and substitutions, was observed above the background level in the non-transfected control group. The sequencing results from in vitro experiments confirmed that ABE can correct the nonsense mutation with sgRNA-A5 or -A6 while minimizing indel mutations.

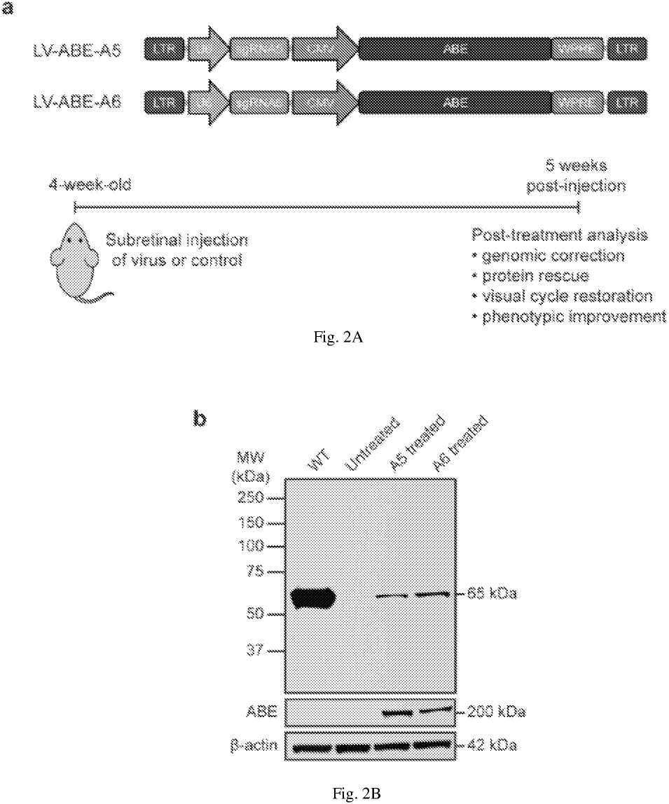

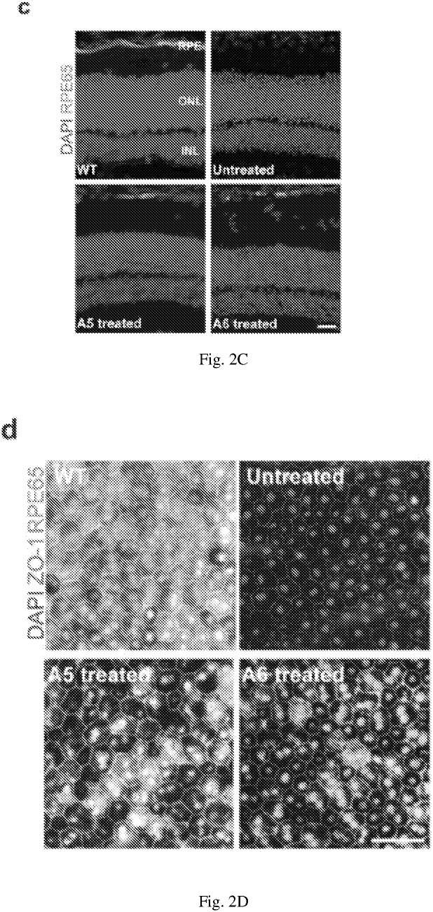

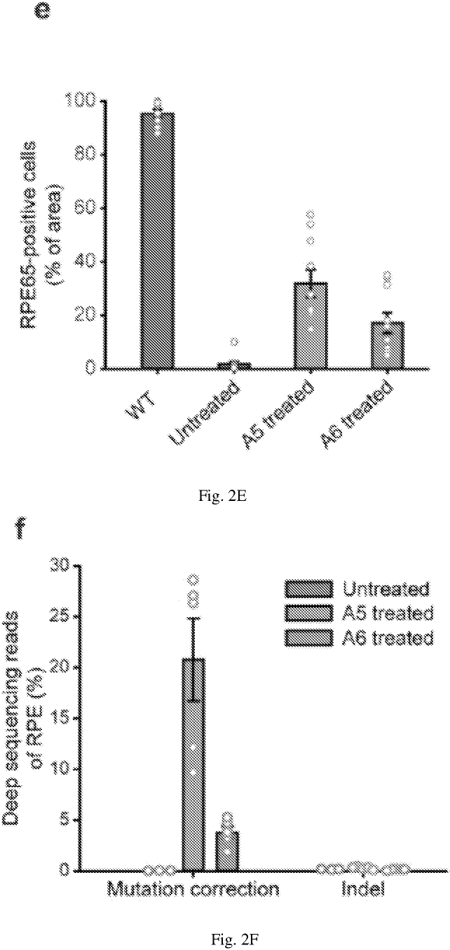

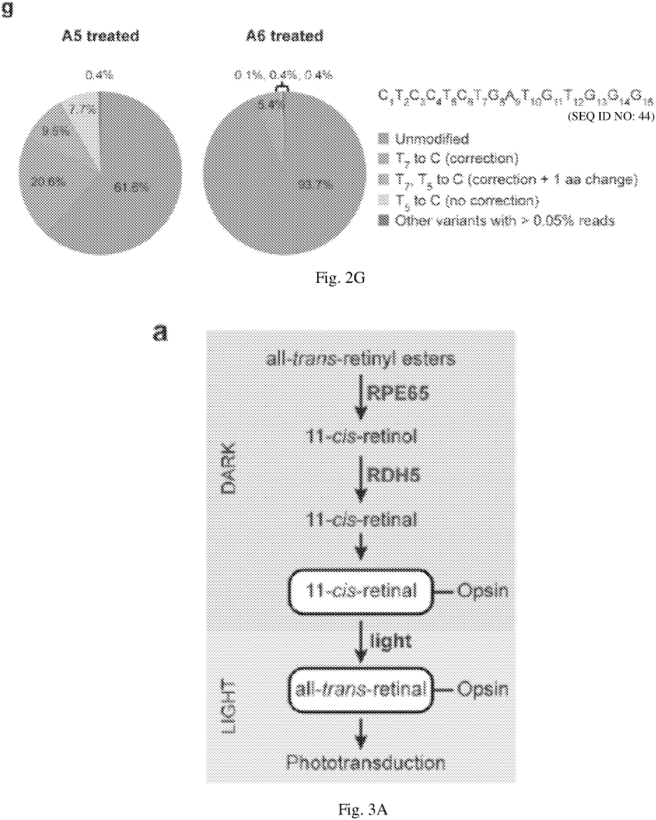

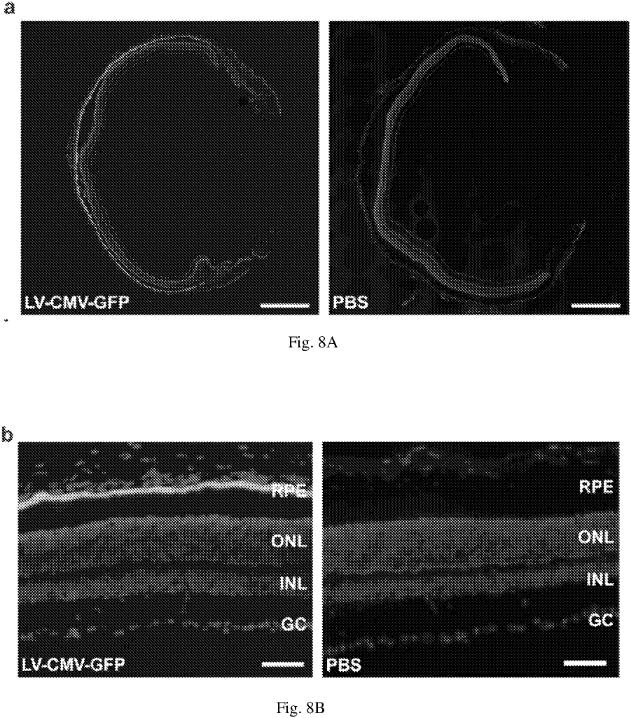

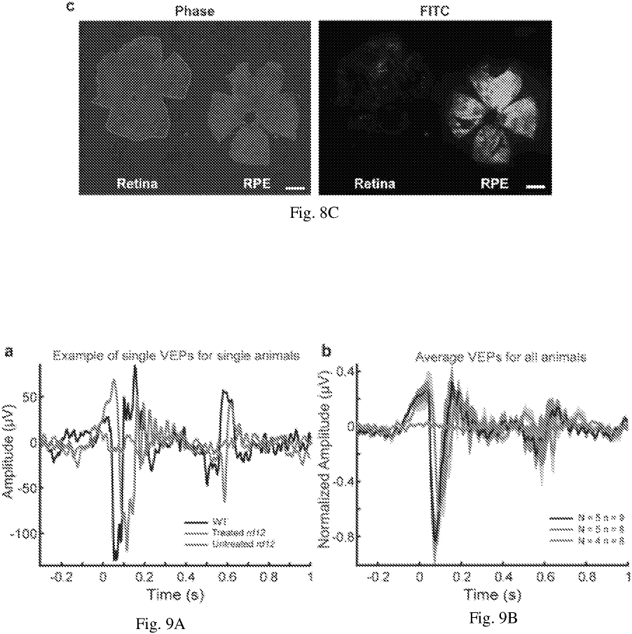

To deliver the sgRNA and ABE to mouse RPE cells in vivo, we generated two lentiviruses (LV) encoding ABE and either sgRNA A5 or sgRNA A6 (referred to as LV-ABE-A5 and LV-ABE-A6) (FIG. 2a). We chose LV over recently described AAV delivery vectors to maximize transduction and ABE expression to evaluate the greatest possible efficacy of this approach. LV also exhibits RPE-specific tropism when injected subretinally to the mouse eye, obviating the need for a tissue specific promoter (FIGS. 8A-C).

We treated rd12 mice by subretinal injection at 4 weeks of age with either LV-ABE-A5 (1X10⁶ transducing units (TU) per eye), LV-ABE-A6 (1X10⁶ TU per eye), or PBS (control). In each group, we co-injected AAV1-CMV-GFP (5X10⁷ genome copies (GC) per eye) to ensure the successful delivery by measuring fundus green fluorescent protein (GFP) fluorescence with an in vivo scanning laser ophthalmoscope (SLO) 2 weeks after the injection, and the eyes which had >70% GFP fluorescence were used for post-treatment analysis. Five weeks after the injection, we first evaluated RPE65 protein restoration in the treated eye, which provides a rough validation for mutation correction. The Western blot analysis of RPE extracts obtained from mice treated with LV-ABE-A5 or LV-ABE-A6 both showed the RPE65 band, although not as intense as the band from WT control (FIG. 2B). The correct localization of rescued RPE65 was confirmed by immunohistochemistry (FIG. 2C). To assess the approximate percentage of corrected cells from each eye, RPE tissue was processed as a whole mount and analyzed by immunofluorescence, which showed a rescue of 31 % in A5 and 20 % in A6 treatment groups (FIGS. 2D, E).

To quantify the correction efficiency by ABE, we amplified the region around the rd12 mutation by PCR from the DNA obtained from RPE and performed deep amplicon sequencing. We detected mutation correction (T₇>C) at the rate of 20.8 ± 4.1% in the A5 and 3.8 ± 0.6% in A6 treated RPE tissue, respectively. We note that these numbers are slightly underestimated, because the DNA samples from the RPE cells included cells from choroid and sclera, which were not exposed to ABE, from dissections. In contrast, we observed no substantial indel mutation with the rate of 0.29 ± 0.05% in A5 and 0.14 ± 0.03% in A6 (0.16 ± 0.03% in control) (n = 5 each group, FIG. 2f). To further examine other potential base substitutions by ABE, we analyzed the composition of allelic variants in a representative RPE sample from each treatment group. In both A5 and A6, a single base editing at the target mutation (T₇>C) was the most frequent allele (FIG. 2g). Two base editing (T₇>C and Ts>C) and a single non-target base editing (Ts>C) were the second and third most frequent alleles in the A5 sample, and the opposite in A6.

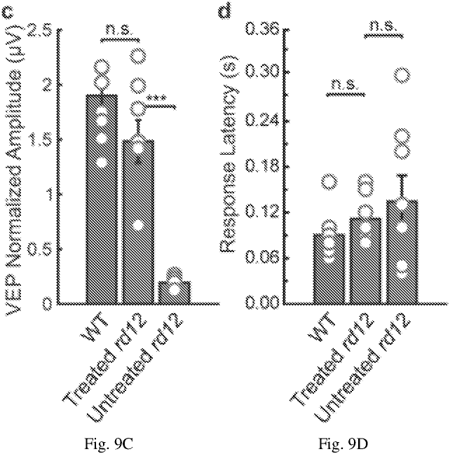

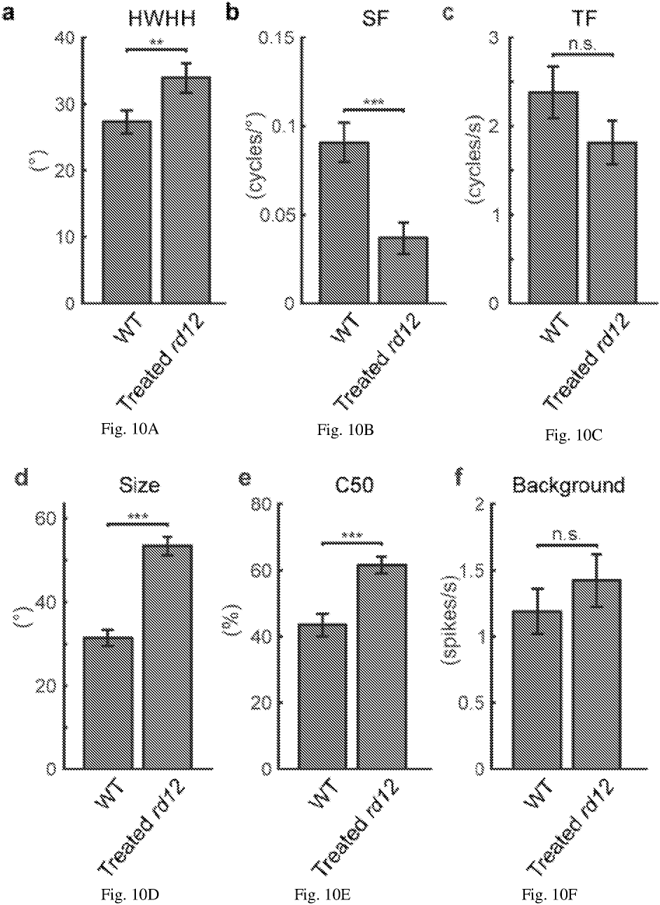

We also assessed off target activity of both A5 and A6 in the RPE tissue by examining the top ten potential off-target sites identified by unbiased, genome-wide CIRCLE- seq. In each treatment group, we sequenced ten off-target sites in treated RPE tissue and did not detect off-target editing above the background level in the untreated RPE tissue (FIGS. 9 and 10a-d) corresponding to the findings of previous reports showing a low off-target activity of ABE. While A5 and A6 both demonstrated undetectable off-target activity in the rd12 mice, A5 showed a higher on-target base editing efficiency, for which we decided to further evaluate the improvements in the disease phenotype of rd12 mice treated with LV-ABE-A5. From here on, all post-treatment evaluation was done in rd12 mice injected with LV-ABE-A5.

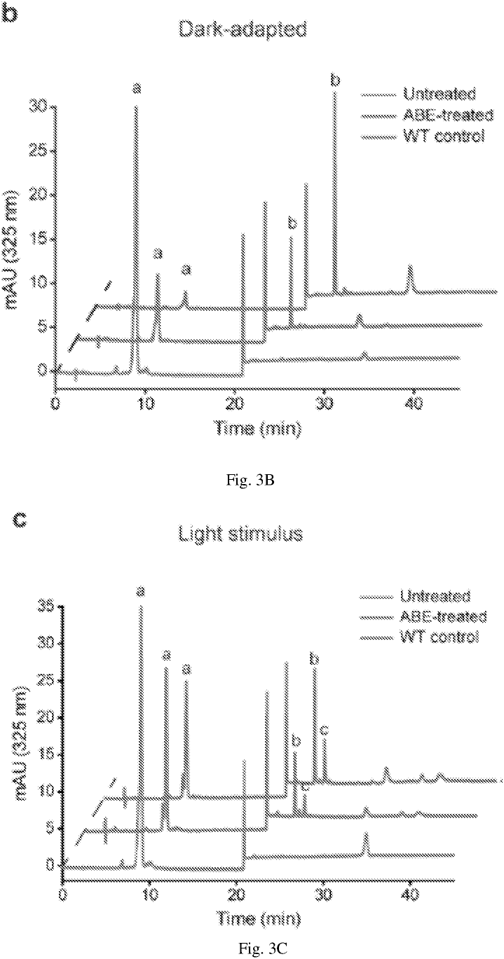

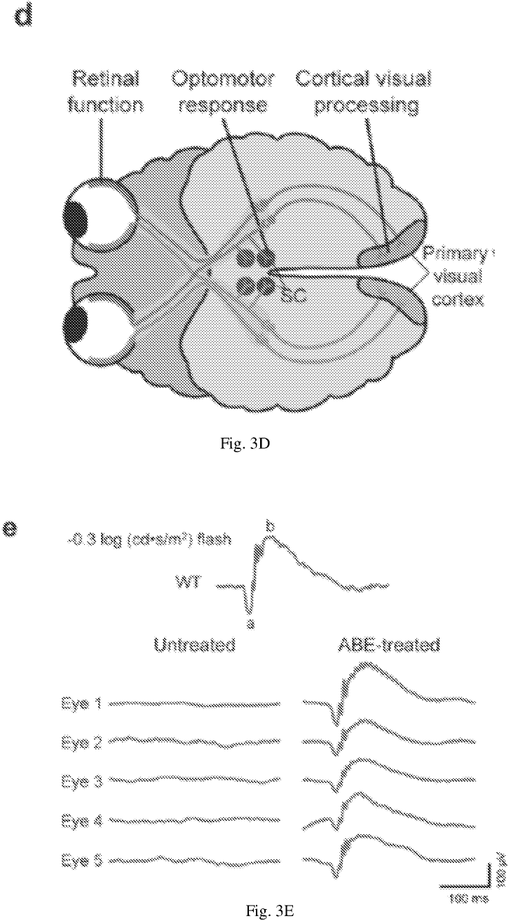

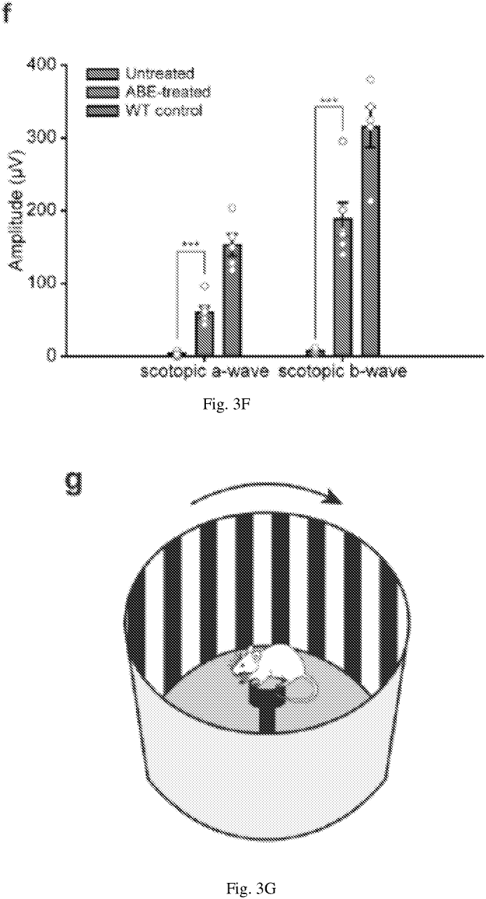

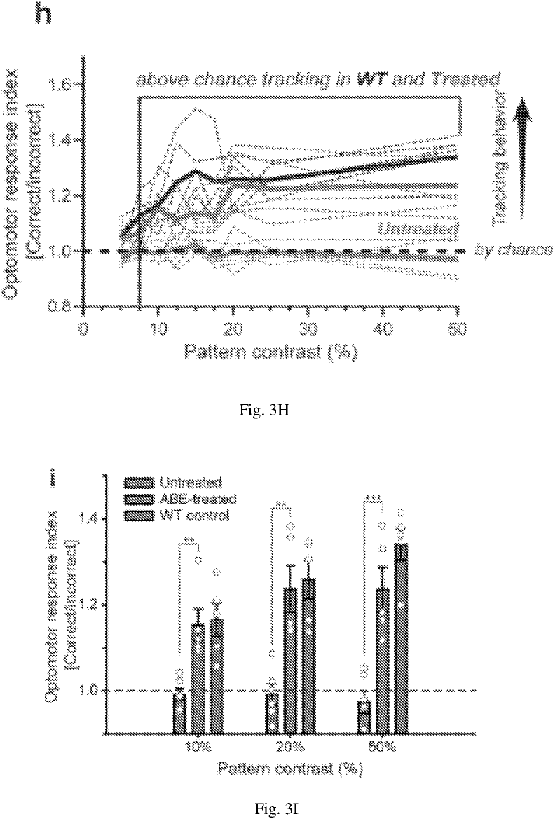

First, we evaluated whether a functional visual cycle is restored in the ABE-treated rd12 mice. In a classical visual cycle, RPE65 isomerizes all-trans-retinyl esters into 11-cis-retinol, which is an essential process for regeneration of the active visual chromophore, 11-cis-retinal (FIG. 3A). In rd12 mice, this reaction is blocked due to the absence of RPE65, resulting in extreme 11-cis-retinal deficiency and accumulation of all-trans-retinyl esters. In retinoid analysis by high performance liquid chromatography (HPLC), the ABE-treated rd12 mouse eyes revealed substantial production of 11-cis-retinal and a reduction of all-trans-retinyl esters, indicating a restoration of the visual cycle (FIG. 3B). Furthermore, we confirmed that the new supply of 11-cis-retinal can photoisomerize to all-trans-retinal immediately following a flash stimulus (FIG. 3C).

Next, we determined whether a recovery of visual chromophore regeneration could restore the function of different cell types comprising the primary visual pathways in treated mice (FIG. 3D). First, we assessed retinal cell activity by scotopic electroretinography (ERG). This technique allows functional assessment of photoreceptors and downstream retinal interneurons through information encoded in the a-wave and b-wave, respectively. The untreated rd12 mice exhibited a complete loss of amplitude in both a-wave and b-wave in response to an intermittent flash stimulus of -0.3 log (cd•s/m²) intensity, whereas ABE-treated mice recovered a-wave and b-wave amplitudes of 39% and 60% of the wt control responses (FIGS. 3E, F).

Next, we assessed the mice optomotor responses mediated by the superior colliculus (SC), which is the most prominent retinal target in the mouse. In mice, more than 70% of retinal axons project to the superficial layers of the SC, and the remaining 30% project to the primary visual cortex (V1) via the lateral geniculate nucleus. Therefore, the optomotor response test provides a robust means of evaluating the functional integrity of the visual pathway in mice. The quantitative optomotor response test (qOMR) system measures visual function by quantifying the animal’s reflexive head movements to rotating stripes (FIG. 3G). In moderate ambient luminance of ~1 lux (i.e., low twilight light level), both WT and treated rd12 mice showed significant tracking response starting from 7.5% contrast between the white and black sinusoidal gratings (FIG. 3H, I). In contrast, untreated rd12 mice did not show tracking behavior even for the highest contrast stimuli of 50%.

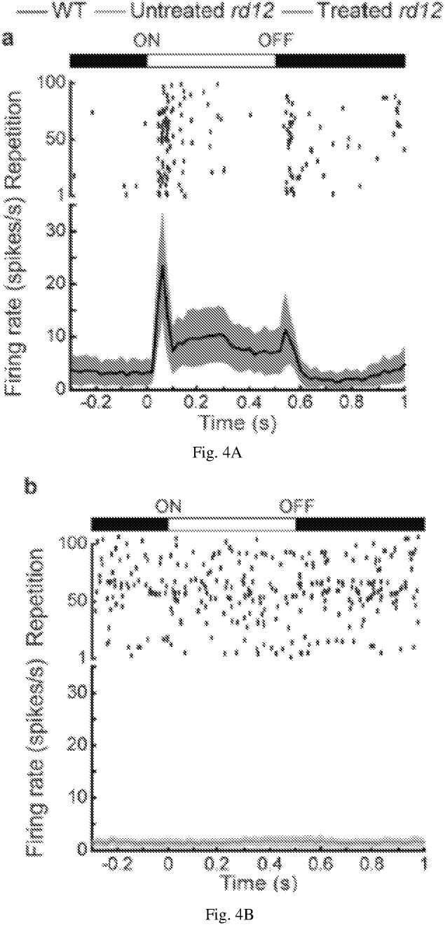

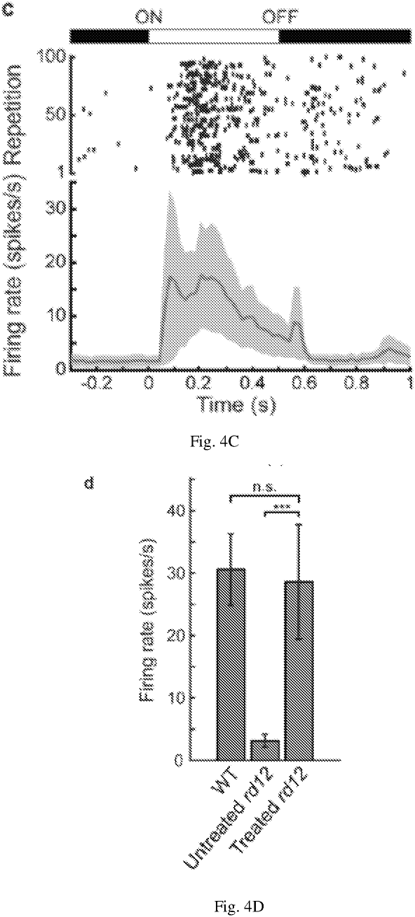

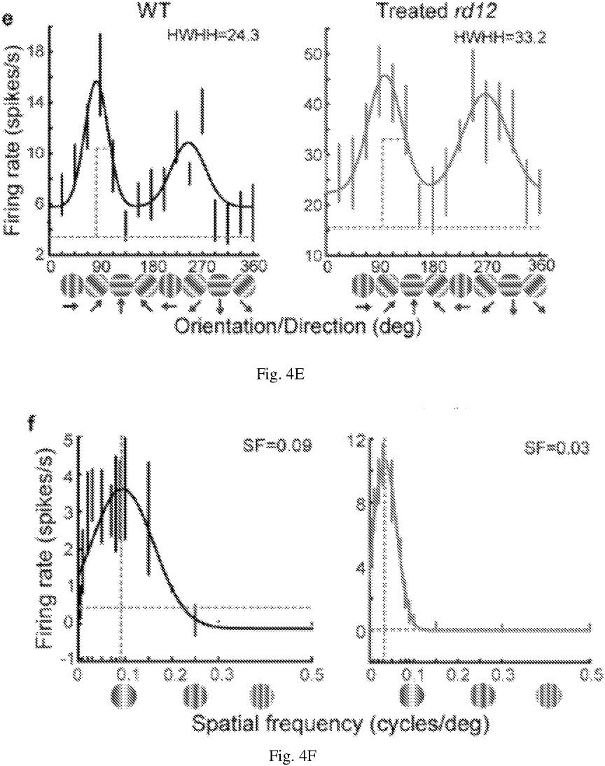

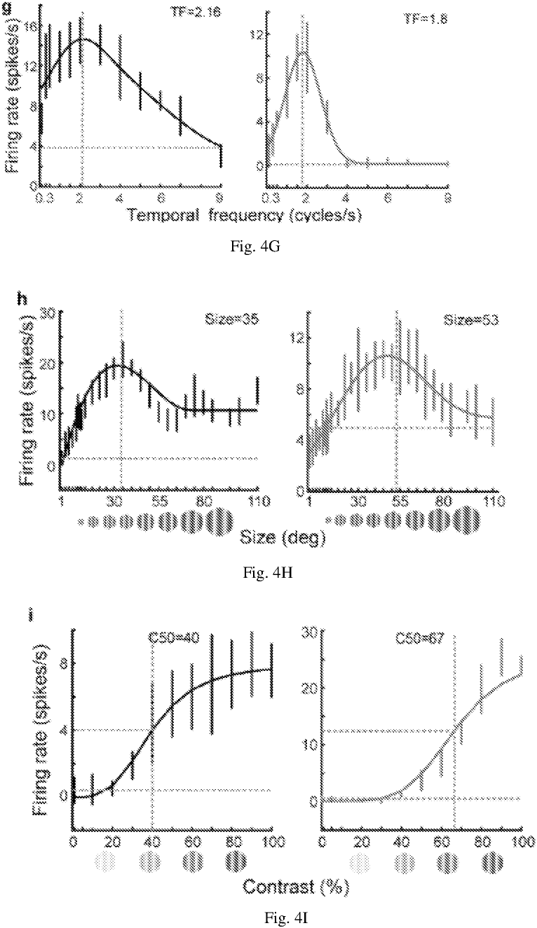

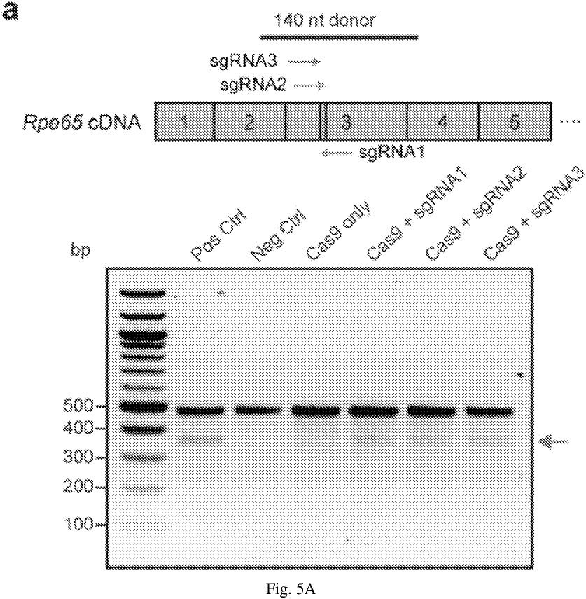

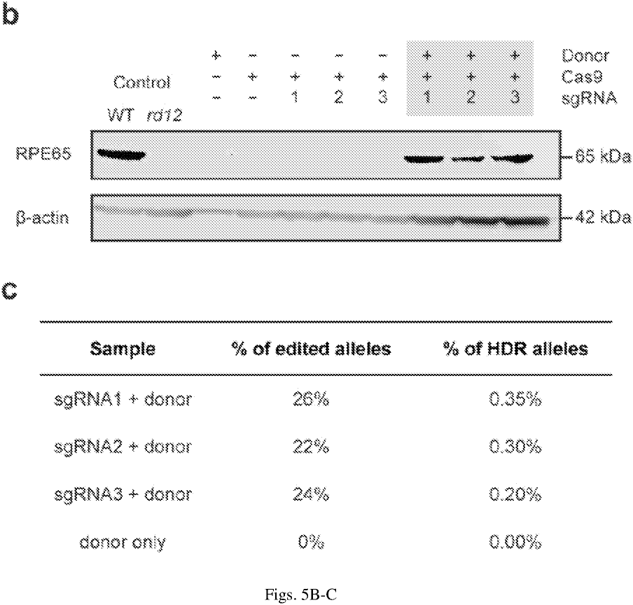

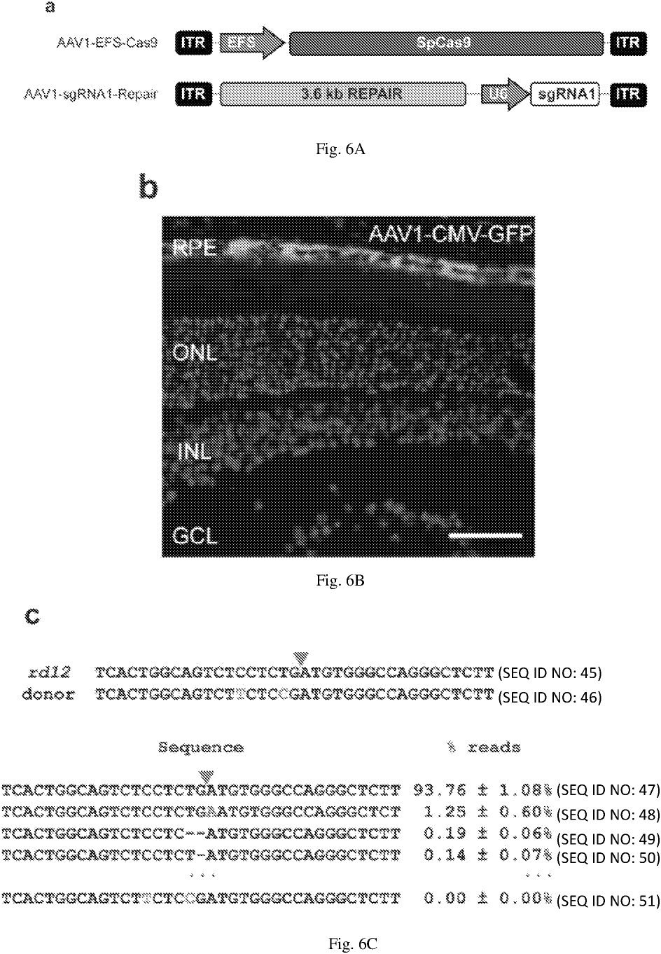

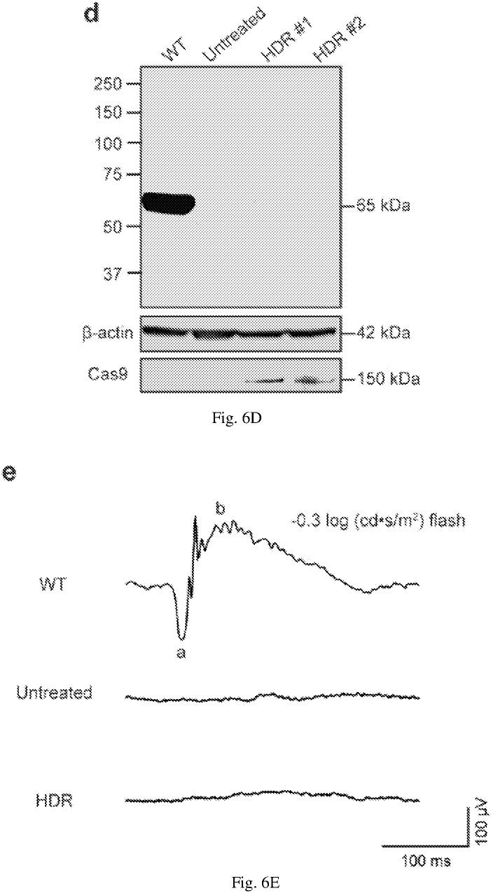

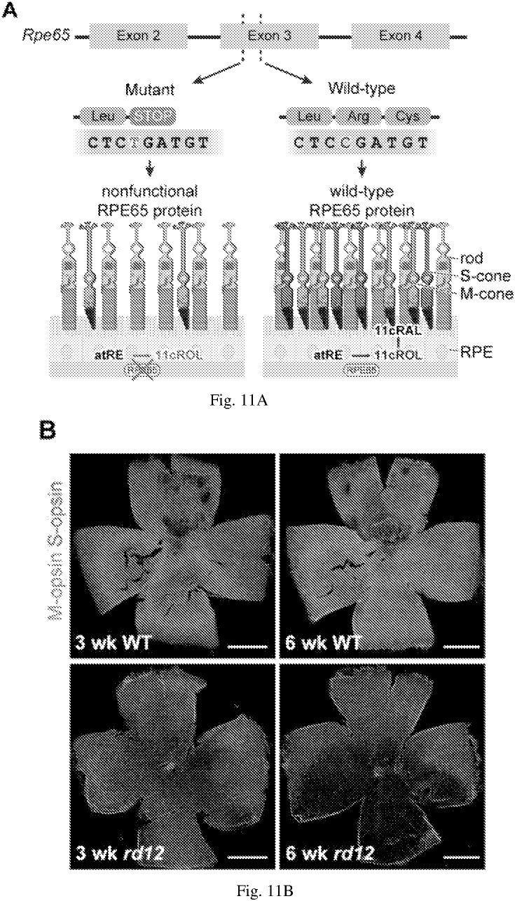

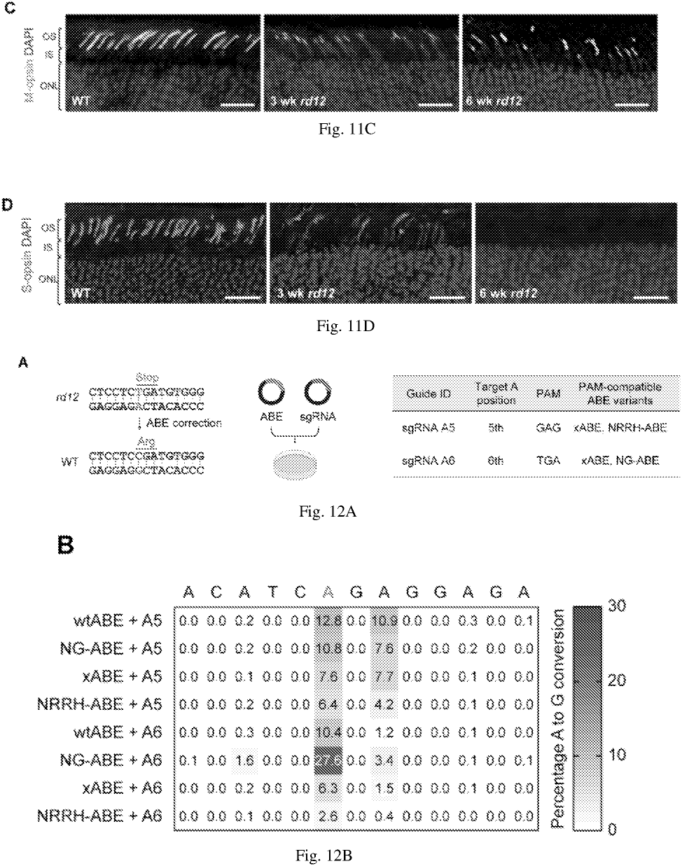

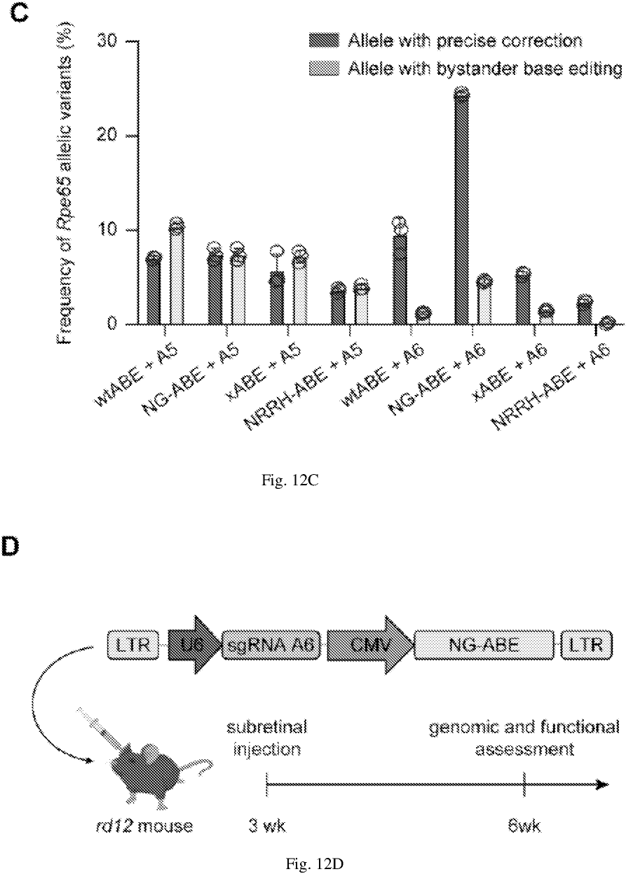

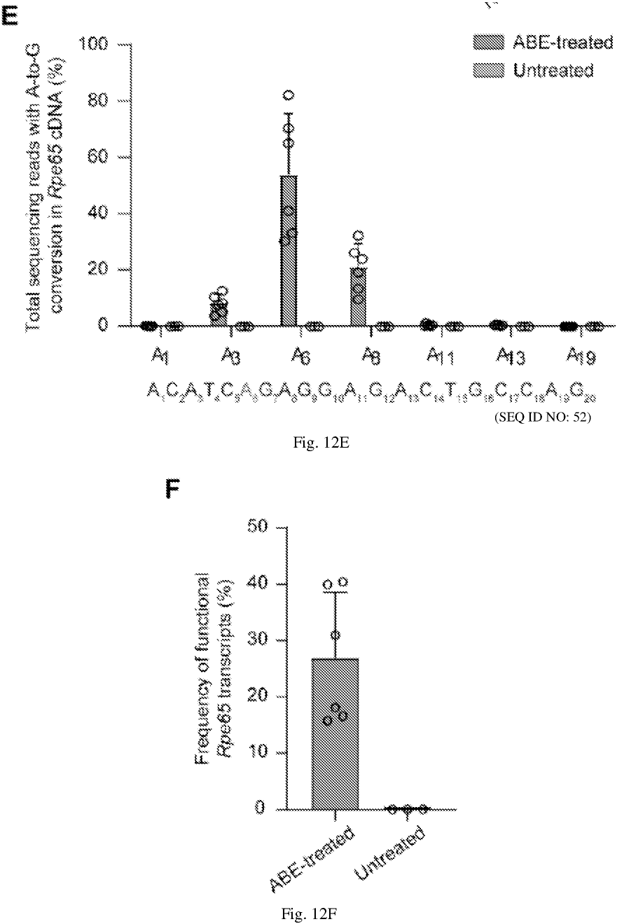

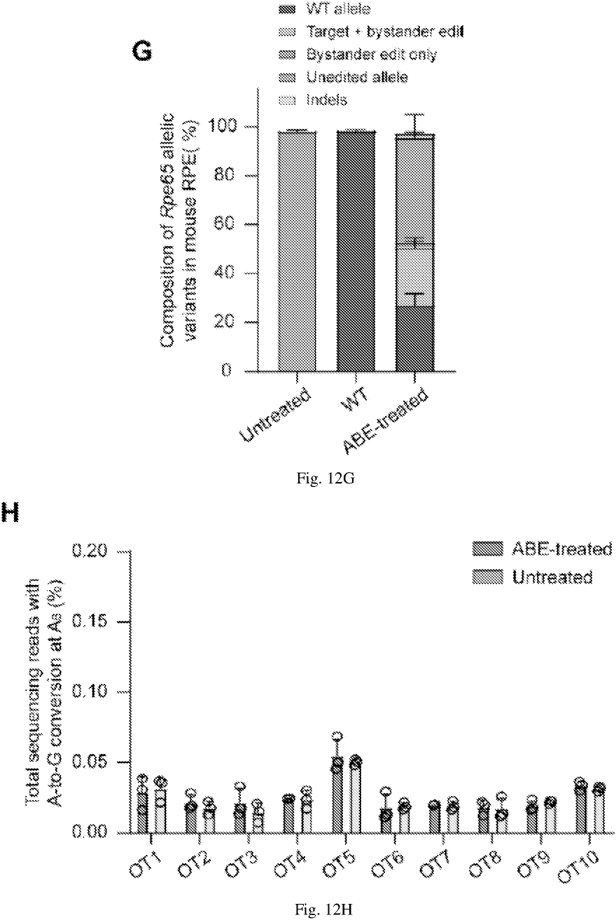

Lastly, we evaluated whether ABE treatment can restore complex cortical visual processing, such as spatial and temporal resolution, and direction and contrast discrimination, in V1 of rd12 mice. We recorded visually evoked responses to flashes of light from single neurons and visually evoked potentials (VEPs) from multiple sites in V1 of WT, untreated rd12, and treated rd12 mice. The typical flash-evoked responses along with average population histograms are shown in FIGS. 4A-D. The representative VEP examples from a single mouse in each group are shown in FIG. 11A. The comparison of normalized population VEP amplitudes in treated rd12 mice showed 79% recovery (1.48 ± 0.19 µV) of that found in WT mice (1.90 ± 0.22 µV) with no statistical difference between the two groups (FIGS. 11B, C). Conversely, in untreated rd12 mice, we found no visually evoked responses from single neurons or VEP amplitudes above noise signal (0.20 ± 0.02 µV) (FIG. 4B and FIGS. 11B, C). We also observed improved average response latencies in treated animals, although the difference between WT and untreated animals was narrow and the statistically significant difference from either group was not achieved by the treated animals (FIG. 11C). In single V1 cell recordings, we found that the ABE-treated rd12 mice not only displayed flash evoked responses, but also showed strong selective responses to various parameters such as direction, spatial and temporal frequency tuning, receptive field size, and contrast (FIGS. 4e-i, respectively). The population averages for each parameter were comparable to the WT controls, although the responses in the V1 cell population of treated rd12 animals were slightly reduced compared to those from WT animals (FIGS. 12A-F).

In summary, we demonstrate the therapeutic application of ABE to correct RPE65 gene mutations and cure blindness. We optimized the base editing outcome using in vitro platform, successfully translated the system to the animal model and performed the comprehensive visual function assessment after treatment. We used LV vector for this initial proof-of-concept study, recognizing that safer alternative delivery methods, such as split intein- AAV vectors, will need to be optimized for translation to the clinic. Nevertheless, our study provides a framework for the preclinical development of a base-editing therapeutic for other genetic diseases. Importantly, we provide the first evidence that ABE can efficiently correct a mutation site lacking canonical NGG PAM at clinically relevant level, suggesting the expanded applicability of ABE to target a larger number of pathogenic mutations.

Gene therapy approaches to treating inherited retinal diseases are of special interest given the accessibility of the eye, its immune-privileged status and the successful clinical trials of RPE65 gene augmentation therapy which led to the first FDA-approved gene therapy. This landmark therapeutic advance was made possible through the work of numerous laboratories in the US and England. Now, as demonstrated in this example, base editing technology could provide an alternative to gene augmentation therapy to permanently rescue the function of a key vision-related protein disabled by mutations, or to correct dominant alleles for which gene augmentation may not be effective. This work represents a critical advance towards development of treatment for many inherited retinal diseases.

Although we only demonstrated correction of the R44X mutation in this study as proof of concept, a large fraction of IRD-associated mutations could theoretically be corrected with base editors. A wide variety of engineered base editor variants have been described (including SpCas9-NRRH, -NRCH, -NRTH, -NG, -NY, and -NR) and are no longer practically constrained by the requirement of PAM for sequence recognition and enable base editing of a previously inaccessible pathogenic single-nucleotide polymorphism (SNP) (FIG. 2G). Of all pathogenic SNPs in the ClinVar database, -95% of transition mutations (equivalent to 62% of all point mutations) are targetable with the set of engineered cytosine or adenine base editors (FIG. 2F). As significant progress is made toward the development of better base editor variants that have broader PAM compatibility, higher editing efficiency and less off-target effects, we believe that base editor approaches have great clinical potential for the treatment of numerous inherited retinal diseases caused by different mutations.

### Example 2

In this Example, we investigated whether base editing treatment can rescue the function and survival of cone photoreceptors in the rd12 mouse, which shows a rapid degeneration of cone photoreceptors. Because protecting photoreceptors is a key to prevent further deterioration of vision in LCA patients, this Example assesses the therapeutic potential of base editing as a one-time, durable treatment for LCA2.

### Materials and Methods

### Mice

The pigmented rd12 mice and C57BL/6J mice were purchased from the Jackson Laboratory (Jackson Laboratory; 005379 and 000664, respectively). Gnat1^{-/-} mice were the generous gift from Janet Lem (Tufts University, Boston). Rd12Gnat1^{-/-} mice were generated by crossbreeding Gnat1^{-/-} mice with rd12 mice. Progeny were genotyped as described previously. The homozygosity of rd12 mutation was validated by Transnetyx genotyping. All mice were housed in the vivarium at the University of California, Irvine, where they were maintained on a normal mouse chow diet and a 12/12-h light/dark cycle. All animal procedures were approved by the Institutional Animal Care and Use Committee (IACUC) of the University of California, Irvine, and were conducted in accordance with the Association for Research in Vision and Ophthalmology Statement for the Use of Animals in Ophthalmic and Visual Research.

### Cell Line Generation

Stable cell line expressing a mouse RPE65^{rd12} variant was generated by transduction of NIH3T3 cells with retrovirus obtained from Phoenix-Eco cells transfected with pMXs-RPE65(rd12)-IRES-GFP according to a previously published protocol.

### In Vitro Base Editing Validation

NIH3T3-RPE65 (rd12) cells were seeded on a 24-well plate 18 h prior to transfection. At ~70% confluency, cells were transfected with 750 ng of ABE-expression plasmid and 250 ng of sgRNA-expression plasmid using 1.5 µl of Lipofectamine 3000 (Thermo Fisher, no. L3000001) per well. Four kinds of ABE-expression plasmids include: pCMV-ABEmax (Addgene plasmid #112095), NG-ABEmax (Addgene plasmid #124163), xABEmax (Addgene plasmid #119813) and pCMV-ABEmax-NRRH. Two sgRNA-expression plasmids were generated as previously described. Cells were harvested for genomic DNA purification 48 h post-transfection.

### Lentivirus Generation for in Vivo ABE Delivery

To generate a single lentiviral vector co-expressing sgRNA-A6 and NG-ABEmax, the lentiviral transfer plasmid, LV-ABEmax-A6, generated from previous study⁸ was double-digested with EcoRI and Eco32I uI to replace 2,284-bp sequence with the homologous sequence from NG-ABEmax (Addgene plasmid #124163), double-digested with EcoRI and Eco32I. The final cloned plasmid was packaged into lentivirus particles by Signagen.

### Adeno-Associated Virus Generation for in Vivo ABE Delivery

N-terminal ABE7.10 AAV is identical to that published in previous study. To replace the C-terminal AAV plasmid Cas9 variant with SpCas9-NG, first SpCas9-NG was amplified from NG-ABEmax (Addgene plasmid #124163) with the following primers: Forward: TGCTTCGACTCCGTGGAAATCTC (SEQ ID NO: 35) and Reverse: GACTTTCCTCTTCTTCTTGGGC (SEQ ID NO: 36), and the resulting product was cloned via Gibson assembly into C-terminal ABE7.10 AAV from previous study that was cut with PasI and EcoRI. After sequence confirmation, the plasmid was digested overnight with BsmBI to insert the guide sequence. The guide sequence was ordered as two oligos which were annealed and phosphorylated in vitro before ligation into the cut vector using T4 DNA ligase. The sequence of the forward oligo encoding the guide sequence was CACCGACATCAGAGGAGACTGCCAG (SEQ ID NO: 37) and AAACCTGGCAGTCTCCTCTGATGTC (SEQ ID NO: 38). Adeno associated virus expressing the split base editor was produced using the previously described protocol. Briefly, HEK293T/17 cells were plated in 15 cm dishes to about 80-85% confluency 24 h before transfection. Cells were then transfected with PEI containing 5.7 µg AAV genome, 11.4 µg pHelper (Clontech), and 22.8 µg of rep-cap plasmid per 15 cm dish. Media was changed to DMEM with 5% FBS one day after transfection. The virus was then extracted from cells 72 hours after transfection from both the cell lysate and the supernatant. All the viruses were purified with an iodixanol step gradient using Ti 70 fixed angle rotor at 58,600 rpm for 2 hours 15 mins at 4° C. Ultracentrifugation was followed with buffer exchange and concentration step using 100-kD MWCO columns (EMD Millipore). The concentrated viral solution was sterile-filtered using a 0.22 µm filter, and stored at 4° C. until use. All viruses were titered via quantitative PCR using the AAVpro Titration Kit v.2 (Clontech), following the manufacturer’s protocol.

### Deep Targeted Sequencing Analysis

Genomic DNA (gDNA) from cultured cells or mouse RPE tissue was isolated using the DNeasy Blood and Tissue Kit (Qiagen, no. 69504) according to the manufacturer’s instructions. Complementary DNA (cDNA) was synthesized from the total RNA extracted from mouse posterior eye cup using Allprep DNA/RNA Mini Kit (Qiagen, no. 80284). Superscript III first-strand synthesis SuperMix (Thermo Fisher Scientific, no. 18080400) was used to synthesize cDNA according to the manufacturer’s instructions. From gDNA and cDNA templates, 200 - 300 bp PCR amplicons of on- and off-target predicted sites for Rpe65 were generated using primers with partial Illumina adapter sequences and then purified using the QIAquick PCR Purification Kit (Qiagen, no. 28106). Samples were sequenced on an Illumina Miseq. Between 70,000 and 100,000 NGS reads for each sample were generated on single-end (1 x 150 bp) or paired-end (2 x 250 bp) run.

### CIRCLE-Seq Off-Target Editing Analysis

Genomic DNA from rd12 mouse tissue was isolated using Gentra Puregene Tissue Kit (Qiagen, no. 158667) according to manufacturer’s protocol. CIRCLE-seq was performed as previously described. Briefly, purified genomic DNA was sheared with a Covaris S2 instrument to an average length of 300 bp. The fragmented DNA was end repaired, A tailed and ligated to an uracil-containing stem-loop adaptor, using KAPA HTP Library Preparation Kit, PCR Free (KAPA Biosystems). Adaptor ligated DNA was treated with Lambda Exonuclease (NEB) and E. coli Exonuclease I (NEB) and then with USER enzyme (NEB) and T4 polynucleotide kinase (NEB). Intramolecular circularization of the DNA was performed with T4 DNA ligase (NEB) and residual linear DNA was degraded by Plasmid-Safe ATP-dependent DNase (Lucigen). In vitro cleavage reactions were performed with 250 ng of Plasmid-Safe-treated circularized DNA, 90 nM of Cas9-NG protein, Cas9 nuclease buffer (NEB) and 90 nM of synthetic chemically modified sgRNA (BioSpring), in a 100 µl volume. Cleaved products were A tailed, ligated with a hairpin adaptor (NEB), treated with USER enzyme (NEB) and amplified by PCR with barcoded universal primers NEBNext Multiplex Oligos for Illumina (NEB), using Kapa HiFi Polymerase (KAPA Biosystems). Libraries were sequenced with 150 bp paired-end reads on an Illumina MiSeq instrument. CIRCLE-seq data analyses were performed using open-source CIRCLE-seq analysis software (https://github.com/tsailabSJ/circleseq) using parameters: read_threshold: 4; window_size: 3; mapq_threshold: 50; start_threshold:3; gap_threshold: 3; mismatch_threshold: 6; search_radius: 30; PAM: NG; merged_analysis: True. The mouse genome GRCm38 was used for alignment.

### Mouse Subretinal Injection

Mice were anesthetized by intraperitoneal injection of a cocktail consisting of 20 mg/ml ketamine and 1.75 mg/ml xylazine in phosphate-buffered saline at a dose of 0.1-0.13 ml per 25 g body weight, and their pupils were dilated with topical administration of 1% tropicamide ophthalmic solution (Akom, no. 17478-102-12). Subretinal injections were performed using an ophthalmic surgical microscope (Zeiss). An incision was made through the cornea adjacent to the limbus at the nasal side using a 26-gauge needle. A 35-gauge blunt-end needle (World Precision Instruments, no. NF35BL-2) connected to an RPE-KIT (World Precision Instruments, no. RPE-KIT) by SilFlex tubing (World Precision Instruments, no. SILFLEX-2) was inserted through the corneal incision while avoiding the lens and pushed through the retina. Each mouse received 1 µl of injection compound per eye. We kept for further evaluation only those injected mice that had more than 95% retinal detachment after subretinal injection and with minimal complications.

### Western Blot Analysis

To prepare the protein lysate from the mouse RPE tissue, the dissected mouse eyecup, consisting of RPE, choroid and sclera, was transferred to a microcentrifuge tube containing 30 µl of RIPA buffer with protease inhibitors, and homogenized with a motor tissue grinder (Fisher Scientific, no. K749540-0000) and centrifuged for 30 min at 20,000 x g at 4° C. The resulting supernatant was pre-cleared with Dynabeads Protein G (Thermo Fisher, no. 10003D) to remove contaminants from blood prior to gel loading. Twenty µl of rd12 cell lysates (15 µl for RPE lysates) were mixed with NuPAGE LDS Sample Buffer (Thermo Fisher, no. NP0007) and NuPAGE Sample Reducing Agent (Thermo Fisher, no. NP0004) and incubated at 70° C. for 10 min, and separated using a NuPAGE 4-12% Bis-Tris gel (Thermo Fisher, no. NP0321BOX) and transferred onto PVDF membrane (Millipore, no. IPVH00010), followed by 1 h blocking in 5% (w/v) non-fat milk in PBS containing 0.1 % (v/v) Tween 20 (PBS-T). The membrane was incubated with primary antibody diluted in 1% (w/v) non-fat milk in PBS-T overnight at 4° C. Primary antibodies include mouse anti-RPE65 monoclonal antibody (1:1,000; in-house production); mouse anti-Cas9 monoclonal antibody (1:1,000; Invitrogen, no. MA523519); rabbit anti-β-actin polyclonal antibody (1:1,000; Cell Signaling Technology, no. 4970S). After overnight incubation, membranes were washed three times with PBS-T for 5 min each and then incubated with secondary antibody for 1 h at room temperature. Secondary antibodies include goat anti-mouse IgG-HRP antibody (1:5,000; Cell Signaling Technology, no. 7076S) and goat anti-rabbit IgG-HRP antibody (1:5,000; Cell Signaling Technology, no. 7074S). After washing the membrane three times with PBS-T for 5 min each, protein bands were visualized after exposure to SuperSignal West Pico Chemiluminescent substrate (Thermo Fisher, no. 34580).

### Immunohistochemistry of Retinal Flatmounts and Cone Quantification

Mouse eyes were fixed with 4% paraformaldehyde in PBS (Santa Cruz Biotechnology, no. 30525-89-4) for 1 h at room temperature and washed three times in PBS for 10 min each. To make retina flatmounts, the retina tissue was separated from the anterior segment and posterior eyecup under dissecting microscope, and four radial cuts were made toward the optic nerve head to flatten the retina. Retinal flatmounts were washed in wash buffer containing 0.5% Triton X-100 (Sigma-Aldrich, no. X100-5 ML) three times for 5 min. To stain cone photoreceptors, retinal flatmounts were incubated with 5% normal donkey serum (Millipore Sigma, no. S30-100 ML), polyclonal goat anti-S-opsin (1:500; custom-made by Bethyl Laboratories) and polyclonal rabbit anti-M-opsin (1:500; Novus Biologicals, no. NB110-74730) antibodies in wash buffer for 3 nights at 4° C. Samples were washed three times for 5 min each and incubated with secondary antibodies, Alexa Fluor 488-conjugated donkey anti-goat IgG (1:250; Abcam, no. ab150129) and Alexa Fluor 647-conjugated donkey anti-rabbit IgG (1:250; Abcam, no. ab150075), for 2 h at room temperature in the dark. After final washing, samples were mounted on slides with VECTASHIELD antifade mounting medium (Novus, no. H-1000-NB). To count the number of S-cones and M-cones in a retinal flatmount, we took 5 images each at dorsal and ventral retina, approximately 1 mm away from the optic nerve using 40X objective lens in Keyence BZ-X810 All-in-One fluorescence microscope. Each quadrant was captured with GFP and Cy5 filters to distinguish S-opsin and M-opsin, respectively. The automated cone quantification in each quadrant was performed using ImageJ software. All images were converted to RGB stack, and the size and intensity threshold was set to identify cone-opsin positive cells.

### Immunohistochemistry of Retinal Cryosections

Following enucleation, the cornea and lens were carefully removed under dissecting microscope while maintaining the shape of the eyecup. The eyecup was fixed with 4% paraformaldehyde in PBS (Santa Cruz Biotechnology, no. 30525-89-4) for 2 h and washed with 5% sucrose in PBS three times for 5 min. The eyecup was dehydrated with 20% sucrose in PBS, embedded in 20% sucrose in O.C.T. (1:2 volume ratio, Sakura, no. 4583), and then flash-frozen for cryosectioning at 10 µm thickness. For immunostaining, cryosections were first blocked with 5% normal donkey serum in 0.2% Triton X-100 in PBS, and then incubated with primary antibodies overnight at 4° C. The S-opsin and M-opsin antibodies were identical to those used in retinal flatmount staining. Cone arrestin was probed with polyclonal rabbit anti-cone arrestin antibody (1:400; Millipore Sigma, no. AB15282). Cone sheaths were stained with fluorescein-conjugated peanut agglutinin (1:200; Vector Laboratories, no. FL-1071). Secondary antibodies were identical to those used in retinal flatmount staining. After incubation for 2 h at RT with secondary antibodies, cryosections were washed three times before placing coverslip with mounting medium with DAPI (Vector Laboratories, no. H-1500-10).

### Electroretinography (ERG)

Scotopic ERG recording was performed as previously described⁸. For photopic ERG recordings, mice were kept in a lighted vivarium. After induction of anesthesia, pupils were dilated with 1% tropicamide (Henry Schein, no. 1127192), and applied with 2.5% hypromellose (Akom, no. 9050-1) to keep corneas hydrated. A mouse was placed on a heated Diagnosys Celeris rodent ERG device (Diagnosys LCC), and the ocular electrodes and ground electrode were placed on the corneas and hind leg, respectively. To measure M-cone and S-cone function, stimulation was performed with alternating green light and UV light at increasing intensities. Green light stimulation (peak emission 544 nm, bandwidth 160 nm) had intensity increments of 0.3, 3, 30 and 300 cd· s/m². UV light stimulation (peak emission __ nm, bandwidth) had intensity increments of 0.1, 1, 10 and 100 cd·s/m². The responses for 20-25 stimuli with an inter-stimulus interval of 2-5 s were averaged together, and the a-and b-wave responses were acquired from the averaged ERG waveform. The ERGs were recorded with the Celeris rodent electrophysiology system (Diagnosys LLC) and analyzed with Espion V6 software (Diagnosys LLC).

### Single-Cell RNA-Seq Analysis

Mice were euthanized, and eyes were enucleated for retina tissue isolation. Retinal cells were dissociated using the Papain Dissociation System (Worthington Biochemical) following the manufacturer’s instructions, and diluted at a final concentration of 1,000 cells/µl. In each experimental group, four retinas were used for the mouse single-cell RNA-seq (scRNA-seq). For each group, freshly dissociated cells (~16,500) were loaded into a 10x Genomics Chromium Single Cell system using v2 chemistry following the manufacturer’s instruction. Libraries were pooled and sequenced on Illumina NovaSeq6000 with -500 million reads per library. Sequencing results were processed through the Cell Ranger 5.0.1 pipeline (10× Genomics) with default parameters. Seurat version 3.1 (90) was used to perform downstream analysis following the standard pipeline using cells with more than 200 genes and 1000 UMI counts, resulting in 4,240 WT mouse cells, 7,482 untreated rd12 cells, and 5,174 treated rd12 cells. Samples were aggregated, and cell clusters were annotated based on previous literature. UMAP dimension reduction was performed on the top principal components learned from high variance genes. Gene expression of each cell cluster was calculated using the average expression function of Seurat. Gene differential expressions of each cell type among different groups were performed using FindMarkers function with Wilcoxon test in Seurat.

### Primary Visual Cortex (V1) Electrophysiology

Mice were initially anesthetized with 2% isoflurane in a mixture of N₂O/O₂ (70%/30%) then placed into a stereotaxic apparatus. A small, custom-made plastic chamber was glued to the exposed skull. One day after recovery, re-anesthetized animals were placed in a custom-made hammock, maintained under isoflurane anesthesia (1-2% in a mixture of N₂O/O₂) and onto four individual tungsten electrodes were inserted into a small craniotomy above the visual cortex of the right hemisphere. Once electrodes were inserted, the chamber was filled with sterile agar. During recording sessions, animals were sedated with chlorprothixene hydrochloride (1 mg/kg; IM) and kept under light isoflurane anesthesia (0.2 - 0.4%). EEG and EKG were monitored throughout the experiments and body temperature was maintained with a heating pad (Harvard Apparatus).

Data was acquired using a 32-channel Scout recording system (Ripple). The local field potential (LFP) from multiple locations was band-pass filtered from 0.1 Hz to 250 Hz and stored together with spiking data on a computer with 1 kHz sampling rate. The LFP signal was cut according to stimulus time stamps and averaged across trials for each recording location to calculate visually evoked potentials (VEP). The spike signal was band-pass filtered from 500 Hz to 7 kHz and stored in a computer hard drive at 30 kHz sampling frequency. Spikes were sorted online in Trellis (Ripple) while performing visual stimulation. Visual stimuli were generated in Matlab (Mathworks) using Psychophysics Toolbox and displayed on a gamma-corrected LCD monitor (55 inches, 60 Hz; 1920 x 1080 pixels; 52 cd/m² mean luminance). Stimulus onset times were corrected for LCD monitor delay using a photodiode and microcontroller⁶¹ (in-house design).

For recordings of visually evoked responses, cells were first tested with 300 repetitions of a 500 ms bright flash stimulus (105 cd/m²). The background activity was calculated as average activity from 500 ms before stimulus onset for each repetition.

### Analysis of V1 Electrophysiology

The response amplitude of LFP was calculated as a difference between the peak of the positive and negative components in the VEP wave (Kordecka et al., 2020). The response latency was defined as the time point where maximum response occurred. The maximum of the response was defined as maximum of either the negative or positive peak. The single unit responses to the flash stimulus were compared as the maximum response to stimulus ON-set. Average differences between animal groups were considered statistically significant at P ≤ 0.05 for two-tailed Mann-Whitney U-tests. Mean values given in the results include error bars for the standard error of the mean (SEM). All offline data analysis and statistics were performed in Matlab (Mathworks, USA).

### Results

### Rd12 Mice Exhibit Early Cone Dysfunction and Rapid Cone Degeneration

We first examined the time course of cone degeneration in the rd12 mice to determine an optimal age for treatment. In mouse, cones are classified by two types of light-detecting proteins, S-opsin (short wavelength-sensitive or blue/UV-sensitive) and M-opsin (medium wavelength-sensitive or green-sensitive), which are expressed in an opposing dorsal-ventral gradient. Previous studies have reported that rd12 mice display early cone dysfunction and degeneration from 2 weeks of age, even before complete development of the retina; and extensive loss of S-opsin-positive cones occurs by 5 weeks of age (FIG. 11A). As our goal is to evaluate the ability of base editing to protect cone degeneration in the context of clinical practice, we chose 3 weeks of age as our treatment time point, at which time the retina is fully developed and the process of degeneration has already begun.

At 3 weeks of age, the densities of S-opsin-positive cones (S-cones) and M-opsin-positive cones (M-cones) were already decreased as shown on retinal flatmounts of the rd12 mice, in comparison to those of age-matched wild-type mice (FIG. 11B). Also, the retinal cross-section for the rd12 mice revealed mislocalization of S-opsins and M-opsins to the inner segments, cone nuclei and axons, in contrast to correct localization to the cone outer segments for the wild-type mice (FIGS. 11C, D). By 6 weeks of age, nearly all S-cones have disappeared on the retinal flatmounts (FIGS. 11B, D), while M-cones still remained on the dorsal retina. The retinal cross-section, however, revealed M-opsin mislocalization and shortening of the cone outer segments, indicating the pathological process in the M-cones (FIG. 11C). Based on these findings, we decided to administer the treatment in mice at 3 weeks of age and evaluate the post-treatment outcome after 6 weeks of age.

### Evolved Adenine Base Editor Enhances the Mutation Correction Rate in Vitro

Although we previously found that the adenine base editor (ABE) and sgRNA can correct the rd12 mutation, we now sought to improve the base-editing efficiency by testing other ABE variants that can recognize a wider array of protospacer-adjacent motif (PAM) sequences. The PAM sequence is a short DNA sequence (2-6 base pairs) that follows the DNA sequence targeted by the Cas9 nuclease in the CRISPR bacterial immune system. Therefore, having a correct PAM sequence at the target site is necessary for successful genome targeting. In Example 1, we showed that two single-guide RNAs, sgRNA-A5 (A5) and sgRNA-A6 (A6), which place the mutant base at the 5^{th} and 6^{th} base position of the protospacer respectively, can correct the rd12 mutation with codon-optimized ABE(7.10) coupled to the wild-type nSpCas9 (hereby referred to as wtABE), despite not having the canonical NGG PAM sequence at the targeted site. In the treated animals A5 showed a higher on-target base-editing efficiency, although A6 demonstrated a higher precision with lower bystander base editing. To enhance the on-target correction rate and reduce the bystander base editing, we evaluated three other evolved ABE variants that were shown to be more compatible with the PAM sequences of A5 and A6: codon-optimized NG-ABE, xABE and NRRH-ABE (FIG. 12A). We transfected different combinations of ABE and sgRNA into the cell line, which stably expresses Rpe65^{rd12} cDNA (rd12 cell line), and then analyzed the base editing outcome by deep DNA sequencing.

The sequencing analysis revealed that regardless of the ABE types co-transfection of A5 consistently showed a higher rate of bystander A-to-G conversion than co-transfection of A6, especially at the adenine located 2 bases downstream of the target mutation (FIG. 12B). Among the transfected groups with A5, the wtABE showed the highest on-target base conversion (10.87 ± 0.32%) contrary to our expectation that NRRH-ABE would be more efficient at recognition of A5 PAM (GAG). Among the transfected groups with A6, the NG-ABE showed the highest on-target correction rate (27.64 ± 0.20%), which was the highest of all groups.

Since the true depth of rescue is determined by the relative amount of functional Rpe65 alleles, we examined the percentage of precisely corrected Rpe65 alleles in each transfection group (FIG. 12C). The A6 + NG-ABE group contained 24.36 ± 0.26% of functional Rpe65 alleles, which was the highest percentage among all groups. The A6 + NG-ABE group contained 4.61 ± 0.17% of Rpe65 alleles, which contained bystander base edits. Given its superior correction rate and relatively low bystander editing, we selected the combination of A6 and NG-ABE to test in our animal models.

### Subretinal Delivery of NG-ABE and sgRNA-A6 Improves the Correction Rate

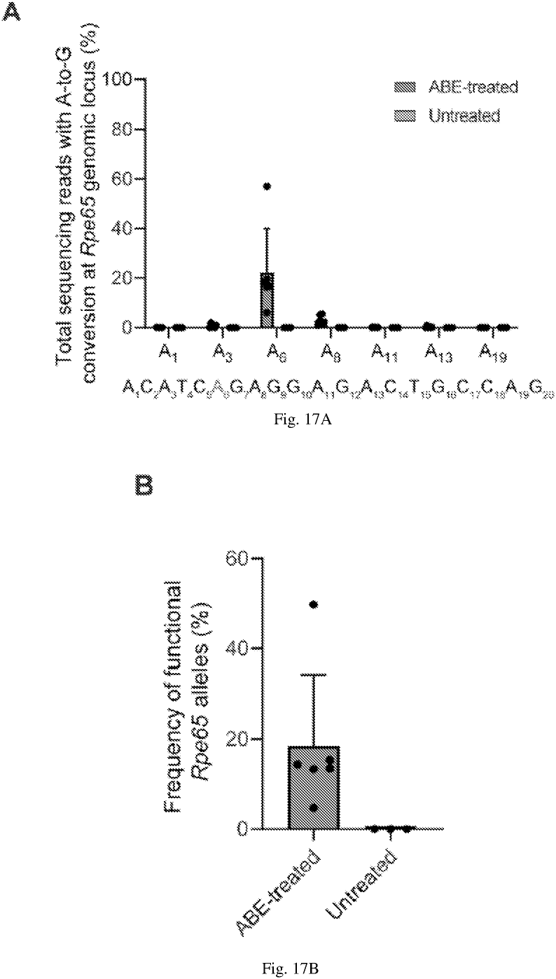

To deliver the NG-ABE and sgRNA-A6 to the mouse RPE, we packaged these expression sequences into a single lentivirus vector (LV-ngABE-A6) (FIG. 12D) and injected the lentivirus subretinally into 3-week-old rd12 mice. At 3 weeks post-injection, we evaluated the outcome of base editing. The genomic DNA analysis from the treated RPE cells showed up to 57% of A-to-G conversion at the target adenine (A₆), with the average correction of 22 ± 18% (n = 6 eyes) (FIG. 17). However, we noted that the process of isolating RPE cells from the posterior eye cup results in variable distribution of RPE cells within each sample due to contamination of other cell types, hindering the DNA analysis within only RPE cells. Therefore, we detached all cells in the posterior eyecup and examined the sequence of Rpe65 cDNA, as Rpe65 is exclusively expressed in the RPE cells.

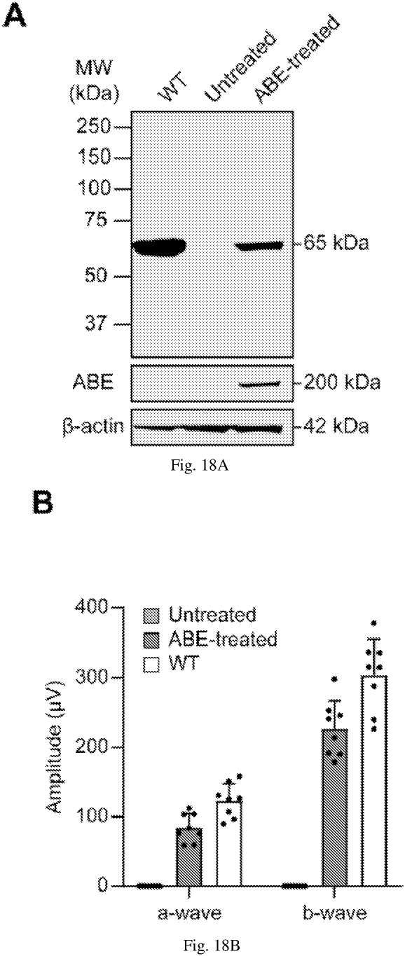

The sequencing analysis of Rpe65 cDNA from the treated eyes revealed up to 82% of A-to-G conversion at the target adenine (A₆) with the average frequency of 54 ± 22% (n = 6) (FIG. 12E). The most frequent bystander editing occurred at As (21 ± 8%), followed by A₃ (8 ± 3%), consistent with the pattern predicted from the in vitro study (FIG. 12B). When we examined the percentage of precisely corrected Rpe65 transcripts within each treated eye, there was up to 40% of functionally rescued alleles in the eye, with the average frequency of 27 ± 12% in all eyes (FIG. 12F). Other modified transcripts were comprised of those containing A-to-G conversion of both mutation and bystander bases (24 ± 9%), or bystander bases only (2 ± 1%) (FIG. 12G). We also sequenced the top ten potential off-target sites identified by the CIRCLE-seq, but did not detect off-target editing above the background level of the untreated eyes (FIG. 12H). We further confirmed the expression of functional RPE65 protein via western blot and the recovery of rod-mediated phototransduction by scotopic electroretinography after dark adaptation (FIG. 18). The treated mice recovered a-wave and b-wave amplitudes of 68% and 74% of the WT responses. AAV-mediated delivery of NG-ABE with sgRNA A6 rescues the phenotype at slower rate

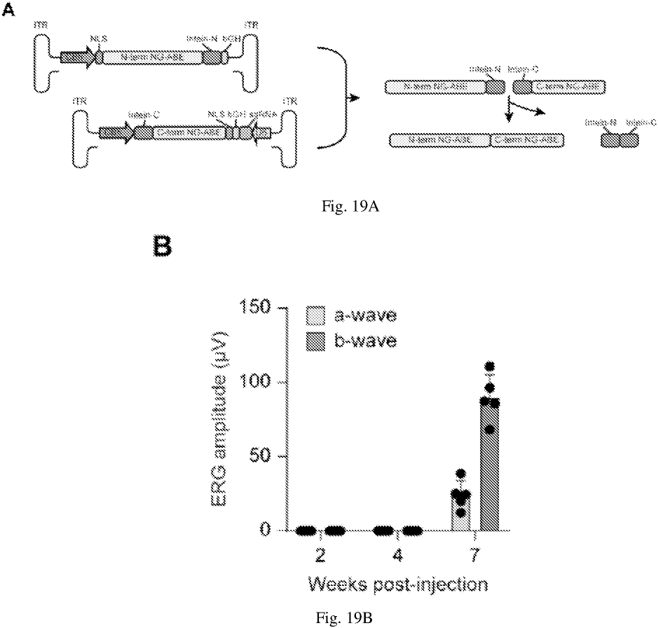

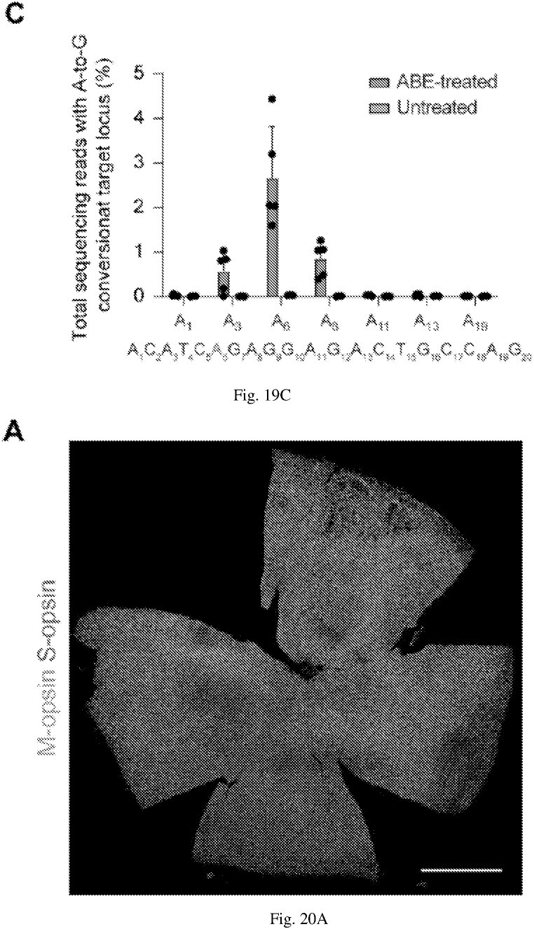

Since adeno-associated virus (AAV) is an ideal vector of choice for gene therapy given its low immunogenicity and favorable safety profile, we also tested targeting the rd12 mutation by packaging NG-ABE and sgRNA-A6 into AAV. Given the limited packaging capacity of AAV, we took advantage of a split base-editor dual-AAV strategy, in which ABE is divided into amino-terminal and carboxy-terminal halves and packaged as two separate AAV serotype 2 vectors (FIG. 19A). When both AAVs transduce the cell, protein splicing in trans would reconstitute a full-length base editor along with the transcription of the sgRNA A6. We first examined the time course of AAV-mediated rescue by measuring the scotopic ERG. The AAV-injected mice did not show detectable ERG responses until 7 weeks after injection, whereas the lentivirus-injected mice showed robust responses one week after injection (FIG. 19B). Genomic DNA analysis of AAV-treated RPE showed relatively low base editing efficiency at the target mutation (2.7 ± 1.2%), but the pattern of base editing was similar to that of lentivirus-treated RPE cells (FIG. 19C). Overall, our findings demonstrate that a split base-editor dual-AAV strategy can also correct the rd12 mutation in mouse RPE cells, although it has a slower mode of action in comparison to the lentivirus. In clinical practice, dual AAV could be a safe approach for base editor delivery. On the other hand, we determined that dual AAV is not feasible for testing our hypothesis due to the rapid degeneration of S-cones in the mouse model. Therefore, we opted for the lentiviral approach to test the ability of base editing for rescuing cone function and survival in mice.

### Base Editing Restores Cone-Mediated Visual Function in the Adult Rd12/Gnat1^{-/-} Mice

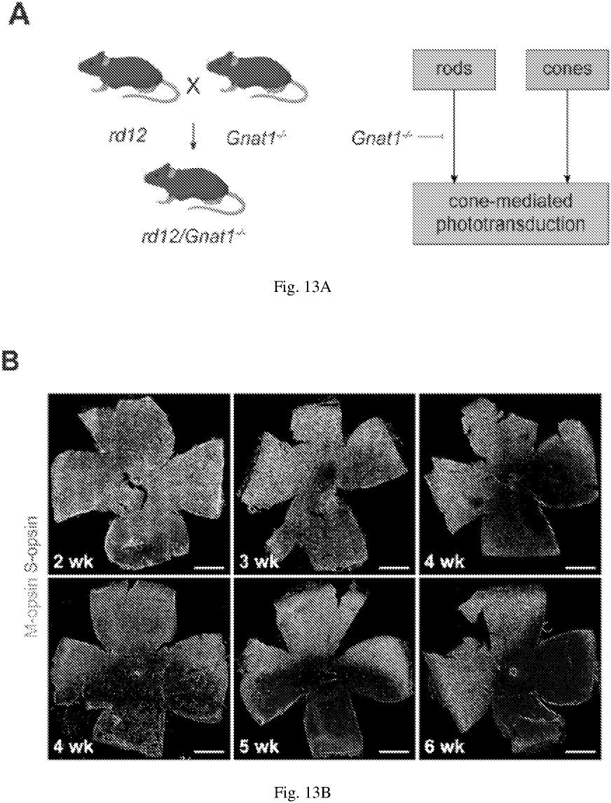

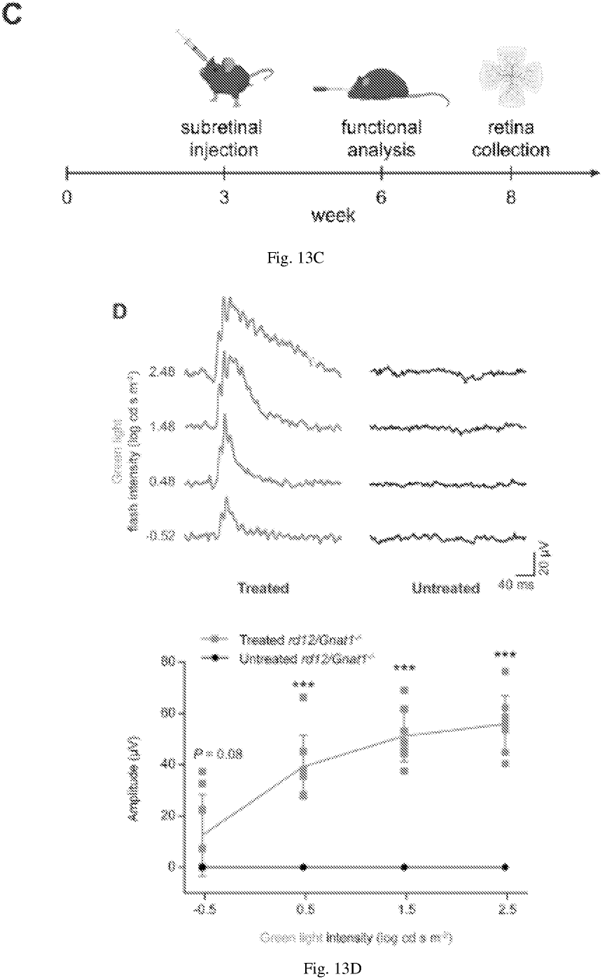

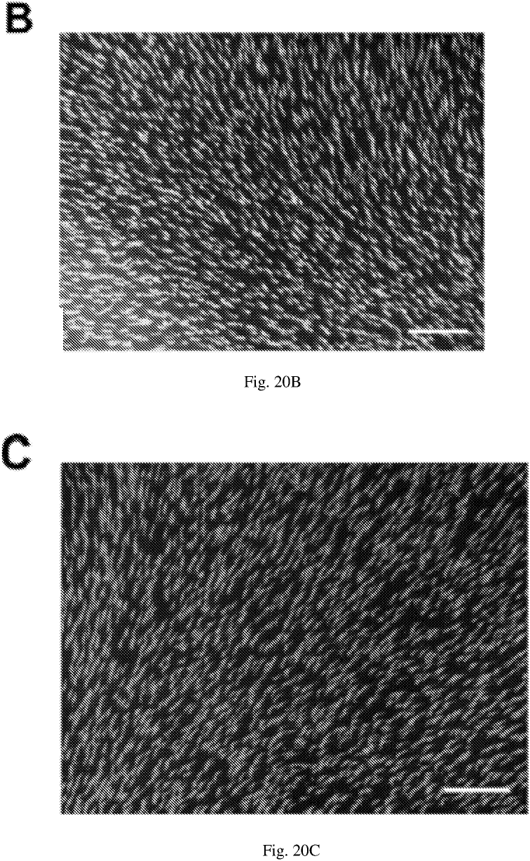

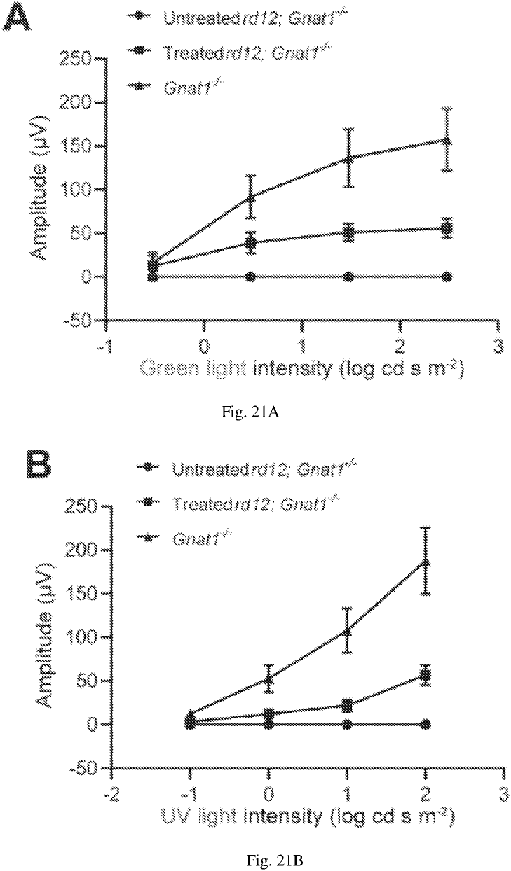

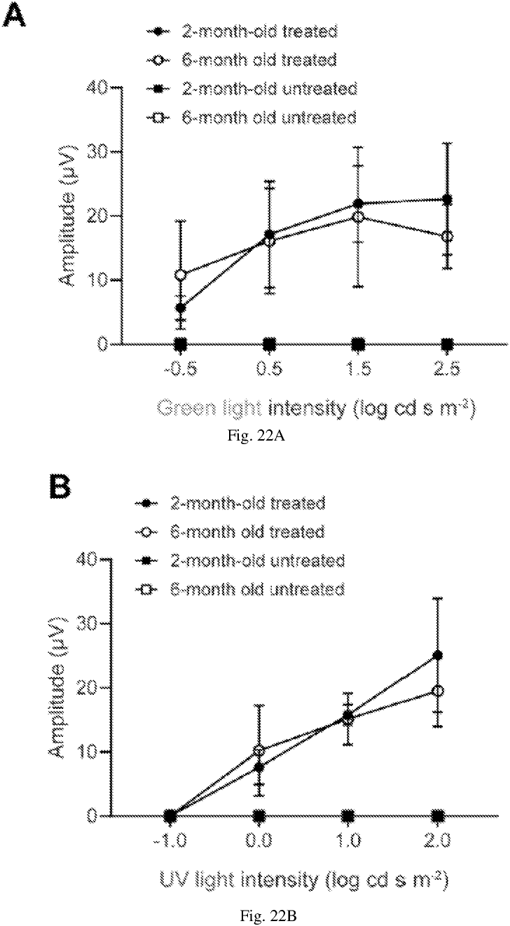

The photoreceptors in the mouse retina are comprised of 98% rods and 2% cones. Because a substantial contribution from rods makes it difficult to measure the visual function mediated by only cones, we abolished the rod-mediated photoresponse by crossing the rd12 mice onto Gnat1^{-/-} mice, which lack rod transducin α-subunit essential for the downstream signal transduction (FIG. 13A). In agreement with previous findings, the knockout of Gnat1 did not have impact on cone structure and survival as shown on the retinal flatmount of the Gnat1^{-/-} mouse (FIG. 20). The rd12/Gnat1^{-/-} mouse showed a similar progression of cone degeneration to the rd12 mouse, with S-cones decreasing from 2 weeks of age, and a complete degeneration by 6 weeks of age (FIG. 13B). We performed subretinal injection of LV-ngABE-A6 into the rd12/Gnat1^{-/-} mice at 3 weeks of age and assessed the cone function with photopic ERG using two distinct wavelengths of light at 6 weeks of age (FIG. 13C). The responses from M-cones were recorded by using a green light stimulus. Photopic ERG waveforms from eyes treated with LV-ngABE-A6 exhibited a prominent b-wave, which increased in amplitude with increasing stimulus intensities, whereas untreated eyes did not respond to any light intensities (FIG. 13D). Similarly, when we recorded the response from S-cones using a UV light stimulus, only the treated eyes showed the ERG amplitudes, which increased with higher intensities (FIG. 13E). The base editing treatment restored approximately 36% of M-cone function (56.0 ± 11.0 µV) and 30% of S-cone function (56.8 ± 11.6 µV), when compared with the age-matched Gnat1^{-/-} eyes (M-cone, 157.5 ± 35.7 µV; S-cone, 187.5 ± 38.1 µV) (FIG. 21).

Furthermore, we measured the functional integrity of the visual pathway from cone via the optic nerves to the visual cortex of the brain by recording visually-evoked potentials (VEPs). The flash stimuli elicited distinct VEP waveforms consisting of three components (an initial negative deflection (N1), a positive deflection (P1) and a more variable negative deflection (N2)) in the control Gnat1^{-/-} and treated rd12/Gnat1^{-/-}mice, but not in the untreated rd12/Gnat1^{-/-} mice (FIGS. 14A, B). However, the VEPs in the treated mice showed attenuated amplitudes and delayed peak times, as compared to that of the control mice (FIGS. 14C, D). The activities of single neurons in the primary visual cortex were also restored following the treatment (FIGS. 14E, F).

### Base Editing Improves the Cone Survival in the Adult Rd12/Gnat1^{-/-} Mice

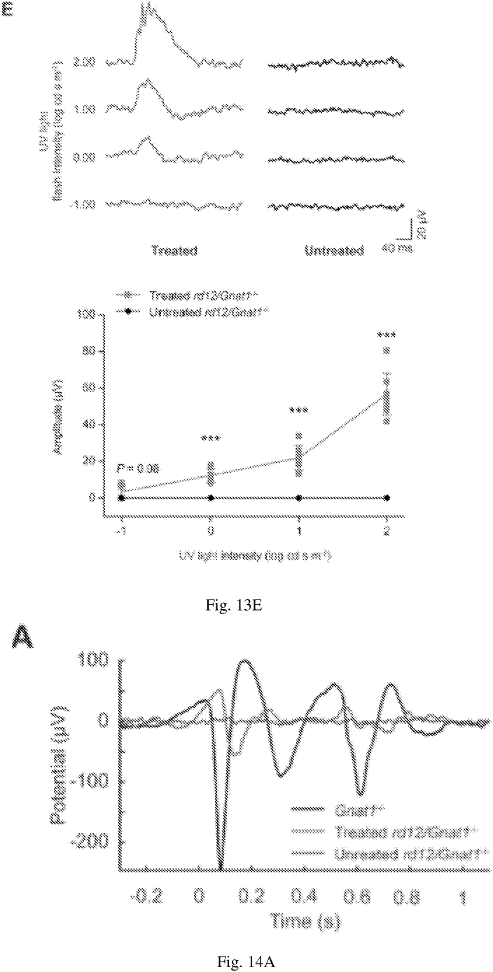

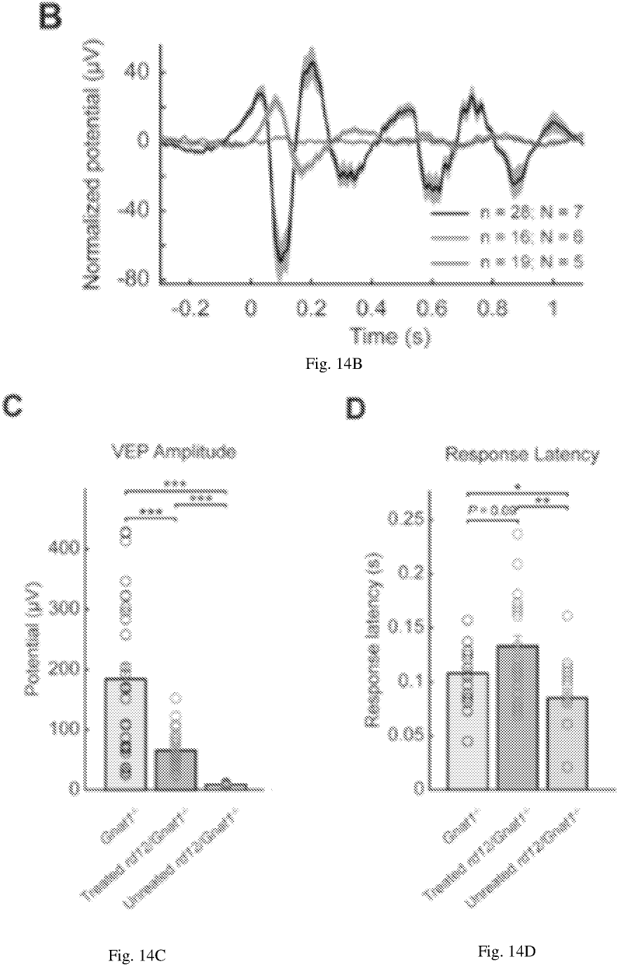

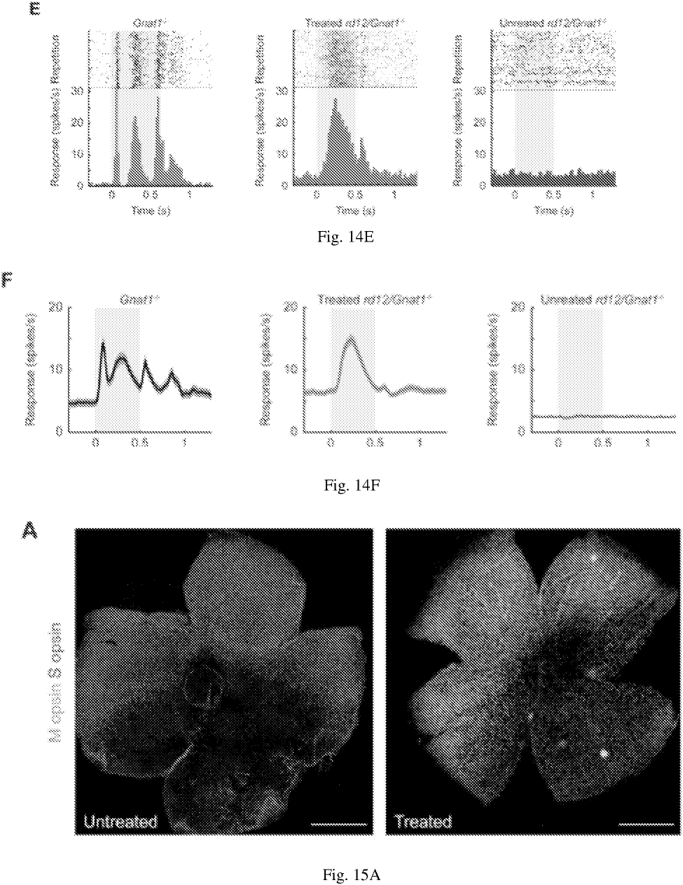

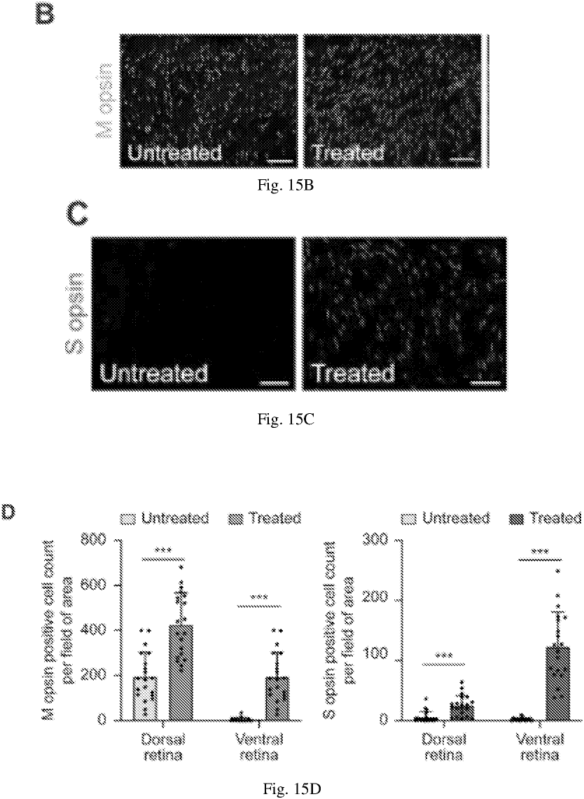

To examine whether base editing prolongs cone survival in rd12/Gnat1^{-/-} mice, we measured the number of M-cones and S-cones on the retina flatmounts at 8 weeks of age, after staining with M-opsin- and S-opsin-specific antibodies. The overall view of retinal flatmounts showed a remarkable preservation of S-cones in the treated eyes in comparison to the untreated eyes (FIG. 15A). We could not discern a difference in M-cones between the treated and untreated eyes from the overall view, but a higher-magnification view of the mid-dorsal retina revealed a decreased density and structural abnormality of M-cones in the untreated retina (FIGS. 15A, B). A higher-magnification view of the mid-ventral retina showed the S-cones in the treated retina, whereas no S-cones were identified in the untreated retina (FIG. 15C). The survival of M-cones and S-cones was quantified by averaging the number of cones in five quadrants across the dorsal and ventral retina at 1 mm from the optic nerve from the treated and untreated eyes (n = 4 per group). In the treated eyes, the average number of S-cones per quadrant was significantly higher in both dorsal (25 vs 5; P < 0.001) and ventral retina (123 vs 2; P < 0.001) compared to the untreated retina (FIG. 15D). The average number of M-cones per quadrant was also significantly higher in the dorsal (426 vs 194; P < 0.001) and ventral retina (24 vs 4; P < 0.001). We also observed that both M-opsins and S-opsins were correctly localized to the cone outer segments in the treated rd12/Gnat1 ^{-/-} mice on retinal cryosections (FIG. 15E).

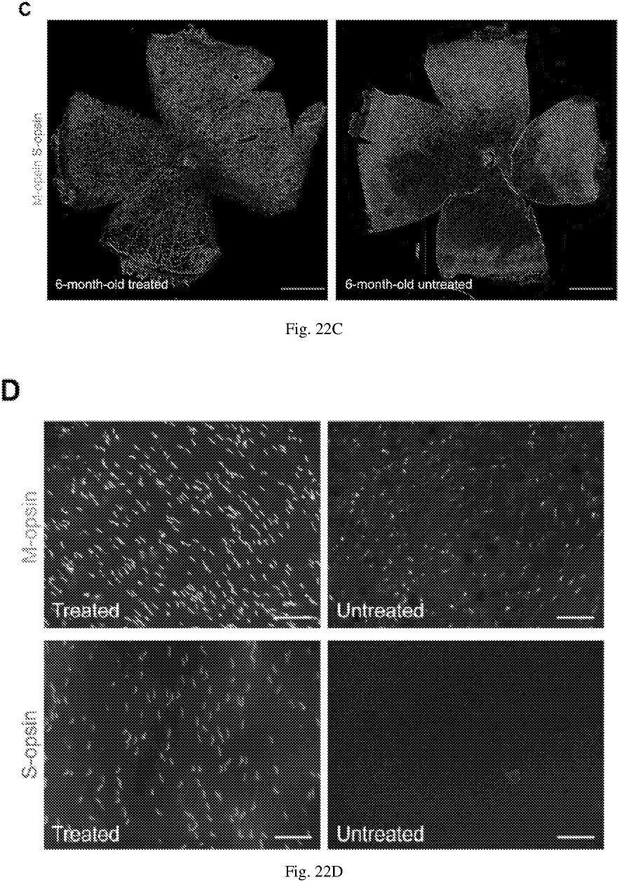

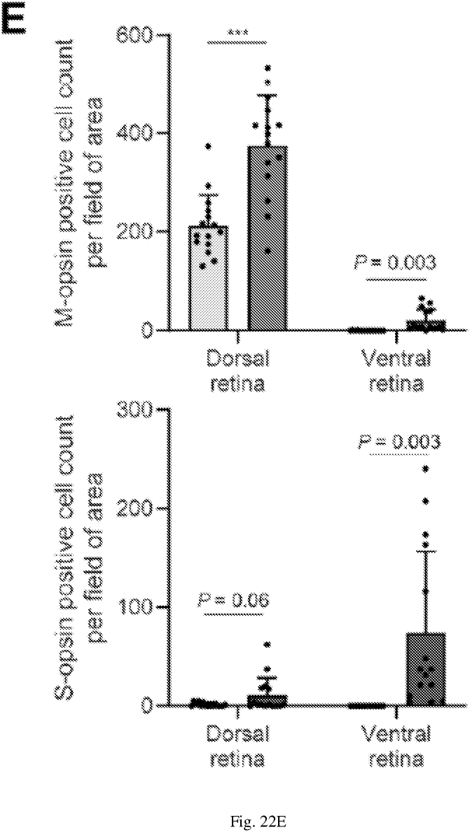

Long-term protection of cone function and structure was also examined with older mice at 6 months of age. ERG recordings from 6-month-old treated mice still displayed the photopic b-waves, indicating M-cone and S-cone function (n = 4 per group) (FIGS. 22A, B). Furthermore, there was no significant decline in the ERG amplitudes between 1.5 and 6 months of age (FIGS. 22A, B). On the retinal flatmounts from the treated eyes, S-cones as well as M-cones were still detected at 6 months of age (FIGS. 22C, D). Cone quantification showed a significantly higher number of M-cones and S-cones in the treated eye, suggesting that base editing is able to prolong cone survival in the long-term (n = 3 per group).

### Base Editing Restores Expression of Cone-Specific Phototransduction Genes

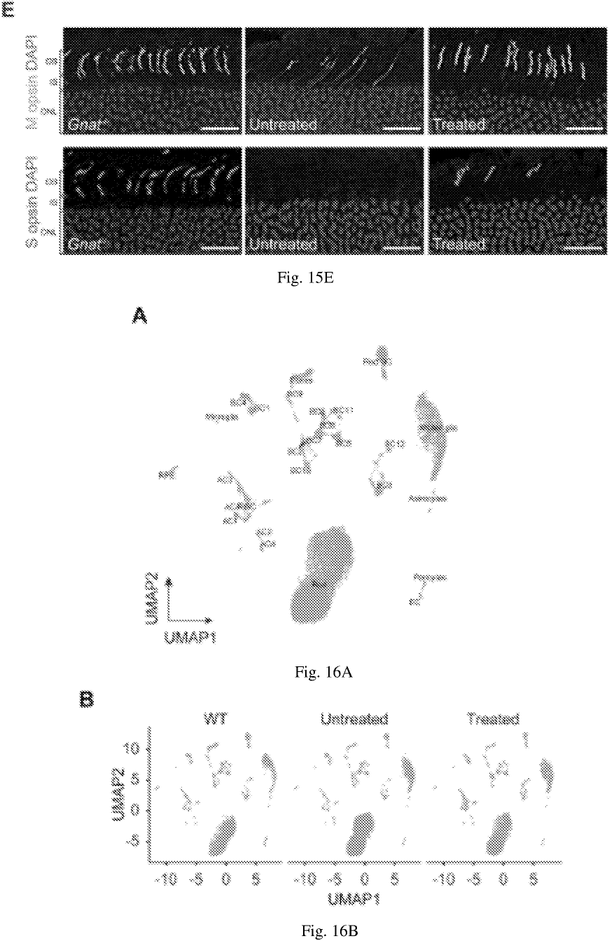

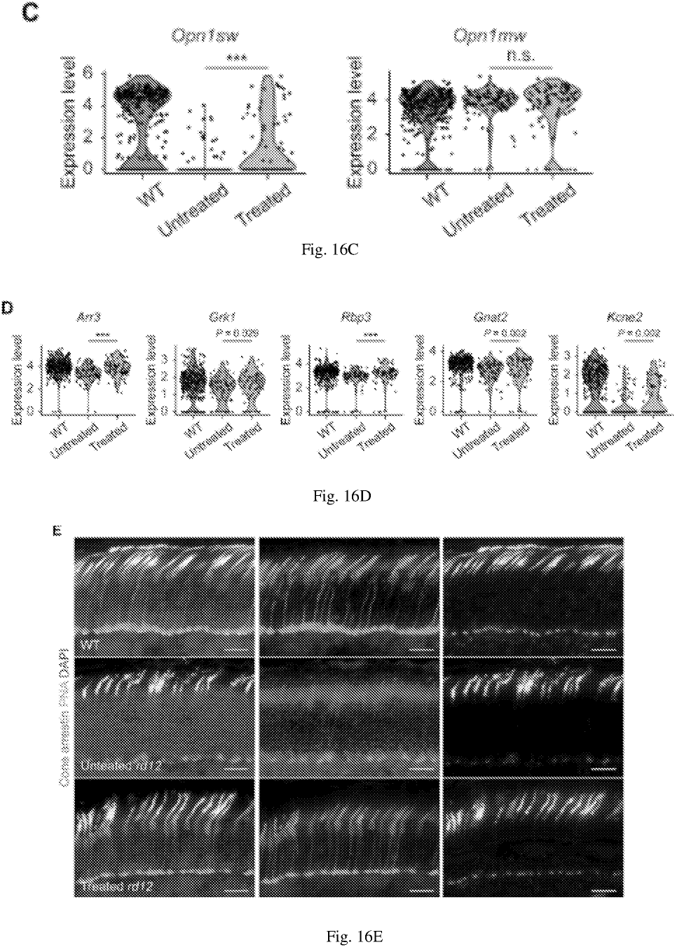

To examine the impact of base editing on the transcriptional rescue of cone photoreceptors, we performed single-cell RNA-sequencing (scRNA-seq) of the retina with 2-month-old control wild-type, and untreated and treated rd12 mice (n = 4 retinas per group). We profiled 16,896 cells from three groups and separated the cells into different clusters which were annotated by expression of cell type-specific marker genes (FIG. 16A). Cell-type distribution was similar across the three groups (FIG. 16B). Cones formed well-defined trajectory, which were identified by expression of the cone-specific marker (Arr3). As predicted, scRNA-seq showed a significantly increased expression level of Opn1sw (S-opsin) in the cone cells of the treated mice in contrast to the cone cells of the untreated mice (P < 0.001) (FIG. 16C, Table 1). Interestingly, the expression level of Opnlmw (M-opsin) was higher in the retinas of both untreated and treated rd12 mice compared to those of wild-type mice. We assume that this result is likely due to the majority of captured cells being M-cones from the rd12 mice as a result of early S-cone cell death (FIG. 16C, Table 1).

We found that expression levels of key genes involved in cone-specific visual phototransduction and bipolar cell synapses (Arr3, Gnat2, Rbp3, Grk1 and Kcne2) were notably downregulated in the cone cells from untreated rd12 mice (FIG. 16D, Table 1). However, these genes were rescued in the treated cone cells (FIG. 16D, Table 1). In particular, cone arrestin, expressed by Arr3, is not only crucial for the regulation of the visual transduction cascade, but also essential for cone survival. Loss of cone arrestin in a knockout mouse model was shown to increase the susceptibility of cones to cell death. Therefore, we evaluated the expression of cone arrestin on the retinal cryosection from treated and untreated rd12 mice at 2 months of age. In untreated rd12 mice, we could not detect cone arrestin even in the live cone cells, labeled with peanut agglutinin (PNA) (FIG. 16E). The treated rd12 mice, on the other hand, showed normal expression of cone arrestin as in the age-matched wild-type mice (FIG. 16E). Therefore, upregulation of Arr3 following the base editing treatment may have implications for long-term protection of cone cells against further degeneration. However, it is yet uncertain whether the altered gene expression is a contributing factor or a concurrent manifestation of the cone degeneration. Nevertheless, the findings from scRNA-seq demonstrated that base editing can reverse the altered gene expression of dysfunctional cones, suggesting its long-term therapeutic benefit for photoreceptor protection.

TABLE 1

| Gene | Protein | Treated | Untreated | WT | p-Value (treated vs. untreated) |
|---|---|---|---|---|---|
| Opn1sw | S-opsin | 27.4 | 2.4 | 61.3 | 1.8E-04 |
| Opn1mw | M-opsin | 61.2 | 55.6 | 43.8 | n.s. |
| Arr3 | Cone arrestin | 58.4 | 34.6 | 60.4 | 3.16E-06 |
| Rbp3 | IRBP | 28.2 | 20.0 | 28.1 | 6.43E-05 |
| Gnat2 | Gnat G(t) submit alpha-2 (cone specific) | 21.5 | 16.2 | 24.4 | 0.00199 |
| Kcne2 | Potassium voltage-gates channel subfamily E member 2 | 2.4 | 1.0 | 5.8 | 0.00172 |
| Grk1 | Rhodopsin kinase | 4.9 | 3.6 | 6.2 | 0.2935 |

Over the past several years, base editing has rapidly emerged as a potential approach to treat genetic disorders with promising outcomes in different preclinical models. Base editing approach especially has great promises for targeting genetic eye disorders, given the unique advantages of the eye (immune privilege, accessibility, anatomical structure) and the prior demonstration of successful genetic rescue in a mouse model. In Example 1 we showed that subretinal delivery of ABE can correct the LCA mutation in a mouse model, suggesting its potential as a treatment. Here, we sought to answer whether base editing can rescue the function and survival of cone photoreceptors from rapid degeneration using a LCA mouse model. Prevention of further retinal degeneration in LCA patients has been a longstanding challenge, and therefore addressing this concern is highly important in development of new therapeutic strategy.

To test our hypothesis, we selected a rd12 mouse model, which displays an early and rapid degeneration of cone photoreceptors and mislocalization of cone opsins due to RPE65 deficiency. Because cone death occurs dramatically in rd12 mouse, it served as a great model to evaluate the effectiveness of base editing in cone protection. In vitro transfection allowed us to predict the outcome of in vivo base editing mediated by different ABE and sgRNA pairs, and to identify the most efficient pair. This finding reveals the importance of screening multiple ABE variants and sgRNAs for each target sequence as a single base difference in sgRNA can have profound impact on the extent of rescue. To evaluate the treatment effects on cone function and survival of mice, we performed subretinal injections into rd12/Gnat1^{-/-} mice, which lack rod-mediated photoresponse, allowing us to measure cone-mediated function alone. Following treatment, we observed a substantial rescue of M-cone and S-cone function by photopic ERG. Furthermore, both M-cones and S-cones were remarkably preserved in treated mice up to 6 months of age, and single-cell RNA-seq of treated retina revealed the restoration of gene expression associated with cone phototransduction and cone survival. These results support that base editing strategy is able to restore cone function, prolong cone survival and transform the gene expression signature of early-onset retinal degeneration.

Several factors may be attributed to the robust rescue of cone photoreceptors by base editing. First, base editing introduces a permanent genomic edit, thereby eliminating the possibility for diminishing expression from an episomal transgene over time. Secondly, it allows more physiologically regulated gene expression, as the corrected gene will be controlled by the endogenous promoter. Lastly, it stops the expression of a truncated, dysfunctional protein, alleviating the potential stress on cells. Taken together, we believe that these factors likely contributed to sustained rescue of cone photoreceptors in the LCA2 mouse model.

In conclusion, we have shown a significant protection against cone loss in a mouse model by base editing. However, additional preclinical testing in larger animals, which have the fovea, would be necessary before this approach can be tested in patients. Furthermore, alternative delivery methods should be investigated to cover a broad area of the RPE tissue in patients and to circumvent constitutive base editor expression, which could induce unwanted DNA editing and immune reaction in the long term. Nevertheless, our results support that base editing could be an effective therapeutic intervention that can rescue and sustain cone photoreceptors in inherited retinal degeneration. We believe base editing will provide new hope for the ultimate cure of inherited blindness.

From the above description of the invention, those skilled in the art will perceive improvements, changes and modifications. Such improvements, changes and modifications within the skill of the art are intended to be covered by the appended claims. All references, publications, and patents cited in the present application are herein incorporated by reference in their entirety.

Having described the invention we claim:

1. A method of treating an inherited retinal disease (IRD) associated with a pathogenic point mutation in a mutant allele of an IRD-related gene in the retina or the retinal pigment epithelium (RPE) of a subject in need thereof, the method comprising:
base editing the pathogenic point mutation in the retinal cell or retinal pigment epithelium cell to correct the pathogenic mutation, generate a non-pathogenic point mutation, or modulate expression of an IRD-related gene and restore visual function of subject.

2. The method of claim 1, where the pathogenic mutation is a nonsense or missense mutation and the base editing increases expression of the protein the retinal cell or retinal pigment epithelium cell by at least about 4%, 5%, 6%, 7%, 8%, 9%, 10%, 20%, 30%, 40% or more.

3. The method of claim 1, wherein the pathogenic mutation is nonsense or missense mutation of an ABCA4, AIPL1, CABP4, CEP290, CLUAP1, CRB1, CRX, GDF6, GUCY2D, IFT140, IQCB1, KCNJ13, LCAS, LRAT, NMNAT1, PRPH2, RD3, RDH12, RHO, RPE65, RPGRIP1, SPATA7, and TULP1.

4. The method of claim 1, wherein the IRD includes at least one of chorioretinal atrophy or degeneration, cone or cone-rod dystrophy, congenital stationary night blindness, Leber congenital amaurosis, macular degeneration, ocular-retinal developmental disease, optic atrophy, retinitis pigmentosa, syndromic/systemic diseases with retinopathy, sorsby macular dystrophy, age-related macular degeneration, doyne honeycomb macular disease, juvenile macular degeneration, Stargardt disease, or retinitis pigmentosis.

5. The method of claim 1, wherein the IRD is Leber congenital amaurosis, Stargardt disease, or retinitis pigmentosis.

6. The method of claim 1, wherein the base editing comprises subretinal injecting at least one vector encoding a base editor and guideRNA that hybridizes to or is complementary to a target nucleic acid sequence, which includes the point mutation, in the IRD-related gene.

7. The method of claim 1, wherein the base editing cause less than 3%, less than 2%, or less than 1% indel formation.

8. The method of claim 6, wherein the pathogenic mutation is nonsense or missense mutation of an RPE65 gene.

9. The method of claim 8, wherein the guide RNA that hybridizes to or is complementary to a target nucleic acid sequence of the mutant RPE65, which includes the point mutation.

10. The method of claim 9, wherein the pathogenic mutation comprises a C to T missense or nonsense mutation of the RPE65 gene and base editing by deamination of the A complementary to the T by the base editor and the guide RNA corrects the C to T mutation.

11. The method of claim 10, wherein the nucleic acid sequence of the target sequence includes at least one of:
5′-CTCACTGGCAGTCTCCTCTGATGTGGGCCA -3′
(SEQ ID NO: 1);
5′-CTCACCGGCAGTCTCCTCTGATGTGGGCCA -3′
(SEQ ID NO:2);
5′- TCACTGGCAGTCTCCTCTGATGTGGGCCAG-3′
(SEQ ID NO: 3);
5′- CACTGGCAGTCTCCTCTGATGTGGGCCAGG-3′
(SEQ ID NO: 4);
5′-ACTGGCAGTCTCCTCTGATGTGGGCCAGGG-3′
(SEQ ID NO: 5);
5′- GCTCACTGGCAGTCTCCTCTGATGTGGGCC-3′
(SEQ ID NO: 6);
5′- GGCTCACTGGCAGTCTCCTCTGATGTGGGC-3′
(SEQ ID NO: 7);
5′- TGGCTCACTGGCAGTCTCCTCTGATGTGGG-3′
(SEQ ID NO: 8);
5′- TCACCGGCAGTCTCCTTTGATGTGGGCCAG-3′
(SEQ ID NO: 9);
5′- CACCGGCAGTCTCCTTTGATGTGGGCCAGG-3′
(SEQ ID NO: 10);
5′-ACCGGCAGTCTCCTTTGATGTGGGCCAGGG-3′
(SEQ ID NO: 11);
5′- GCTCACCGGCAGTCTCCTTTGATGTGGGCC-3′
(SEQ ID NO: 12);
5′- GGCTCACCGGCAGTCTCCTTTGATGTGGGC-3′
(SEQ ID NO: 13); or
5′- TGGCTCACCGGCAGTCTCCTTTGATGTGGGC-3′
(SEQ ID NO: 14).

12. The method of claim 10, wherein the nucleic acid sequence of DNA encoding the guide sequence includes at least one of:
5′-ATCAGAGGAGACTGCCAGTG-3′
(SEQ ID NO: 15),
5′-CATCAGAGGAGACTGCCAGT-3′
(SEQ ID NO: 16),
5′-ACATCAGAGGAGACTGCCAG-3′
(SEQ ID NO: 17),
5′-CACATCAGAGGAGACTGCCA-3′
(SEQ ID NO: 18),
5′-CCACATCAGAGGAGACTGCC-3′
(SEQ ID NO: 19),
5′-ATCAAAGGAGACTGCCGGTG-3′
(SEQ ID NO: 20),
5′-CATCAAAGGAGACTGCCGGT-3′
(SEQ ID NO: 21),
5′-ACATCAAAGGAGACTGCCGG-3′
(SEQ ID NO: 22),
5′-CACATCAAAGGAGACTGCCG-3′
(SEQ ID NO: 23), or
5′-CCACATCAAAGGAGACTGCC-3′
(SEQ ID NO: 24).

13. The method of the claim 10, wherein the nucleic acid sequence of the guide sequence includes at least one of:
5′-AUCAGAGGAGACUGCCAGUG-3′
(SEQ ID NO: 25),
5′-CAUCAGAGGAGACUGCCAGU-3′
(SEQ ID NO: 26),
5′-ACAUCAGAGGAGACUGCCAG-3′
(SEQ ID NO: 27),
5′-CACAUCAGAGGAGACUGCCA-3′
(SEQ ID NO: 28),
5′-CCACAUCAGAGGAGACUGCC-3′
(SEQ ID NO: 29),
5′-AUCAAAGGAGACUGCCGGUG-3′
(SEQ ID NO: 30),
5′-CAUCAAAGGAGACUGCCGGU-3′
(SEQ ID NO: 31),
5′-ACAUCAAAGGAGACUGCCGG-3′
(SEQ ID NO: 32),
5′-CACAUCAAAGGAGACUGCCG-3′
(SEQ ID NO: 33), or
5′-CCACAUCAAAGGAGACUGCC-3′
(SEQ ID NO: 34).

14. A method of restoring cone function or prolonging cone survival in a subject with an IRD-related cone or cone-rod dystrophy associated with a pathogenic point mutation in a mutant allele of an IRD-related gene in the retina or the retinal pigment epithelium (RPE), the method comprising:
base editing the pathogenic point mutation in the retinal cell or retinal pigment epithelium cell to correct the pathogenic mutation, generate a non-pathogenic point mutation, or modulate expression of an IRD-related gene and restore visual function of subject.

15. The method of claim 14, where the pathogenic mutation is a nonsense or missense mutation of RPE65 and the base editing increases expression of RPE65 in the retinal cell or retinal pigment epithelium cell by at least about 4%, 5%, 6%, 7 %, 8%, 9%, 10%, 20%, 30%, 40% or more.

16. The method of claim 14, wherein the base editing cause less than 3%, less than 2%, or less than 1% indel formation.

17. The method of claim 14, wherein the base editing comprises subretinal injecting at least one vector encoding a base editor and guide RNA that hybridizes to or is complementary to a target nucleic acid sequence of the mutant RPE65, which includes the point mutation.

18. The method of claim 17, wherein the pathogenic mutation comprises a C to T missense or nonsense mutation of the RPE65 gene and base editing by deamination of the A complementary to the T by the base editor and the guide RNA corrects the C to T mutation.

19. The method of claim 17, wherein the nucleic acid sequence of the target sequence includes at least one of:
5′-CTCACTGGCAGTCTCCTCTGATGTGGGCCA -3′
(SEQ ID NO: 1);
5′-CTCACCGGCAGTCTCCTCTGATGTGGGCCA -3′
(SEQ ID NO:2);
5′- TCACTGGCAGTCTCCTCTGATGTGGGCCAG-3′
(SEQ ID NO: 3);
5′- CACTGGCAGTCTCCTCTGATGTGGGCCAGG-3′
(SEQ ID NO: 4);
5′-ACTGGCAGTCTCCTCTGATGTGGGCCAGGG-3′
(SEQ ID NO: 5);
5′- GCTCACTGGCAGTCTCCTCTGATGTGGGCC-3′
(SEQ ID NO: 6);
5′- GGCTCACTGGCAGTCTCCTCTGATGTGGGC-3′
(SEQ ID NO: 7);
5′- TGGCTCACTGGCAGTCTCCTCTGATGTGGG-3′
(SEQ ID NO: 8);
5′- TCACCGGCAGTCTCCTTTGATGTGGGCCAG-3′
(SEQ ID NO: 9);
5′- CACCGGCAGTCTCCTTTGATGTGGGCCAGG-3′
(SEQ ID NO: 10);
5′-ACCGGCAGTCTCCTTTGATGTGGGCCAGGG-3′
(SEQ ID NO: 11);
5′- GCTCACCGGCAGTCTCCTTTGATGTGGGCC-3′
(SEQ ID NO: 12);
5′- GGCTCACCGGCAGTCTCCTTTGATGTGGGC-3′
(SEQ ID NO: 13); or
5′- TGGCTCACCGGCAGTCTCCTTTGATGTGGGC-3′
(SEQ ID NO: 14).

20. The method of claim 17, wherein the nucleic acid sequence of DNA encoding the guide sequence includes at least one of:
5′-ATCAGAGGAGACTGCCAGTG-3′
(SEQ ID NO: 15),
5′-CATCAGAGGAGACTGCCAGT-3′
(SEQ ID NO: 16),
5′-ACATCAGAGGAGACTGCCAG-3′
(SEQ ID NO: 17),
5′-CACATCAGAGGAGACTGCCA-3′
(SEQ ID NO: 18),
5′-CCACATCAGAGGAGACTGCC-3′
(SEQ ID NO: 19),
5′-ATCAAAGGAGACTGCCGGTG-3′
(SEQ ID NO: 20),
5′-CATCAAAGGAGACTGCCGGT-3′
(SEQ ID NO: 21),
5′-ACATCAAAGGAGACTGCCGG-3′
(SEQ ID NO: 22),
5′-CACATCAAAGGAGACTGCCG-3′
(SEQ ID NO: 23), or
5′-CCACATCAAAGGAGACTGCC-3′
(SEQ ID NO: 24).

21. The method of the claim 17, wherein the nucleic acid sequence of the guide sequence includes at least one of:
5′-AUCAGAGGAGACUGCCAGUG-3′
(SEQ ID NO: 25),
5′-CAUCAGAGGAGACUGCCAGU-3′
(SEQ ID NO: 26),
5′-ACAUCAGAGGAGACUGCCAG-3′
(SEQ ID NO: 27),
5′-CACAUCAGAGGAGACUGCCA-3′
(SEQ ID NO: 28),
5′-CCACAUCAGAGGAGACUGCC-3′
(SEQ ID NO: 29),
5′-AUCAAAGGAGACUGCCGGUG-3′
(SEQ ID NO: 30),
5′-CAUCAAAGGAGACUGCCGGU-3′
(SEQ ID NO: 31),
5′-ACAUCAAAGGAGACUGCCGG-3′
(SEQ ID NO: 32),
5′-CACAUCAAAGGAGACUGCCG-3′
(SEQ ID NO: 33), or
5′-CCACAUCAAAGGAGACUGCC-3′
(SEQ ID NO: 34).

22. The method of claim 17, wherein base editing the pathogenic mutated gene of a retinal cell or retinal pigment epithelium (RPE) cell can increase arrestin expression in the retina cells or retinal pigment epithelium cells of the subject being treated.

23. A complex comprising a fusion protein that includes a nucleic acid programmable DNA binding protein and an adenosine deaminase and a guide sequence comprising the nucleic sequence of at least one of:
5′-AUCAGAGGAGACUGCCAGUG-3′
(SEQ ID NO: 25),
5′-CAUCAGAGGAGACUGCCAGU-3′
(SEQ ID NO: 26),
5′-ACAUCAGAGGAGACUGCCAG-3′
(SEQ ID NO: 27),
5′-CACAUCAGAGGAGACUGCCA-3′
(SEQ ID NO: 28),
5′-CCACAUCAGAGGAGACUGCC-3′
(SEQ ID NO: 29),
5′-AUCAAAGGAGACUGCCGGUG-3′
(SEQ ID NO: 30),
5′-CAUCAAAGGAGACUGCCGGU-3′
(SEQ ID NO: 31),
5′-ACAUCAAAGGAGACUGCCGG-3′
(SEQ ID NO: 32),
5′-CACAUCAAAGGAGACUGCCG-3′
(SEQ ID NO: 33), or
5′-CCACAUCAAAGGAGACUGCC-3′
(SEQ ID NO: 34).

24. A guide sequence comprising the nucleic sequence of at least one of:
5′-AUCAGAGGAGACUGCCAGUG-3′
(SEQ ID NO: 25),
5′-CAUCAGAGGAGACUGCCAGU-3′
(SEQ ID NO: 26),
5′-ACAUCAGAGGAGACUGCCAG-3′
(SEQ ID NO: 27),
5′-CACAUCAGAGGAGACUGCCA-3′
(SEQ ID NO: 28),
5′-CCACAUCAGAGGAGACUGCC-3′
(SEQ ID NO: 29),
5′-AUCAAAGGAGACUGCCGGUG-3′
(SEQ ID NO: 30),
5′-CAUCAAAGGAGACUGCCGGU-3′
(SEQ ID NO: 31),
5′-ACAUCAAAGGAGACUGCCGG-3′
(SEQ ID NO: 32),
5′-CACAUCAAAGGAGACUGCCG-3′
(SEQ ID NO: 33), or
5′-CCACAUCAAAGGAGACUGCC-3′
(SEQ ID NO: 34).

25. A vector encoding a guide sequence of comprising the nucleic sequence of at least one of:
5′-AUCAGAGGAGACUGCCAGUG-3′
(SEQ ID NO: 25),
5′-CAUCAGAGGAGACUGCCAGU-3′
(SEQ ID NO: 26),
5′-ACAUCAGAGGAGACUGCCAG-3′
(SEQ ID NO: 27),
5′-CACAUCAGAGGAGACUGCCA-3′
(SEQ ID NO: 28),
5′-CCACAUCAGAGGAGACUGCC-3′
(SEQ ID NO: 29),
5′-AUCAAAGGAGACUGCCGGUG-3′
(SEQ ID NO: 30),
5′-CAUCAAAGGAGACUGCCGGU-3′
(SEQ ID NO: 31),
5′-ACAUCAAAGGAGACUGCCGG-3′
(SEQ ID NO: 32),
5′-CACAUCAAAGGAGACUGCCG-3′
(SEQ ID NO: 33), or
5′-CCACAUCAAAGGAGACUGCC-3′
(SEQ ID NO: 34).


SEQUENCE LISTING (52 sequences)

SEQ ID NO: 1 - DNA, 30 bp, Artificial Sequence
  Synthetic Construct
ctcactggcagtctcctctgatgtgggcca

SEQ ID NO: 2 - DNA, 30 bp, Artificial Sequence
  Synthetic Construct
ctcaccggcagtctcctctgatgtgggcca

SEQ ID NO: 3 - DNA, 30 bp, Artificial Sequence
  Synthetic Construct
tcactggcagtctcctctgatgtgggccag

SEQ ID NO: 4 - DNA, 30 bp, Artificial Sequence
  Synthetic Construct
cactggcagtctcctctgatgtgggccagg

SEQ ID NO: 5 - DNA, 30 bp, Artificial Sequence
  Synthetic Construct
actggcagtctcctctgatgtgggccaggg

SEQ ID NO: 6 - DNA, 30 bp, Artificial Sequence
  Synthetic Construct
gctcactggcagtctcctctgatgtgggcc

SEQ ID NO: 7 - DNA, 30 bp, Artificial Sequence
  Synthetic Construct
ggctcactggcagtctcctctgatgtgggc

SEQ ID NO: 8 - DNA, 30 bp, Artificial Sequence
  Synthetic Construct
tggctcactggcagtctcctctgatgtggg

SEQ ID NO: 9 - DNA, 30 bp, Artificial Sequence
  Synthetic Construct
tcaccggcagtctcctttgatgtgggccag

SEQ ID NO: 10 - DNA, 30 bp, Artificial Sequence
  Synthetic Construct
caccggcagtctcctttgatgtgggccagg

SEQ ID NO: 11 - DNA, 30 bp, Artificial Sequence
  Synthetic Construct
accggcagtctcctttgatgtgggccaggg

SEQ ID NO: 12 - DNA, 30 bp, Artificial Sequence
  Synthetic Construct
gctcaccggcagtctcctttgatgtgggcc

SEQ ID NO: 13 - DNA, 30 bp, Artificial Sequence
  Synthetic Construct
ggctcaccggcagtctcctttgatgtgggc

SEQ ID NO: 14 - DNA, 31 bp, Artificial Sequence
  Synthetic Construct
tggctcaccggcagtctcctttgatgtgggc

SEQ ID NO: 15 - DNA, 20 bp, Artificial Sequence
  Synthetic Construct
atcagaggagactgccagtg

SEQ ID NO: 16 - DNA, 20 bp, Artificial Sequence
  Synthetic Construct
catcagaggagactgccagt

SEQ ID NO: 17 - DNA, 20 bp, Artificial Sequence
  Synthetic Construct
acatcagaggagactgccag

SEQ ID NO: 18 - DNA, 20 bp, Artificial Sequence
  Synthetic Construct
cacatcagaggagactgcca

SEQ ID NO: 19 - DNA, 20 bp, Artificial Sequence
  Synthetic Construct
ccacatcagaggagactgcc

SEQ ID NO: 20 - DNA, 20 bp, Artificial Sequence
  Synthetic Construct
atcaaaggagactgccggtg

SEQ ID NO: 21 - DNA, 20 bp, Artificial Sequence
  Synthetic Construct
catcaaaggagactgccggt

SEQ ID NO: 22 - DNA, 20 bp, Artificial Sequence
  Synthetic Construct
acatcaaaggagactgccgg

SEQ ID NO: 23 - DNA, 20 bp, Artificial Sequence
  Synthetic Construct
cacatcaaaggagactgccg

SEQ ID NO: 24 - DNA, 20 bp, Artificial Sequence
  Synthetic Construct
ccacatcaaaggagactgcc

SEQ ID NO: 25 - RNA, 20 nt, Artificial Sequence
  Synthetic Construct
aucagaggagacugccagug

SEQ ID NO: 26 - RNA, 20 nt, Artificial Sequence
  Synthetic Construct
caucagaggagacugccagu

SEQ ID NO: 27 - RNA, 20 nt, Artificial Sequence
  Synthetic Construct
acaucagaggagacugccag

SEQ ID NO: 28 - RNA, 20 nt, Artificial Sequence
  Synthetic Construct
cacaucagaggagacugcca

SEQ ID NO: 29 - RNA, 20 nt, Artificial Sequence
  Synthetic Construct
ccacaucagaggagacugcc

SEQ ID NO: 30 - RNA, 20 nt, Artificial Sequence
  Synthetic Construct
aucaaaggagacugccggug

SEQ ID NO: 31 - RNA, 20 nt, Artificial Sequence
  Synthetic Construct
caucaaaggagacugccggu

SEQ ID NO: 32 - RNA, 20 nt, Artificial Sequence
  Synthetic Construct
acaucaaaggagacugccgg

SEQ ID NO: 33 - RNA, 20 nt, Artificial Sequence
  Synthetic Construct
cacaucaaaggagacugccg

SEQ ID NO: 34 - RNA, 20 nt, Artificial Sequence
  Synthetic Construct
ccacaucaaaggagacugcc

SEQ ID NO: 35 - DNA, 23 bp, Artificial Sequence
  Synthetic Construct
tgcttcgactccgtggaaatctc

SEQ ID NO: 36 - DNA, 22 bp, Artificial Sequence
  Synthetic Construct
gactttcctcttcttcttgggc

SEQ ID NO: 37 - DNA, 25 bp, Artificial Sequence
  Synthetic Construct
caccgacatcagaggagactgccag

SEQ ID NO: 38 - DNA, 25 bp, Artificial Sequence
  Synthetic Construct
aaacctggcagtctcctctgatgtc

SEQ ID NO: 39 - DNA, 15 bp, Artificial Sequence
  Synthetic Construct
ctcctctgatgtggg

SEQ ID NO: 40 - DNA, 15 bp, Artificial Sequence
  Synthetic Construct
ctcctccgatgtggg

SEQ ID NO: 41 - DNA, 15 bp, Artificial Sequence
  Synthetic Construct
ctcctccgatgtggg

SEQ ID NO: 42 - DNA, 15 bp, Artificial Sequence
  Synthetic Construct
ctcccctgatgtggg

SEQ ID NO: 43 - DNA, 16 bp, Artificial Sequence
  Synthetic Construct
ctcccccgatgtgggg

SEQ ID NO: 44 - DNA, 15 bp, Artificial Sequence
  Synthetic Construct
ctcctctgatgtggg

SEQ ID NO: 45 - DNA, 37 bp, Artificial Sequence
  Synthetic Construct
tcactggcagtctcctctgatgtgggccagggctctt

SEQ ID NO: 46 - DNA, 37 bp, Artificial Sequence
  Synthetic Construct
tcactggcagtcttctccgatgtgggccagggctctt

SEQ ID NO: 47 - DNA, 37 bp, Artificial Sequence; identical to SEQ ID NO: 45
  Synthetic Construct

SEQ ID NO: 48 - DNA, 37 bp, Artificial Sequence
  Synthetic Construct
tcactggcagtctcctctgaatgtgggccagggctct

SEQ ID NO: 49 - DNA, 35 bp, Artificial Sequence
  Synthetic Construct
tcactggcagtctcctcatgtgggccagggctctt

SEQ ID NO: 50 - DNA, 36 bp, Artificial Sequence
  Synthetic Construct
tcactggcagtctcctctatgtgggccagggctctt

SEQ ID NO: 51 - DNA, 37 bp, Artificial Sequence; identical to SEQ ID NO: 46
  Synthetic Construct

SEQ ID NO: 52 - DNA, 20 bp, Artificial Sequence
  Synthetic Construct
acatcagaggagactgccag